(19) United States
(12) Patent Application Publication
(10) Pub. No.: US 2002/0068345 A1
(43) Pub. Date: Jun. 6, 2002

(54) 98P7C3: homeodomain protein highly expressed in various cancers

(76) Inventors: Pia M. Challita-Eid, Encino, CA (US); Rene S. Hubert, Los Angeles, CA (US); Mary Faris, Los Angeles, CA (US); Daniel E.H. Afar, Brisbane, CA (US); Elana Levin, Los Angeles, CA (US); Steve Chappell Mitchell, Santa Monica, CA (US); Aya Jakobovits, Beverly Hills, CA (US)

Correspondence Address: GATES & COOPER LLP, HOWARD HUGHES CENTER, 6701 CENTER DRIVE WEST, SUITE 1050, LOS ANGELES, CA, 90045, US

(21) Appl. No.: 09/866,359

(22) Filed: May 24, 2001

Related U.S. Application Data
(60) Provisional application No. 60/207,138, filed on May 24, 2000.

(51) Int. Cl. C12P 21/02; C12N 9/00; C12N 5/06

(52) U.S. Cl. 435/183; 435/325; 435/320.1; 435/69.1

(57) ABSTRACT

A novel gene (designated 98P7C3) and its encoded protein are described. While 98P7C3 exhibits tissue-restricted expression in normal adult tissue, it is aberrantly expressed in multiple cancers including prostate, bladder, kidney, lung, breast, uterine, cervical, stomach, rectal and colon cancers. Consequently, 98P7C3 provides a diagnostic and/or therapeutic target for cancers, and the 98P7C3 gene or fragment thereof, or its encoded protein or a fragment thereof used to elicit an immune response.

This application claims the benefit of U.S. provisional application No. 60/207,138, filed May 24, 2000, the entire contents of which are incorporated herein by reference.

### FIELD OF THE INVENTION

The invention described herein relates to a novel gene and its encoded protein, termed 98P7C3, and to diagnostic and therapeutic methods and compositions useful in the management of various cancers that express 98P7C3.

### BACKGROUND OF THE INVENTION

Cancer is the second leading cause of human death next to coronary disease. Worldwide, millions of people die from cancer every year. In the United States alone, cancer causes the death of well over a half-million people annually, with some 1.4 million new cases diagnosed per year. While deaths from heart disease have been declining significantly, those resulting from cancer generally are on the rise. In the early part of the next century, cancer is predicted to become the leading cause of death.

Worldwide, several cancers stand out as the leading killers. In particular, carcinomas of the lung, prostate, breast, colon, pancreas, and ovary represent the primary causes of cancer death. These and virtually all other carcinomas share a common lethal feature. With very few exceptions, metastatic disease from a carcinoma is fatal. Moreover, even for those cancer patients who initially survive their primary cancers, common experience has shown that their lives are dramatically altered. Many cancer patients experience strong anxieties driven by the awareness of the potential for recurrence or treatment failure. Many cancer patients experience physical debilitations following treatment. Furthermore, many cancer patients experience a recurrence.

Worldwide, prostate cancer is the fourth most prevalent cancer in men. In North America and Northern Europe, it is by far the most common cancer in males and is the second leading cause of cancer death in men. In the United States alone, well over 40,000 men die annually of this disease—second only to lung cancer. Despite the magnitude of these Figures, there is still no effective treatment for metastatic prostate cancer. Surgical prostatectomy, radiation therapy, hormone ablation therapy, surgical castration and chemotherapy continue to be the main treatment modalities. Unfortunately, these treatments are ineffective for many and are often associated with undesirable consequences.

On the diagnostic front, the lack of a prostate tumor marker that can accurately detect early-stage, localized tumors remains a significant limitation in the diagnosis and management of this disease. Although the serum prostate specific antigen (PSA) assay has been a very useful tool, however its specificity and general utility is widely regarded as lacking in several important respects.

Progress in identifying additional specific markers for prostate cancer has been improved by the generation of prostate cancer xenografts that can recapitulate different stages of the disease in mice. The LAPC (Los Angeles Prostate Cancer) xenografts are prostate cancer xenografts that have survived passage in severe combined immune deficient (SCID) mice and have exhibited the capacity to mimic the transition from androgen dependence to androgen independence (Klein et al., 1997, Nat. Med. 3:402). More recently identified prostate cancer markers include PCTA-1 (Su et al., 1996, Proc. Natl. Acad. Sci. USA 93: 7252), prostate-specific membrane (PSM) antigen (Pinto et al., Clin Cancer Res Sep. 2, 1996 (9): 1445-51), STEAP (Hubert, et al., Proc Natl Acad Sci USA. Dec. 7, 1999; 96(25): 14523-8) and prostate stem cell antigen (PSCA) (Reiter et al., 1998, Proc. Natl. Acad. Sci. USA 95: 1735).

While previously identified markers such as PSA, PSM, PCTA and PSCA have facilitated efforts to diagnose and treat prostate cancer, there is need for the identification of additional markers and therapeutic targets for prostate and related cancers in order to further improve diagnosis and therapy.

### SUMMARY OF THE INVENTION

The present invention relates to a novel gene, designated 98P7C3, that is over-expressed in multiple cancers listed in Table I. Northern blot expression analysis of 98P7C3 gene expression in normal tissues shows a restricted expression pattern in adult tissues. The nucleotide (FIG. 2) and amino acid (FIG. 2 and FIG. 3) sequences of 98P7C3 are provided. The tissue-related profile of 98P7C3 in normal adult tissues, combined with the over-expression observed in prostate and other tumors, shows that 98P7C3 is aberrantly over-expressed in at least some cancers, and thus serves as a useful diagnostic and/or therapeutic target for cancers of the tissues such as those listed in Table I.

The invention provides polynucleotides corresponding or complementary to all or part of the 98P7C3 genes, mRNAs, and/or coding sequences, preferably in isolated form, including polynucleotides encoding 98P7C3-related proteins and fragments of 4, 5, 6, 7, 8, 9, 10, 11, 12, 13, 14, 15, 16, 17, 18, 19, 20, 21, 22, 23, 24, 25, or more than 25 contiguous amino acids; at least 30, 35, 40, 45, 50, 55, 60, 65, 70, 80, 85, 90, 95, 100 or more than 100 contiguous amino acids of a 98P7C3-related protein, as well as the peptides/proteins themselves; DNA, RNA, DNA/RNA hybrids, and related molecules, polynucleotides or oligonucleotides complementary or having at least a 90% homology to the 98P7C3 genes or mRNA sequences or parts thereof, and polynucleotides or oligonucleotides that hybridize to the 98P7C3 genes, mRNAs, or to 98P7C3-encoding polynucleotides. Also provided are means for isolating cDNAs and the genes encoding 98P7C3. Recombinant DNA molecules containing 98P7C3 polynucleotides, cells transformed or transduced with such molecules, and host-vector systems for the expression of 98P7C3 gene products are also provided. The invention further provides antibodies that bind to 98P7C3 proteins and polypeptide fragments thereof, including polyclonal and monoclonal antibodies, murine and other mammalian antibodies, chimeric antibodies, humanized and fully human antibodies, and antibodies labeled with a detectable marker.

The invention further provides methods for detecting the presence and status of 98P7C3 polynucleotides and proteins in various biological samples, as well as methods for identifying cells that express 98P7C3. A typical embodiment of this invention provides methods for monitoring 98P7C3 gene products in a tissue or hematology sample having or suspected of having some form of growth dysregulation such as cancer.

The invention further provides various immunogenic or therapeutic compositions and strategies for treating cancers that express 98P7C3 such as prostate cancers, including therapies aimed at inhibiting the transcription, translation, processing or function of 98P7C3 as well as cancer vaccines.

### BRIEF DESCRIPTION OF THE FIGURES

FIG. 1. 98P7C3 SSH sequence. The SSH experiment was performed with cDNA digested with DPN II. The 98P7C3 sequence contains 188 bp.

FIGS. 2A-B. The cDNA and amino acid sequence of 98P7C3.

FIG. 3. The amino acid sequence encoded by the open reading frame of the nucleic acid sequence set forth in FIG. 2.

FIG. 4. Sequence alignment of 98P7C3 with human PAX6.

FIG. 5. Sequence alignment of 98P7C3 with newt goosecoid homeobox protein.

FIGS. 6A-C. Northern blot analysis of 98P7C3 expression in various normal human tissues (using the 98P7C3 SSH fragment as a probe) and LAPC xenografts. Two multiple tissue northern blots (Clontech) with 2 μg of mRNA/lane, and LAPC xenograft northern blots with 10 μg of total RNA/lane were probed with the 98P7C3 SSH fragment. Size standards in kilobases (kb) are indicated on the side. For FIG. 6A lanes represent: 1) heart; 2) brain; 3) placenta; 4) lung; 5) liver; 6) skeletal muscle; 7) kidney; 8) pancreas. For FIG. 6B lanes represent: 1) spleen; 2) thymus; 3) prostate; 4) testis; 5) ovary; 6) small intestine; 7) colon; 8) leukocytes. For FIG. 6C lanes represent: 1) prostate; 2) LAPC-4AD; 3) LAPC-4AI; 4) LAPC-9AD; 5) LAPC-9 AI.

FIG. 7. Northern blot analysis of 98P7C3 expression in LAPC xenografts that were grown subcutaneously (sc) or intra-tibially (it) within the mouse bone. Northern blots with 10 μg of total RNA/lane were probed with the 98P7C3 SSH fragment. Size standards in kilobases (kb) are indicated on the side. Lanes represent: 1) LAPC-4 AD sc; 2) LAPC-4 AD sc; 3) LAPC-4 AD sc; 4) LAPC-4 AD it; 5) LAPC-4 AD it; 6) LAPC-4 AD it; 7) LAPC-4 AD²; 8) LAPC-9 AD sc; 9) LAPC-9 AD sc; 10) LAPC-9 AD it; 11) LAPC-9 AD it; 12) LAPC-9 AD it; 13) LAPC-3 AI sc; 14) LAPC-3 AI sc.

FIG. 8. Expression of 98P7C3 in cancer cell lines. RNA was extracted from the LAPC xenograft and a number of cancer cell lines. Northern blots with 10 μg of total RNA/lane were probed with the 98P7C3 SSH fragment. Size standards in kilobases (kb) are indicated on the side. Lanes represent: 1) LAPC-4 AD; 2) LAPC-4 AI; 3) LAPC-9 AD; 4) LAPC-9 AI; 5) LNCaP; 6) PC-3; 7) DU145; 8) TsuPr1; 9) LAPC-4 CL; 10) HPAC (pancreatic); 11) KCL 22 (leukemic); 12) HOS (bone); 13) CAMA-1 (breast).

FIG. 9. Expression of 98P7C3 in prostate cancer patient samples. RNA was extracted from the prostate tumors and their normal adjacent tissue derived from prostate cancer patients. Northern blots with 10 μg of total RNA/lane were probed with the 98P7C3 SSH fragment. Size standards in kilobases (kb) are indicated on the side. Lanes represent: 1) Patient 1, normal adjacent tissue; 2) Patient 1, Gleason 9 tumor; 3) Patient 2, normal adjacent tissue; 4) Patient 2, Gleason 7 tumor; 5) Patient 3, normal adjacent tissue ; 6) Patient 3, Gleason 7 tumor.

FIG. 10. Expression of 98P7C3 in prostate cancer patient samples. RNA was extracted from prostate cancer xenografts (LAPC-4AD, LAPC-4AI, LAPC-9AD and LAPC-9AI), normal prostate (N), prostate tumors (T) and their normal adjacent tissues (NAT) derived from prostate cancer patients. Northern blots with 10 μg of total RNA/lane were probed with the 98P7C3 sequences. Size standards in kilobases (kb) are indicated on the left side of the Figure. Results show expression of 98P7C3 in all patients and in all four xenografts. Patients (Pt.): Pt.1, Gleason score 6; Pt.2, Gleason score 7; Pt.3, Gleason score 7; Pt.4, Gleason score 8; Pt.5, Gleason score 9.

FIG. 11. Expression of 98P7C3 in kidney cancer patient samples. RNA was extracted from normal kidney (N), kidney tumors (T) and their normal adjacent tissues (NAT) derived from kidney cancer patients. Northern blots with 10 μg of total RNA/lane were probed with the 98P7C3 sequences. Size standards in kilobases (kb) are indicated on the left side of the Figure. Results show expression of 98P7C3 in all kidney specimens tested. Patients (Pt.): Pt.1, Papillary carcinoma, stage I; Pt.2, Invasive papillary carcinoma; Pt.3, Clear cell type grade 1/3, focally 2/3; Pt.4, Clear cell type, stage III; Pt.5, Clear cell type, stage III; Pt.6, Clear cell type, stage III.

FIG. 12. Expression of 98P7C3 in lung cancer patient samples. RNA was extracted from lung tumors (T) and their normal adjacent tissues (NAT) derived from lung cancer patients. Northern blots with 10 μg of total RNA/lane were probed with the 98P7C3 sequences. Size standards in kilobases (kb) are indicated on the left side of the Figure. Results show expression of 98P7C3 in all lung specimens tested. Patients (Pt.): Pt.1, Squamous carcinoma, stage IB; Pt.2, Squamous carcinoma, stage IIB; Pt3, Squamous carcinoma, stage IIIA; Pt.4, Squamous carcinoma, stage IB; Pt.5, Adenocarcinoma IV, stage IV.

FIG. 13. Expression of 98P7C3 in colon cancer patient samples. RNA was extracted from normal colon (N), colon tumors (T) and their normal adjacent tissues (NAT) derived from colon cancer patients. Northern blots with 10 μg of total RNA/lane were probed with the 98P7C3 sequences. Size standards in kilobases (kb) are indicated on the left side of the Figure. Results show expression of 98P7C3 in all colon specimens tested. Strong upregulation in tumor expression compared to normal adjacent tissues is seen in 3 out of 6 patients. Patients (Pt.): Pt.1, stage I; Pt.2, stage II; Pt.3, stage II; Pt.4, stage III; Pt.5, stage IV; Pt.6, stage IV;

FIG. 14. Expression of 98P7C3 in human patient cancer specimens and cancer cell lines. Expression of 98P7C3 was assayed in a panel of human cancers (T) and their respective matched normal tissues (N) on RNA dot blots. 98P7C3 expression was seen in kidney, breast, prostate, uterus, cervix, colon, lung, stomach and rectum cancers. The expression detected in some normal adjacent tissues (isolated from diseased tissues), but not in normal tissues (isolated from healthy donors), may indicate that these tissues are not fully normal and that 98P7C3 may be expressed in early stage tumors.

FIG. 15. Graphic depiction of amino acid scale profile of the 98P7C3 amino acid sequence. This profile, Hydropathicity (Kyte J., Doolittle R. F., 1982, J. Mol. Biol. 157:105-132), was used with others to identify antigenic regions of the 98P7C3 protein.

FIG. 16. Graphic depiction of amino acid scale profile of the 98P7C3 amino acid sequence. This profile, Average flexibility (Bhaskaran R., Ponnuswamy P. K., 1988, Int. J. Pept. Protein Res. 32:242-255), was used with others to identify antigenic regions of the 98P7C3 protein.

FIG. 17. Graphic depiction of amino acid scale profile of the 98P7C3 amino acid sequence. This profile, Beta-turn (Deleage, G., Roux B. 1987, Protein Engineering 1:289-294), was used with others to identify antigenic regions of the 98P7C3 protein.

FIG. 18. Graphic depiction of amino acid scale profile of the 98P7C3 amino acid sequence. This profile, Percentage Accessible residues (Janin J., 1979, Nature 277:491-492), was used with others to identify antigenic regions of the 98P7C3 protein.

FIG. 19. RT-PCR analysis of 98P7C3 expression in various pooled cancer tissues after 30 cycles. 98P7C3 expression was observed in each of the following tissues: xenograft pool (lane 1), prostate cancer pool (lane 2), bladder cancer pool (lane 3), kidney cancer pool (lane 4), colon cancer pool (lane 5), lung cancer pool (lane 6), ovarian cancer pool (lane 7), breast cancer pool (lane 8), metastasis pool (lane 9). Lane 10 is a water blank.

### DETAILED DESCRIPTION OF THE INVENTION

Outline of Sections

I.) Definitions

II.) Properties of 98P7C3.

III.) 98P7C3 Polynucleotides

III.A.) Uses of 98P7C3 Polynucleotides

III.A.1.) Monitoring of Genetic Abnormalities

III.A.2.) Antisense Embodiments

III.A.3.) Primers and Primer Pairs

III.A.4.) Isolation of 98P7C3-Encoding Nucleic Acid Molecules

II.A.5.) Recombinant Nucleic Acid Molecules and Host-Vector Systems

IV.) 98P7C3-related Proteins

IV.A.) Motif-bearing Protein Embodiments

IV.B.) Expression of 98P7C3-related Proteins

IV.C.) Modifications of 98P7C3-related Proteins

IV.D.) Uses of 98P7C3-related Proteins

V.) 98P7C3 Antibodies

VI.) 98P7C3 Transgenic Animals

VII.) Methods for the Detection of 98P7C3

VIII.) Methods for Monitoring the Status of 98P7C3-related Genes and Their Products

IX.) Identification of Molecules That Interact With 98P7C3

X.) Therapeutic Methods and Compositions

X.A.) 98P7C3 as a Target for Antibody-Based Therapy

X.B.) Anti-Cancer Vaccines

X.I.) Inhibition of 98P7C3 Protein Function

XI.A.) Inhibition of 98P7C3 With Intracellular Antibodies

XII.B.) Inhibition of 98P7C3 with Recombinant Proteins

XI.C.) Inhibition of 98P7C3 Transcription or Translation

XI.D.) General Considerations for Therapeutic Strategies

XII.) KITS

I.) Definitions:

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art, such as, for example, the widely utilized molecular cloning methodologies described in Sambrook et al., Molecular Cloning: A Laboratory Manual 2nd. edition (1989) Cold Spring Harbor Laboratory Press, Cold Spring Harbor, N.Y. As appropriate, procedures involving the use of commercially available kits and reagents are generally carried out in accordance with manufacturer defined protocols and/or parameters unless otherwise noted.

As used herein, the terms “advanced prostate cancer”, “locally advanced prostate cancer”, “advanced disease” and “locally advanced disease” mean prostate cancers that have extended through the prostate capsule, and are meant to include stage C disease under the American Urological Association (AUA) system, stage C1-C2 disease under the Whitmore-Jewett system, and stage T3-T4 and N+ disease under the TNM (tumor, node, metastasis) system. In general, surgery is not recommended for patients with locally advanced disease, and these patients have substantially less favorable outcomes compared to patients having clinically localized (organ-confmed) prostate cancer. Locally advanced disease is clinically identified by palpable evidence of induration beyond the lateral border of the prostate, or asymmetry or induration above the prostate base. Locally advanced prostate cancer is presently diagnosed pathologically following radical prostatectomy if the tumor invades or penetrates the prostatic capsule, extends into the surgical margin, or invades the seminal vesicles.

“Altering the native glycosylation pattern” is intended for purposes herein to mean deleting one or more carbohydrate moieties found in native sequence 98P7C3 (either by removing the underlying glycosylation site or by deleting the glycosylation by chemical and/or enzymatic means), and/or adding one or more glycosylation sites that are not present in the native sequence 98P7C3. In addition, the phrase includes qualitative changes in the glycosylation of the native proteins, involving a change in the nature and proportions of the various carbohydrate moieties present.

The term “analog” refers to a molecule which is structurally similar or shares similar or corresponding attributes with another molecule (e.g. a 98P7C3-related protein). For example an analog of the 98P7C3 protein can be specifically bound by an antibody or T cell that specifically binds to 98P7C3.

The term “antibody” is used in the broadest sense. Therefore an “antibody” can be naturally occurring or man-made such as monoclonal antibodies produced by conventional hybridoma technology. Anti-98P7C3 antibodies comprise monoclonal and polyclonal antibodies as well as fragments containing the antigen-binding domain and/or one or more complementarity determining regions of these antibodies.

As used herein, an “antibody fragment” is defined as at least a portion of the variable region of the immunoglobulin molecule that binds to its target, i.e., the antigen-binding region. In one embodiment it specifically covers single anti-98P7C3 antibodies and clones thereof (including agonist, antagonist and neutralizing antibodies) and anti-98P7C3 antibody compositions with polyepitopic specificity.

The term “codon optimized sequences” refers to nucleotide sequences that have been optimized for a particular host species by replacing any codons having a usage frequency of less than about 20%. Nucleotide sequences that have been optimized for expression in a given host species by elimination of spurious polyadenylation sequences, elimination of exon/intron splicing signals, elimination of transposon-like repeats and/or optimization of GC content in addition to codon optimization are referred to herein as an “expression enhanced sequences.”

The term “cytotoxic agent” as used herein refers to a substance that inhibits or prevents the function of cells and/or causes destruction of cells. The term is intended to include radioactive isotopes chemotherapeutic agents, and toxins such as small molecule toxins or enzymatically active toxins of bacterial, fungal, plant or animal origin, including fragments and/or variants thereof. Examples of cytotoxic agents include, but are not limited to maytansinoids, ytrium, bismuth ricin, ricin A-chain, doxorubicin, daunorubicin, taxol, ethidium bromide, mitomycin, etoposide, tenoposide, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, retstrictocin, phenomycin, enomycin, curicin, crotin, calicheamicin, sapaonaria officinalis inhibitor, and glucocorticoid and other chemotherapeutic agents, as well as radioisotopes such as At²¹¹, I¹³¹, I¹²⁵, Y⁹⁰, Re¹⁸⁶, Re¹⁸⁸, Sm¹⁵³, Bi²¹², P³² and radioactive isotopes of Lu. Antibodies may also be conjugated to an anti-cancer pro-drug activating enzyme capable of converting the pro-drug to its active form.

The term “homolog” refers to a molecule which exhibits homology to another molecule, by for example, having sequences of chemical residues that are the same or similar at corresponding positions.

As used herein, the terms “hybridize”, “hybridizing”, “hybridizes” and the like, used in the context of polynucleotides, are meant to refer to conventional hybridization conditions, preferably such as hybridization in 50% formamide/6×SSC/0.1% SDS/100 μg/ml ssDNA, in which temperatures for hybridization are above 37 degrees C. and temperatures for washing in 0.1×SSC/0.1% SDS are above 55 degrees C.

As used herein, a polynucleotide is said to be “isolated” when it is substantially separated from contaminant polynucleotides that correspond or are complementary to genes other than the 98P7C3 gene or that encode polypeptides other than 98P7C3 gene product or fragments thereof A skilled artisan can readily employ nucleic acid isolation procedures to obtain an isolated 98P7C3 polynucleotide.

As used herein, a protein is said to be “isolated” when physical, mechanical or chemical methods are employed to remove the 98P7C3 protein from cellular constituents that are normally associated with the protein. A skilled artisan can readily employ standard purification methods to obtain an isolated 98P7C3 protein. Alternatively, an isolated protein can be prepared by chemical means.

The term “mammal” as used herein refers to any organism classified as a mammal, including mice, rats, rabbits, dogs, cats, cows, horses and humans. In one embodiment of the invention, the mammal is a mouse. In another embodiment of the invention, the mammal is a human.

As used herein, the terms “metastatic prostate cancer” and “metastatic disease” mean prostate cancers that have spread to regional lymph nodes or to distant sites, and are meant to include stage D disease under the AUA system and stage T×N×M+ under the TNM system. As is the case with locally advanced prostate cancer, surgery is generally not indicated for patients with metastatic disease, and hormonal (androgen ablation) therapy is a preferred treatment modality. Patients with metastatic prostate cancer eventually develop an androgen-refractory state within 12 to 18 months of treatment initiation. Approximately half of these androgen-refractory patients die within 6 months after developing that status. The most common site for prostate cancer metastasis is bone. Prostate cancer bone metastases are often osteoblastic rather than osteolytic (i.e., resulting in net bone formation). Bone metastases are found most frequently in the spine, followed by the femur, pelvis, rib cage, skull and humerus. Other common sites for metastasis include lymph nodes, lung, liver and brain. Metastatic prostate cancer is typically diagnosed by open or laparoscopic pelvic lymphadenectomy, whole body radionuclide scans, skeletal radiography, and/or bone lesion biopsy.

The term “monoclonal antibody” as used herein refers to an antibody obtained from a population of substantially homogeneous antibodies, i.e., the antibodies comprising the population are identical except for possible naturally occurring mutations that are present in minor amounts.

As used herein “motif” as in biological motif of an 98P7C3-related protein, refers to any pattern of amino acids forming part of the primary sequence of a protein, that is associated with a particular function (e.g. protein-protein interaction, protein-DNA interaction, etc) or modification (e.g. that is phosphorylated, glycosylated or amidated), or localization (e.g. secretory sequence, nuclear localization sequence, etc.) or a sequence that is correlated with being immunogenic, either humorally or cellularly. A motif can be either contiguous or capable of being aligned to certain positions that are generally correlated with a certain function or property.

As used herein, the term “polynucleotide” means a polymeric form of nucleotides of at least 10 bases or base pairs in length, either ribonucleotides or deoxynucleotides or a modified form of either type of nucleotide, and is meant to include single and double stranded forms of DNA and/or RNA. In the art, this term if often used interchangeably with “oligonucleotide”. A polynucleotide can comprise a nucleotide sequence disclosed herein wherein thymidine (T) (as shown for example in SEQ ID NO: 1) can also be uracil (U); this definition pertains to the differences between the chemical structures of DNA and RNA, in particular the observation that one of the four major bases in RNA is uracil (U) instead of thymidine (T).

As used herein, the term “polypeptide” means a polymer of at least about 4, 5, 6, 7, or 8 amino acids. Throughout the specification, standard three letter or single letter designations for amino acids are used. In the art, this term is often used interchangeably with “peptide” or “protein”.

As used herein, a “recornbinant” DNA or RNA molecule is a DNA or RNA molecule that has been subjected to molecular manipulation in vitro.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured nucleic acid sequences to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, are identified by, but not limited to, those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1 % bovine serum albumin/0.1 % Ficoll/0.1 % polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium. citrate) and 50% formamide at 55° C., followed by a high-stringency wash consisting of 0.1×SSC containing EDTA at 55° C. “Moderately stringent conditions” are described by, but not limited to, those in Sambrook et al., Molecular Cloning: A Laboratory Manual, New York: Cold Spring Harbor Press, 1989, and include the use of washing solution and hybridization conditions (e.g., temperature, ionic strength and % SDS) less stringent than those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/mL denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

A “kansgenic animal” (e.g., a mouse or rat) is an animal having cells that contain a transgene, which transgene was introduced into the animal or an ancestor of the animal at a prenatal, e.g., an embryonic stage. A “transgene” is a DNA that is integrated into the genome of a cell from which a transgenic animal develops.

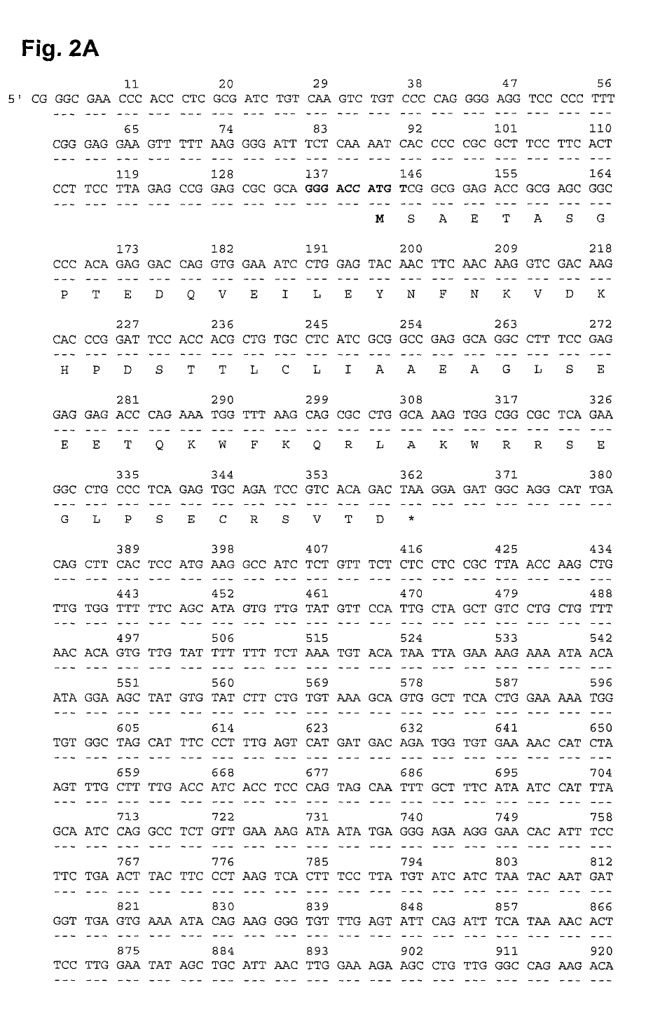

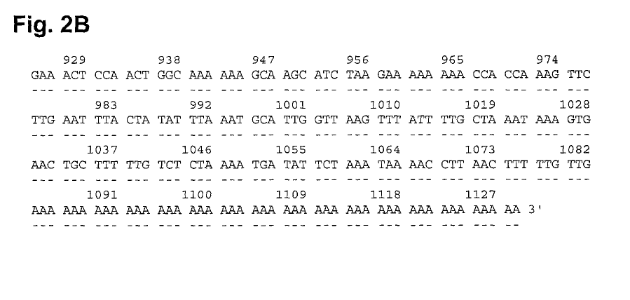

The term “variant” refers to a molecule that exhibits a variation from a described type or norm, such as a protein that has one or more different amino acid residues in the corresponding position(s) of a specifically described protein (e.g. the 98P7C3 protein shown in FIG. 2). An analog is an example of a variant protein.

As used herein, the 98P7C3-related gene and 98P7C3-related protein includes the 98P7C3 genes and proteins specifically described herein, as well as structurally and/or functionally similar variants or analog of the foregoing. 98P7C3 peptide analogs generally share at least about 50%, 60%, 70%, 80%, 90% or more amino acid homology (using BLAST criteria). 98P7C3 nucleotide analogs preferably share 50%, 60%, 70%, 80%, 90% or more nucleic acid homology (using BLAST criteria). In some embodiments, however, lower homology is preferred so as to select preferred residues in view of species-specific codon preferences and/or optimal peptide epitopes tailored to a particular target population, as is appreciated by those skilled in the art.

The 98P7C3-related proteins of the invention include those specifically identified herein, as well as allelic variants, conservative substitution variants, analogs and homologs that can be isolated/generated and characterized without undue experimentation following the methods outlined herein or readily available in the art. Fusion proteins that combine parts of different 98P7C3 proteins or fragments thereof, as well as fusion proteins of a 98P7C3 protein and a heterologous polypeptide are also included. Such 98P7C3 proteins are collectively referred to as the 98P7C3-related proteins, the proteins of the invention, or 98P7C3. As used herein, the term “98P7C3-related protein” refers to a polypeptide fragment or an 98P7C3 protein sequence of 4, 5, 6, 7, 8, 9, 10, 11, 12, 13, 14, 15, 16, 17, 18, 19, 20, 21,22,23, 24, 25, or more than 25 amino acids; or, at least 30, 35, 40, 45, 50, 55, 60, 65, 70, 80, 85, 90, 95, 100 or more than 100 amino acids.

II.) Properties of 98P7C3

As disclosed herein, 98P7C3 exhibits specific properties that are analogous to those found in a family of molecules whose polynucleotides, polypeptides, reactive cytotoxic T cells (CTL), reactive helper T cells (HTL) and anti-polypeptide antibodies are used in well known diagnostic assays that examine conditions associated with dysregulated cell growth such as cancer, in particular prostate cancer (see, e.g., both its highly specific pattern of tissue expression as well as its overexpression in prostate cancers as described for example in Example 4). The best-known member of this class is PSA, the archetypal marker that has been used by medical practitioners for years to identify and monitor the presence of prostate cancer (see, e.g., Merrill et al., J. Urol. 163(2): 503-5120 (2000); Polascik et al., J. Urol. Aug; 162(2):293-306 (1999) and Fortier et al., J. Nat. Cancer Inst. 91(19): 1635-1640(1999)). A variety of other diagnostic markers are also used in this context including p53 and K-ras (see, e.g., Tulchinsky et al., Int J Mol Med July 1999 4(1):99-102 and Minimoto et al., Cancer Detect Prev 2000;24(1):1-12). Therefore, this disclosure of the 98P7C3 polynucleotides and polypeptides (as well as the 98P7C3 polynucleotide probes and anti-98P7C3 antibodies used to identify the presence of these molecules) and their properties allows skilled artisans to utilize these molecules in methods that are analogous to those used, for example, in a variety of diagnostic assays directed to examining conditions associated with cancer.

Typical embodiments of diagnostic methods which utilize the 98P7C3 polynucleotides, polypeptides, reactive T cells and antibodies are analogous to those methods from well-established diagnostic assays which employ, e.g., PSA polynucleotides, polypeptides, reactive T cells and antibodies. For example, just as PSA polynucleotides are used as probes (for example in Northern analysis, see, e.g., Sharief et al., Biochem. Mol. Biol. Int. 33(3):567-74(1994)) and primers (for example in PCR analysis, see, e.g., Okegawa et al., J. Urol. 163(4): 1189-1190 (2000)) to observe the presence and/or the level of PSA mRNAs in methods of monitoring PSA overexpression or the metastasis of prostate cancers, the 98P7C3 polynucleotides described herein can be utilized in the same way to detect 98P7C3 overexpression or the metastasis of prostate and other cancers expressing this gene. Alternatively, just as PSA polypeptides are used to generate antibodies specific for PSA which can then be used to observe the presence and/or the level of PSA proteins in methods to monitor PSA protein overexpression (see, e.g., Stephan et al., Urology 55(4):560-3 (2000)) or the metastasis of prostate cells (see, e.g., Alanen et al., Pathol. Res. Pract. 192(3):233-7 (1996)), the 98P7C3 polypeptides described herein can be utilized to generate antibodies for use in detecting 98P7C3 overexpression or the metastasis of prostate cells and cells of other cancers expressing this gene.

Specifically, because metastases involves the movement of cancer cells from an organ of origin (such as the lung or prostate gland etc.) to a different area of the body (such as a lymph node), assays which examine a biological sample for the presence of cells expressing 98P7C3 polynucleotides and/or polypeptides can be used to provide evidence of metastasis. For example, when a biological sample from tissue that does not normally contain 98P7C3-expressing cells (lymph node) is found to contain 98P7C3-expressing cells such as the 98P7C3 expression seen in LAPC4 and LAPC9, xenografts isolated from lymph node and bone metastasis, respectively, this finding is indicative of metastasis.

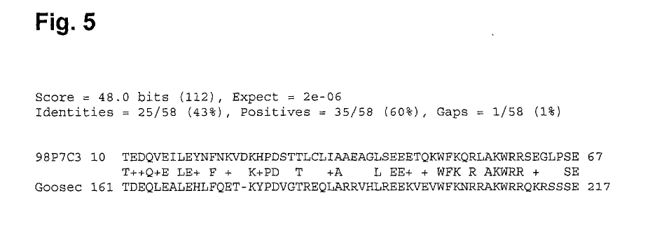

Alternatively 98P7C3 polynucleotides and/or polypeptides can be used to provide evidence of cancer, for example, when cells in a biological sample that do not normally express 98P7C3 or express 98P7C3 at a different level are found to express 98P7C3 or have an increased expression of 98P7C3 (see, e.g., the 98P7C3 expression in kidney, lung and colon cancer cells and in patient samples etc. shown in FIGS. 5-9). In such assays, artisans may further wish to generate supplementary evidence of metastasis by testing the biological sample for the presence of a second tissue restricted marker (in addition to 98P7C3) such as PSA, PSCA etc. (see, e.g., Alanen et al., Pathol. Res. Pract. 192(3): 233-237 (1996)).

Just as PSA polynucleotide fragments and polynucleotide variants are employed by skilled artisans for use in methods of monitoring PSA, 98P7C3 polynucleotide fragments and polynucleotide variants are used in an analogous manner. In particular, typical PSA polynucleotides used in methods of monitoring PSA are probes or primers which consist of fragments of the PSA cDNA sequence. Illustrating this, primers used to PCR amplify a PSA polynucleotide must include less than the whole PSA sequence to function in the polymerase chain reaction. In the context of such PCR reactions, skilled artisans generally create a variety of different polynucleotide fragments that can be used as primers in order to amplify different portions of a polynucleotide of interest or to optimize amplification reactions (see, e.g., Caetano-Anolles, G. Biotechniques 25(3): 472-476, 478-480 (1998); Robertson et al., Methods Mol. Biol. 98:121-154 (1998)). An additional illustration of the use of such fragments is provided in Example 4, where a 98P7C3 polynucleotide fragment is used as a probe to show the expression of 98P7C3 RNAs in cancer cells. In addition, variant polynucleotide sequences are typically used as primers and probes for the corresponding mRNAs in PCR and Northern analyses (see, e.g., Sawai et al., Fetal Diagn. Ther. November-December 1996 11(6):407-13 and Current Protocols In Molecular Biology, Volume 2, Unit 2, Frederick M. Ausubel et al. eds., 1995)). Polynucleotide fragments and variants are useful in this context where they are capable of binding to a target polynucleotide sequence (e.g. the 98P7C3 polynucleotide shown in SEQ ID NO: 1) under conditions of high stringency.

Furthermore, PSA polypeptides which contain an epitope that can be recognized by an antibody or T cell that specifically binds to that epitope are used in methods of monitoring PSA. 98P7C3 polypeptide fragments and polypeptide analogs or variants can also be used in an analogous manner. This practice of using polypeptide fragments or polypeptide variants to generate antibodies (such as anti-PSA antibodies or T cells) is typical in the art with a wide variety of systems such as fusion proteins being used by practitioners (see, e.g., Current Protocols In Molecular Biology, Volume 2, Unit 16, Frederick M. Ausubel et al. eds., 1995). In this context, each epitope(s) functions to provide the architecture with which an antibody or T cell is reactive. Typically, skilled artisans create a variety of different polypeptide fragments that can be used in order to generate immune responses specific for different portions of a polypeptide of interest (see, e.g., U.S. Pat. No. 5,840,501 and U.S. Pat. No. 5,939,533). For example it may be preferable to utilize a polypeptide comprising one of the 98P7C3 biological motifs discussed herein or available in the art. Polypeptide fragments, variants or analogs are typically useful in this context as long as they comprise an epitope capable of generating an antibody or T cell specific for a target polypeptide sequence (e.g. the 98P7C3 polypeptide shown in SEQ ID NO: 2).

As shown herein, the 98P7C3 polynucleotides and polypeptides (as well as the 98P7C3 polynucleotide probes and anti-98P7C3 antibodies or T cells used to identify the presence of these molecules) exhibit specific properties that make them useful in diagnosing cancers of the prostate. Diagnostic assays that measure the presence of 98P7C3 gene products, in order to evaluate the presence or onset of a disease condition described herein, such as prostate cancer, are used to identify patients for preventive measures or further monitoring, as has been done so successfully with PSA. Moreover, these materials satisfy a need in the art for molecules having similar or complementary characteristics to PSA in situations where, for example, a definite diagnosis of metastasis of prostatic origin cannot be made on the basis of a test for PSA alone (see, e.g., Alanen et al., Pathol. Res. Pract. 192(3): 233-237 (1996)), and consequently, materials such as 98P7C3 polynucleotides and polypeptides (as well as the 98P7C3 polynucleotide probes and anti-98P7C3 antibodies used to identify the presence of these molecules) must be employed to confirm metastases of prostatic origin.

Finally, in addition to their use in diagnostic assays, the 98P7C3 polynucleotides disclosed herein have a number of other specific utilities such as their use in the identification of oncogenetic associated chromosomal abnormalities in the chromosomal region to which the 98P7C3 gene maps (see Example 3 below). Moreover, in addition to their use in diagnostic assays, the 98P7C3-related proteins and polynucleotides disclosed herein have other utilities such as their use in the forensic analysis of tissues of unknown origin (see, e.g., Takahama K Forensic Sci Int June 1996 28;80(1-2): 63-9).

Additionally, 98P7C3-related proteins or polynucleotides of the invention can be used to treat a pathologic condition characterized by the over-expression of 98P7C3. For example, the amino acid or nucleic acid sequence of FIG. 2, or fragments thereof, can be used to generate an immune response to the 98P7C3 antigen. Antibodies or other molecules that react with 98P7C3 can be used to modulate the function of this molecule, and thereby provide a therapeutic benefit.

III.) 98P7C3 Polynucleotides

One aspect of the invention provides polynucleotides corresponding or complementary to all or part of an 98P7C3 gene, mRNA, and/or coding sequence, preferably in isolated form, including polynucleotides encoding an 98P7C3-related protein and fragments thereof, DNA, RNA, DNA/RNA hybrid, and related molecules, polynucleotides or oligonucleotides complementary to an 98P7C3 gene or mRNA sequence or a part thereof, and polynucleotides or oligonucleotides that hybridize to an 98P7C3 gene, mRNA, or to an 98P7C3 encoding polynucleotide (collectively, “98P7C3 polynucleotides”). In all instances when referred to in this section, T can also be U in FIG. 2.

Embodiments of a 98P7C3 polynucleotide include: a 98P7C3 polynucleotide having the sequence shown in FIG. 2, the nucleotide sequence of 98P7C3 as shown in FIG. 2, wherein T is U; at least 10 contiguous nucleotides of a polynucleotide having the sequence as shown in FIG. 2; or, at least 10 contiguous nucleotides of a polynucleotide having the sequence as shown in FIG. 2 where T is U. Embodiments of 98P7C3 nucleotides comprise, without limitation:

(a) a polynucleotide consisting of the sequence as shown in SEQ ID NO: 1, wherein T can also be U;

(b) a polynucleotide consisting of the sequence as shown in SEQ ID NO: 1, from nucleotide residue number 141 through nucleotide residue number 362, wherein T can also be U;

(c) a polynucleotide that encodes a 98P7C3-related protein whose sequence is encoded by the cDNAs contained in the plasmid designated p98P7C3-EBB10 deposited with American Type Culture Collection as Accession No. PTA- 1261;

(d) a polynucleotide that encodes an 98P7C3-related protein that is at least 90% homologous to the entire amino acid sequence shown in SEQ ID NO: 2;

(e) a polynucleotide that encodes an 98P7C3-related protein that is at least 90% identical to the entire amino acid sequence shown in SEQ ID NO: 2;

(f) a polynucleotide that encodes at least one peptide set forth in Tables V-XVIII;

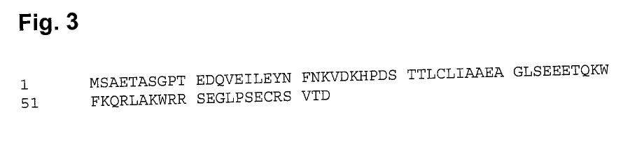

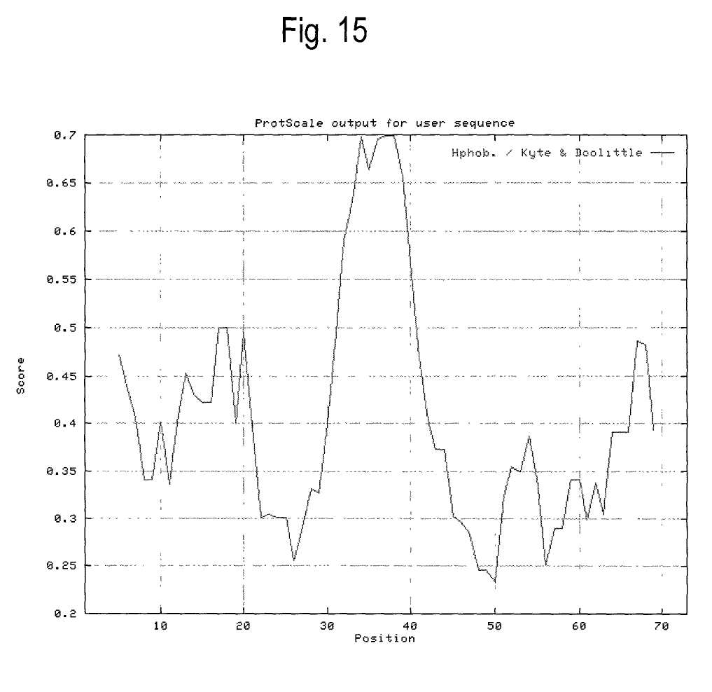

(g) a polynucleotide that encodes a peptide region of at least 5 amino acids of FIG. 3 in any whole number increment up to 73 that includes an amino acid position having a value less than 0.5 in the Hydropathicity profile of FIG. 15;

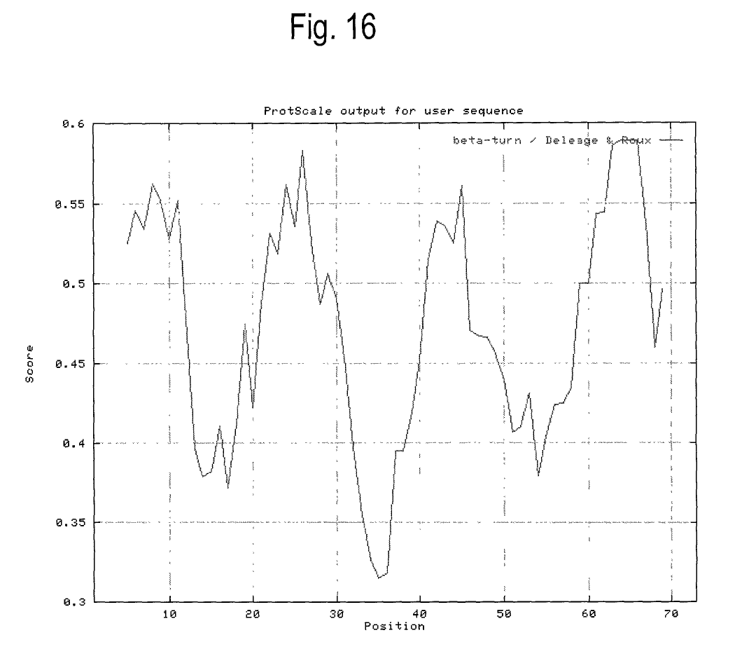

(h) a polynucleotide that encodes a peptide region of at least 5 amino acids of FIG. 3 in any whole number increment up to 73 that includes an amino acid position having a value greater than 0.5 in the Average Flexibility profile on FIG. 16;

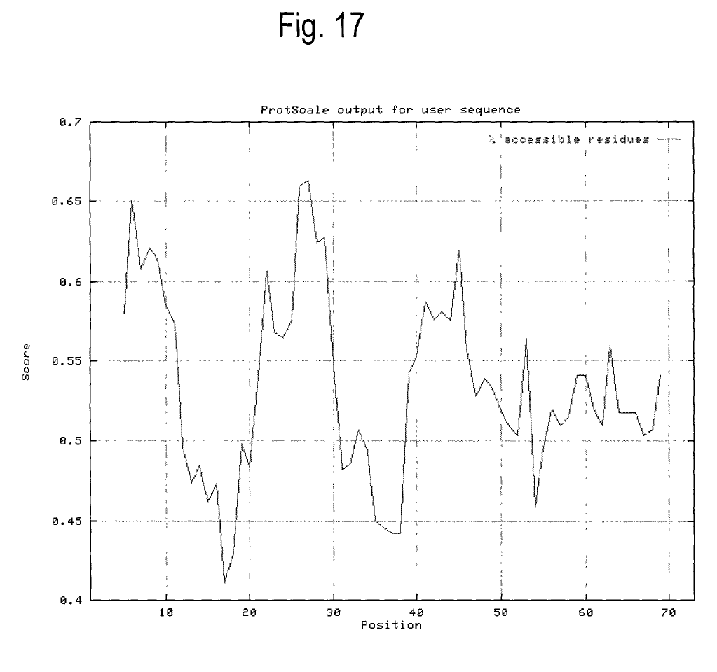

(i) a polynucleotide that encodes a peptide region of at least 5 amino acids of FIG. 3 in any whole number increment up to 73 that includes an amino acid position having a value greater than 0.5 in the Beta-turn profile of FIG. 17;

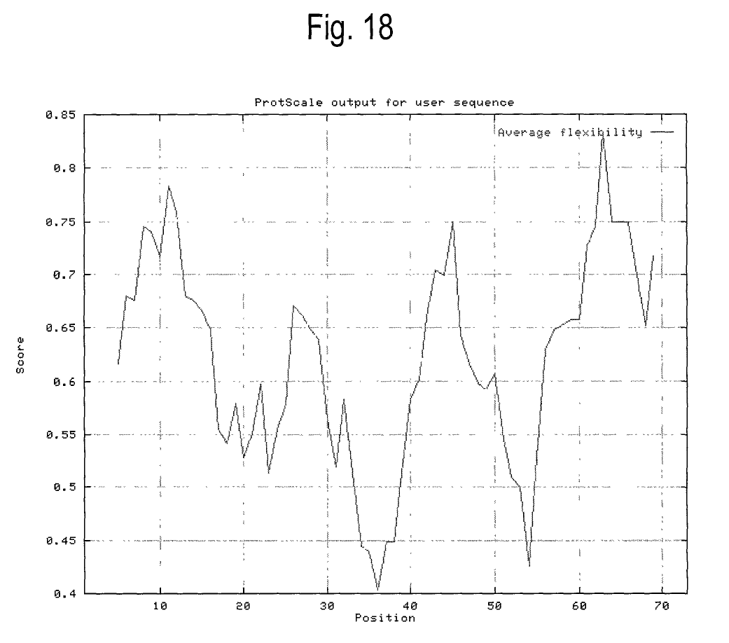

(j) a polynucleotide that encodes a peptide region of at least 5 amino acids of FIG. 3 in any whole number increment up to 73 that includes an amino acid position having a value greater than 0.5 in the Percentage Accessible Residues profile of FIG. 18;

(k) a polynucleotide that is fully complementary to a polynucleotide of any one of (a)-(j); and,

(l) a polynucleotide that selectively hybridizes under stringent conditions to a polynucleotide of (a)-(k).

(g) a polynucleotide that encodes a peptide region of at least 5 amino acids of FIG. 3 in any whole number increment up to 73 that includes an amino acid position having a value less than 0.5 on FIG. 15;

Moreover, a peptide that is encoded by any of the foregoing is also within the scope of the invention. As used herein, a range is understood to specifically disclose all whole unit positions thereof.

An alternative embodiment comprises a polynucleotide or protein/peptide of the invention together with a pharmaceutical excipient and/or in a human unit dose form.

Typical embodiments of the invention disclosed herein include 98P7C3 polynucleotides that encode specific portions of the 98P7C3 mRNA sequence (and those which are complementary to such sequences) such as those that encode the protein and fragments thereof, for example of 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, or 73 contiguous amino acids.

For example, representative embodiments of the invention disclosed herein include: polynucleotides and their encoded peptides themselves encoding about amino acid 1 to about amino acid 10 of the 98P7C3 protein shown in FIG. 2 or FIG. 3, polynucleotides encoding about amino acid 10 to about amino acid 20 of the 98P7C3 protein shown in FIG. 2 or FIG. 3, polynucleotides encoding about amino acid 20 to about amino acid 30 of the 98P7C3 protein shown in FIG. 2 or FIG. 3, polynucleotides encoding about amino acid 30 to about amino acid 40 of the 98P7C3 protein shown in FIG. 2 or FIG. 3, polynucleotides encoding about amino acid 40 to about amino acid 50 of the 98P7C3 protein shown in FIG. 2 or FIG. 3, polynucleotides encoding about amino acid 50 to about amino acid 60 of the 98P7C3 protein shown in FIG. 2 or FIG. 3, polynucleotides encoding about amino acid 60 to about amino acid 70 of the 98P7C3 protein shown in FIG. 2 or FIG. 3, in increments of about 10 amino acids, ending at the carboxyl terminal amino acid set forth in FIG. 2 or FIG. 3. Accordingly polynucleotides encoding portions of the amino acid sequence (of about 10 amino acids), of amino acids 70 through the carboxyl terminal amino acid of the 98P7C3 protein are embodiments of the invention. Wherein it is understood that each particular amino acid position discloses that specific position plus or minus five contiguous amino acid residues.

Polynucleotides encoding relatively long portions of the 98P7C3 protein are also within the scope of the invention. For example, polynucleotides encoding from about amino acid 1 (or 20 or 30 or 40 etc.) to about amino acid 20, (or 30, or 40 or 50 etc.) of the 98P7C3 protein shown in FIG. 2 can be generated by a variety of techniques well known in the art. These polynucleotide fragments can include any portion of the 98P7C3 sequence as shown in FIG. 2.

Additional illustrative embodiments of the invention disclosed herein include 98P7C3 polynucleotide fragments encoding one or more of the biological motifs contained within the 98P7C3 protein sequence, including one or more of the motif-bearing subsequences of the 98P7C3 protein set forth in Tables V-XIX. In another embodiment, typical polynucleotide fragments of the invention encode one or more of the regions of 98P7C3 that exhibit homology to a known molecule. In another embodiment of the invention, typical polynucleotide fragments can encode one or more of the 98P7C3 N-glycosylation sites, cAMP and cGMP-dependent protein kinase phosphorylation sites, casein kinase II phosphorylation sites or N-myristoylation site and amidation sites.

III.A.) Uses of 98P7C3 Polynucleotides

III.A.1.) Monitoring of Genetic Abnormalities

The polynucleotides of the preceding paragraphs have a number of different specific uses. The human 98P7C3 gene maps to the chromosomal location set forth in Example 3. For example, because the 98P7C3 gene maps to this chromosome, polynucleotides that encode different regions of the 98P7C3 protein are used to characterize cytogenetic abnormalities of this chromosomal locale, such as abnormalities that are identified as being associated with various cancers. In certain genes, a variety of chromosomal abnormalities including rearrangements have been identified as frequent cytogenetic abnormalities in a number of different cancers (see e.g. Krajinovic et al., Mutat. Res. 382(3-4): 81-83 (1998); Johansson et al., Blood 86(10): 3905-3914 (1995) and Finger et al., P.N.A.S. 85(23): 9158-9162 (1988)). Thus, polynucleotides encoding specific regions of the 98P7C3 protein provide new tools that can be used to delineate, with greater precision than previously possible, cytogenetic abnormalities in the chromosomal region that encodes 98P7C3 that may contribute to the malignant phenotype. In this context, these polynucleotides satisfy a need in the art for expanding the sensitivity of chromosomal screening in order to identify more subtle and less common chromosomal abnormalities (see e.g. Evans et al., Am. J. Obstet. Gynecol 171(4): 1055-1057 (1994)).

Furthermore, as 98P7C3 was shown to be highly expressed in prostate and other cancers, 98P7C3 polynucleotides are used in methods assessing the status of 98P7C3 gene products in normal versus cancerous tissues. Typically, polynucleotides that encode specific regions of the 98P7C3 protein are used to assess the presence of perturbations (such as deletions, insertions, point mutations, or alterations resulting in a loss of an antigen etc.) in specific regions of the 98P7C3 gene, such as such regions containing one or more motifs. Exemplary assays include both RT-PCR assays as well as single-strand conformation polymorphism (SSCP) analysis (see, e.g., Marrogi et al., J. Cutan. Pathol. 26(8): 369-378 (1999), both of which utilize polynucleotides encoding specific regions of a protein to examine these regions within the protein.

III.A.2.) Antisense Embodiments

Other specifically contemplated nucleic acid related embodiments of the invention disclosed herein are genomic DNA, cDNAs, ribozymes, and antisense molecules, as well as nucleic acid molecules based on an alternative backbone, or including alternative bases, whether derived from natural sources or synthesized, and include molecules capable of inhibiting the RNA or protein expression of 98P7C3. For example, antisense molecules can be RNAs or other molecules, including peptide nucleic acids (PNAs) or non-nucleic acid molecules such as phosphorothioate derivatives, that specifically bind DNA or RNA in a base pair-dependent manner. A skilled artisan can readily obtain these classes of nucleic acid molecules using the 98P7C3 polynucleotides and polynucleotide sequences disclosed herein.

Antisense technology entails the administration of exogenous oligonucleotides that bind to a target polynucleotide located within the cells. The term “antisense” refers to the fact that such oligonucleotides are complementary to their intracellular targets, e.g., 98P7C3. See for example, Jack Cohen, Oligodeoxynucleotides, Antisense Inhibitors of Gene Expression, CRC Press, 1989; and Synthesis 1:1-5 (1988). The 98P7C3 antisense oligonucleotides of the present invention include derivatives such as S-oligonucleotides (phosphorothioate derivatives or S-oligos, see, Jack Cohen, supra), which exhibit enhanced cancer cell growth inhibitory action. S-oligos (nucleoside phosphorothioates) are isoelectronic analogs of an oligonucleotide (0-oligo) in which a nonbridging oxygen atom of the phosphate group is replaced by a sulfur atom. The S-oligos of the present invention can be prepared by treatment of the corresponding O-oligos with 3H-1,2-benzodithiol-3-one-1,1-dioxide, which is a sulfur transfer reagent. See Iyer, R. P. et al, J. Org. Chem. 55:4693-4698 (1990); and Iyer, R. P. et al., J. Am. Chem. Soc. 112:1253-1254 (1990). Additional 98P7C3 antisense oligonucleotides of the present invention include morpholino antisense oligonucleotides known in the art (see, e.g., Partridge et al., 1996, Antisense & Nucleic Acid Drug Development 6: 169-175).

The 98P7C3 antisense oligonucleotides of the present invention typically can be RNA or DNA that is complementary to and stably hybridizes with the first 100 5′ codons or last 100 3′ codons of the 98P7C3 genomic sequence or the corresponding mRNA. Absolute complementarity is not required, although high degrees of complementarity are preferred. Use of an oligonucleotide complementary to this region allows for the selective hybridization to 98P7C3 mRNA and not to mRNA specifying other regulatory subunits of protein kinase. In one embodiment, 98P7C3 antisense oligonucleotides of the present invention are 15 to 30-mer fragments of the antisense DNA molecule that have a sequence that hybridizes to 98P7C3 mRNA. Optionally, 98P7C3 antisense oligonucleotide is a 30-mer oligonucleotide that is complementary to a region in the first 10 5′ codons or last 10 3′ codons of 98P7C3. Alternatively, the antisense molecules are modified to employ ribozymes in the inhibition of 98P7C3 expression, see, e.g., L. A. Couture & D. T. Stinchcomb; Trends Genet 12: 510-515 (1996).

III.A.3.) Primers and Primer Pairs

Further specific embodiments of this nucleotides of the invention include primers and primer pairs, which allow the specific amplification of polynucleotides of the invention or of any specific parts thereof, and probes that selectively or specifically hybridize to nucleic acid molecules of the invention or to any part thereof. Probes can be labeled with a detectable marker, such as, for example, a radioisotope, fluorescent compound, bioluminescent compound, a chemiluminescent compound, metal chelator or enzyme. Such probes and primers are used to detect the presence of a 98P7C3 polynucleotide in a sample and as a means for detecting a cell expressing a 98P7C3 protein.

Examples of such probes include polypeptides comprising all or part of the human 98P7C3 cDNA sequences shown in FIG. 2. Examples of primer pairs capable of specifically amplifying 98P7C3 mRNAs are also described in the Examples. As will be understood by the skilled artisan, a great many different primers and probes can be prepared based on the sequences provided herein and used effectively to amplify and/or detect a 98P7C3 mRNA.

The 98P7C3 polynucleotides of the invention are useful for a variety of purposes, including but not limited to their use as probes and primers for the amplification and/or detection of the 98P7C3 gene(s), mRNA(s), or fragments thereof, as reagents for the diagnosis and/or prognosis of prostate cancer and other cancers; as coding sequences capable of directing the expression of 98P7C3 polypeptides; as tools for modulating or inhibiting the expression of the 98P7C3 gene(s) and/or translation of the 98P7C3 transcript(s); and as therapeutic agents.

III.A.4.) Isolation of 98P7C3-Encoding Nucleic Acid Molecules

The 98P7C3 cDNA sequences described herein enable the isolation of other polynucleotides encoding 98P7C3 gene product(s), as well as the isolation of polynucleotides encoding 98P7C3 gene product homologs, alternatively spliced isoforms, allelic variants, and mutant forms of the 98P7C3 gene product as well as polynucleotides that encode analogs of 98P7C3-related proteins. Various molecular cloning methods that can be employed to isolate fall length cDNAs encoding an 98P7C3 gene are well known (see, for example, Sambrook, J. et al., Molecular Cloning: A Laboratory Manual, 2d edition, Cold Spring Harbor Press, New York, 1989; Current Protocols in Molecular Biology. Ausubel et al., Eds., Wiley and Sons, 1995). For example, lambda phage cloning methodologies can be conveniently employed, using commercially available cloning systems (e.g., Lambda ZAP Express, Stratagene). Phage clones containing 98P7C3 gene cDNAs can be identified by probing with a labeled 98P7C3 cDNA or a fragment thereof. For example, in one embodiment, the 98P7C3 cDNA (FIG. 2) or a portion thereof can be synthesized and used as a probe to retrieve overlapping and full-length cDNAs corresponding to a 98P7C3 gene. The 98P7C3 gene itself can be isolated by screening genomic DNA libraries, bacterial artificial chromosome libraries (BACs), yeast artificial chromosome libraries (YACs), and the like, with 98P7C3 DNA probes or primers.

III.A.5.) Recombinant Nucleic Acid Molecules and Host-Vector Systems

The invention also provides recombinant DNA or RNA molecules containing an 98P7C3 polynucleotide, fragment, analog or homologue thereof, including but not limited to phages, plasmids, phagemids, cosmids, YACs, BACs, as well as various viral and non-viral vectors well known in the art, and cells transformed or transfected with such recombinant DNA or RNA molecules. Methods for generating such molecules are well known (see, for example, Sambrook et al, 1989, supra).

The invention further provides a host-vector system comprising a recombinant DNA molecule containing a 98P7C3 polynucleotide, fragment, analog or homologue thereof within a suitable prokaryotic or eukaryotic host cell. Examples of suitable eukaryotic host cells include a yeast cell, a plant cell, or an animal cell, such as a mammalian cell or an insect cell (e.g., a baculovirus-infectible cell such as an Sf9 or HighFive cell). Examples of suitable mammalian cells include various prostate cancer cell lines such as DU145 and TsuPr1, other transfectable or transducible prostate cancer cell lines, primary cells (PrEC), as well as a number of mammalian cells routinely used for the expression of recombinant proteins (e.g., COS, CHO, 293, 293T cells). More particularly, a polynucleotide comprising the coding sequence of 98P7C3 or a fragment, analog or homolog thereof can be used to generate 98P7C3 proteins or fragments thereof using any number of host-vector systems routinely used and widely known in the art.

A wide range of host-vector systems suitable for the expression of 98P7C3 proteins or fragments thereof are available, see for example, Sambrook et al., 1989, supra; Current Protocols in Molecular Biology, 1995, supra). Preferred vectors for mammalian expression include but are not limited to pcDNA 3.1 myc-His-tag (Invitrogen) and the retroviral vector pSRαtkneo (Muller et al., 1991, MCB 11:1785). Using these expression vectors, 98P7C3 can be expressed in several prostate cancer and non-prostate cell lines, including for example 293, 293T, rat-1, NIH 3T3 and TsuPr1. The host-vector systems of the invention are useful for the production of a 98P7C3 protein or fragment thereof. Such host-vector systems can be employed to study the functional properties of 98P7C3 and 98P7C3 mutations or analogs.

Recombinant human 98P7C3 protein or an analog or homolog or fragment thereof can be produced by mammalian cells transfected with a construct encoding a 98P7C3-related nucleotide. For example, 293T cells can be transfected with an expression plasmid encoding 98P7C3 or fragment, analog or homolog thereof, the 98P7C3 or related protein is expressed in the 293T cells, and the recombinant 98P7C3 protein is isolated using standard purification methods (e.g., affinity purification using anti-98P7C3 antibodies). In another embodiment, a 98P7C3 coding sequence is subcloned into the retroviral vector pSRαMSVtkneo and used to infect various mammalian cell lines, such as NIH 3T3, TsuPr1, 293 and rat-1 in order to establish 98P7C3 expressing cell lines. Various other expression systems well known in the art can also be employed. Expression constructs encoding a leader peptide joined in frame to the 98P7C3 coding sequence can be used for the generation of a secreted form of recombinant 98P7C3 protein.

As discussed herein, redundancy in the genetic code permits variation in 98P7C3 gene sequences. In particular, it is known in the art that specific host species often have specific codon preferences, and thus one can adapt the disclosed sequence as preferred for a desired host. For example, preferred analog codon sequences typically have rare codons (i.e., codons having a usage frequency of less than about 20% in known sequences of the desired host) replaced with higher frequency codons. Codon preferences for a specific species are calculated, for example, by utilizing codon usage tables available on the INTERNET such as: http://www.dna.affrc.go.jp/˜nakamura/codon.html.

Additional sequence modifications are known to enhance protein expression in a cellular host. These include elimination of sequences encoding spurious polyadenylation signals, exon/intron splice site signals, transposon-like repeats, and/or other such well-characterized sequences that are deleterious to gene expression. The GC content of the sequence is adjusted to levels average for a given cellular host, as calculated by reference to known genes expressed in the host cell. Where possible, the sequence is modified to avoid predicted hairpin secondary mRNA structures. Other useful modifications include the addition of a translational initiation consensus sequence at the start of the open reading frame, as described in Kozak, Mol. Cell Biol., 9:5073-5080 (1989). Skilled artisans understand that the general rule that eukaryotic ribosomes initiate translation exclusively at the 5′ proximal AUG codon is abrogated only under rare conditions (see, e.g., Kozak PNAS 92(7): 2662-2666, (1995) and Kozak NAR 15(20): 8125-8148 (1987)).

IV.) 98P7C3-related Proteins

Another aspect of the present invention provides 98P7C3-related proteins. Specific embodiments of 98P7C3 proteins comprise a polypeptide having all or part of the amino acid sequence of human 98P7C3 as shown in FIG. 2. Alternatively, embodiments of 98P7C3 proteins comprise variant, homolog or analog polypeptides that have alterations in the amino acid sequence of 98P7C3 shown in FIG. 2.

In general, naturally occurring allelic variants of human 98P7C3 share a high degree of structural identity and homology (e.g., 90% or more homology). Typically, allelic variants of the 98P7C3 protein contain conservative amino acid substitutions within the 98P7C3 sequences described herein or contain a substitution of an amino acid from a corresponding position in a homologue of 98P7C3. One class of 98P7C3 allelic variants are proteins that share a high degree of homology with at least a small region of a particular 98P7C3 amino acid sequence, but further contain a radical departure from the sequence, such as a non-conservative substitution, truncation, insertion or frame shift. In comparisons of protein sequences, the terms, similarity, identity, and homology each have a distinct meaning as appreciated in the field of genetics. Moreover, orthology and paralogy can be important concepts describing the relationship of members of a given protein family in one organism to the members of the same family in other organisms.

Amino acid abbreviations are provided in Table II. Conservative amino acid substitutions can frequently be made in a protein without altering either the conformation or the function of the protein. Proteins of the invention can comprise 1, 2, 3, 4, 5, 6, 7, 8, 9, 10, 11, 12, 13, 14, 15 conservative substitutions. Such changes include substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments (see, e.g. Table III herein; pages 13-15 “Biochemistry” 2^{nd} ED. Lubert Stryer ed (Stanford University); Henikoff et al., PNAS 1992 Vol 89 10915-10919; Lei et al., J Biol Chem May 19, 1995; 270(20):11882-6).

Embodiments of the invention disclosed herein include a wide variety of art-accepted variants or analogs of 98P7C3 proteins such as polypeptides having amino acid insertions, deletions and substitutions. 98P7C3 variants can be made using methods known in the art such as site-directed mutagenesis, alanine scanning, and PCR mutagenesis. Site-directed mutagenesis (Carter et al., Nucl. Acids Res., 13:4331 (1986); Zoller et al., Nucl. Acids Res., 10:6487 (1987)), cassette mutagenesis (Wells et al., Gene, 34:315 (1985)), restriction selection mutagenesis (Wells et al., Philos. Trans. R. Soc. London Ser A, 317:415 (1986)) or other known techniques can be performed on the cloned DNA to produce the 98P7C3 variant DNA.

Scanning amino acid analysis can also be employed to identify one or more amino acids along a contiguous sequence that is involved in a specific biological activity such as a protein-protein interaction. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant. Alanine is also typically preferred because it is the most common amino acid. Further, it is frequently found in both buried and exposed positions (Creighton, The Proteins, (W. H. Freeman & Co., N.Y.); Chothia, J. Mol. Biol., 150:1 (1976)). If alanine substitution does not yield adequate amounts of variant, an isosteric amino acid can be used.

As defined herein, 98P7C3 variants, analogs or homologs, have the distinguishing attribute of having at least one epitope that is “cross reactive” with a 98P7C3 protein having the amino acid sequence of SEQ ID NO: 2. As used in this sentence, “cross reactive” means that an antibody or T cell that specifically binds to an 98P7C3 variant also specifically binds to the 98P7C3 protein having the amino acid sequence of SEQ ID NO: 2. A polypeptide ceases to be a variant of the protein shown in SEQ ID NO: 2 when it no longer contains any epitope capable of being recognized by an antibody or T cell that specifically binds to the 98P7C3 protein. Those skilled in the art understand that antibodies that recognize proteins bind to epitopes of varying size, and a grouping of the order of about four or five amino acids, contiguous or not, is regarded as a typical number of amino acids in a minimal epitope. See, e.g., Nair et al., J. Immunol 2000 165(12): 6949-6955; Hebbes et al., Mol Immunol (1989) 26(9):865-73; Schwartz et al., J Immunol (1985) 135(4):2598-608.

Another class of 98P7C3-related protein variants share 70%, 75%, 80%, 85% or 90% or more similarity with the amino acid sequence of SEQ ID NO: 2 or a fragment thereof. Another specific class of 98P7C3 protein variants or analogs comprise one or more of the 98P7C3 biological motifs described herein or presently known in the art. Thus, encompassed by the present invention are analogs of 98P7C3 fragments (nucleic or amino acid) that have altered functional (e.g. immunogenic) properties relative to the starting fragment. It is to be appreciated that motifs now or which become part of the art are to be applied to the nucleic or amino acid sequences of FIG. 2.

As discussed herein, embodiments of the claimed invention include polypeptides containing less than the 532 amino acid sequence of the 98P7C3 protein shown in FIG. 2. For example, representative embodiments of the invention comprise peptides/proteins having any 4, 5, 6, 7, 8, 9, 10, 11, 12, 13, 14, 15 or more contiguous amino acids of the 98P7C3 protein shown in FIG. 2 (SEQ ID NO: 2).

Moreover, representative embodiments of the invention disclosed herein include polypeptides consisting of about amino acid 1 to about amino acid 10 of the 98P7C3 protein shown in FIG. 2 or FIG. 3, polypeptides consisting of about amino acid 10 to about amino acid 20 of the 98P7C3 protein shown in FIG. 2 or FIG. 3, polypeptides consisting of about amino acid 20 to about amino acid 30 of the 98P7C3 protein shown in FIG. 2 or FIG. 3, polypeptides consisting of about amino acid 30 to about amino acid 40 of the 98P7C3 protein shown in FIG. 2 or FIG. 3, polypeptides consisting of about amino acid 40 to about amino acid 50 of the 98P7C3 protein shown in FIG. 2 or FIG. 3, polypeptides consisting of about amino acid 50 to about amino acid 60 of the 98P7C3 protein shown in FIG. 2 or FIG. 3, polypeptides consisting of about amino acid 60 to about amino acid 70 of the 98P7C3 protein shown in FIG. 2 or FIG. 3, polypeptides consisting of about amino acid 70 to about amino acid 80 of the 98P7C3 protein shown in FIG. 2 or FIG. 3, polypeptides consisting of about amino acid 80 to about amino acid 90 of the 98P7C3 protein shown in FIG. 2 or FIG. 3, polypeptides consisting of about amino acid 90 to about amino acid 100 of the 98P7C3 protein shown in FIG. 2 or FIG. 3, etc. throughout the entirety of the 98P7C3 amino acid sequence. Moreover, polypeptides consisting of about amino acid 1 (or 20 or 30 or 40 etc.) to about amino acid 20, (or 130, or 140 or 150 etc.) of the 98P7C3 protein shown in FIG. 2 are embodiments of the invention. It is to be appreciated that the starting and stopping positions in this paragraph refer to the specified position as well as that position plus or minus 5 residues.

98P7C3-related proteins are generated using standard peptide synthesis technology or using chemical cleavage methods well known in the art. Alternatively, recombinant methods can be used to generate nucleic acid molecules that encode a 98P7C3-related protein. In one embodiment, nucleic acid molecules provide a means to generate defined fragments of the 98P7C3 protein (or variants, homologs or analogs thereof).

IV.A.) Motif-bearing Protein Embodiments

Additional illustrative embodiments of the invention disclosed herein include 98P7C3 polypeptides comprising the amino acid residues of one or more of the biological motifs contained within the 98P7C3 polypeptide sequence set forth in FIG. 2 or FIG. 3. Various motifs are known in the art, and a protein can be evaluated for the presence of such motifs by a number of publicly available sites (see, e.g.: http://pfam.wust1.edu/; http://searchlauncher.bcm.tmc.edu/seq-search/struc-predict.html http://psort.ims.u-tokyo.ac.jp/; http://www.cbs.dtu.dk/; http://www.ebi.ac.uk/interpro/scan.html; http://www.expasy.ch/tools/scnpsit1.html; Epimatrix™ and Epimer™, Brown University, http://www.brown.edu/Research/TB-HIV_Lab/epimatrix/epimatrix.html; and BIMAS, http://bimas.dcrt.nih.gov/.). Motif bearing subsequences of the 98P7C3 protein are set forth and identified in Table XIX.

Table XX sets forth several frequently occurring motifs based on pfam searches (http://pfam.wust1.edu/). The columns of Table XX list (1) motif name abbreviation, (2) percent identity found amongst the different member of the motif family, (3) motif name or description and (4) most common function; location information is included if the motif is relevant for location.

Polypeptides comprising one or more of the 98P7C3 motifs discussed above are useful in elucidating the specific characteristics of a malignant phenotype in view of the observation that the 98P7C3 motifs discussed above are associated with growth dysregulation and because 98P7C3 is overexpressed in certain cancers (See, e.g., Table I). Casein kinase II, cAMP and camp-dependent protein kinase, and Protein Kinase C, for example, are enzymes known to be associated with the development of the malignant phenotype (see e.g. Chen et al., Lab Invest., 78(2): 165-174 (1998); Gaiddon et al., Endocrinology 136(10): 4331-4338 (1995); Hall et al., Nucleic Acids Research 24(6): 1119-1126 (1996); Peterziel et al., Oncogene 18(46): 6322-6329 (1999) and O'Brian, Oncol. Rep. 5(2): 305-309 (1998)). Moreover, both glycosylation and myristoylation are protein modifications also associated with cancer and cancer progression (see e.g. Dennis et al., Biochem. Biophys. Acta 1473(1):21-34 (1999); Raju et al., Exp. Cell Res. 235(1): 145-154 (1997)). Amidation is another protein modification also associated with cancer and cancer progression (see e.g. Treston et al., J. Natl. Cancer Inst. Monogr. (13): 169-175 (1992)).

In another embodiment, proteins of the invention comprise one or more of the immunoreactive epitopes identified in accordance with art-accepted methods, such as the peptides set forth in Tables V-XVIII. CTL epitopes can be determined using specific algorithms to identify peptides within an 98P7C3 protein that are capable of optimally binding to specified HLA alleles (e.g., Table IV (A) and Table IV (B); Epimatrix™ and Epimer™, Brown University, http://www.brown.edu/Research/TB-HIV_Lab/epimatrix/epimatrix.html; and BIMAS, http://bimas.dcrt.nih.gov/. Moreover, processes for identifying peptides that have sufficient binding affinity for HLA molecules and which are correlated with being immunogenic epitopes, are well known in the art, and are carried out without undue experimentation. In addition, processes for identifying peptides that are immunogenic epitopes, are well known in the art, and are carried out without undue experimentation either in vitro or in vivo.

Also known in the art are principles for creating analogs of such epitopes in order to modulate immunogenicity. For example, one begins with an epitope that bears a CTL or HTL motif (see, e.g., the HLA Class I motifs or Table IV (A) and the HTL motif of Table IV (B)). The epitope is analoged by substituting out an amino acid at one of the specified positions, and replacing it with another amino acid specified for that position.

A variety of references reflect the art regarding the identification and generation of epitopes in a protein of interest as well as analogs thereof. See, for example, WO 9733602 to Chesnut et al.; Sette, Immunogenetics 1999 50(3-4): 201-212; Sette et al., J. Immunol. 2001 166(2): 1389-1397; Sidney et al., Hum. Immunol. 1997 58(1): 12-20; Kondo et al., Immunogenetics 1997 45(4): 249-258; Sidney et al., J. Immunol. 1996 157(8): 3480-90; and Falk et al., Nature 351: 290-6 (1991); Hunt et al., Science 255:1261-3 (1992); Parker et al., J. Immunol. 149:3580-7 (1992); Parker et al., J. Immunol. 152:163-75 (1994)); Kast et al., 1994 152(8): 3904-12; Borras-Cuesta et al., Hum. Immunol. 2000 61(3): 266-278; Alexander et al., J. Immunol. 2000 164(3); 164(3): 1625-1633; Alexander et al., PMID: 7895164, UI: 95202582; O'Sullivan et al., J. Immunol. 1991 147(8): 2663-2669; Alexander et al., Immunity 1994 1(9): 751-761 and Alexander et al., Immunol. Res. 1998 18(2): 79-92.

Related embodiments of the inventions include polypeptides comprising combinations of the different motifs set forth in Table XIX, and/or, one or more of the predicted CTL epitopes of Table V through Table XVIII, and/or, one or more of the T cell binding motifs known in the art. Preferred embodiments contain no insertions, deletions or substitutions either within the motifs or the intervening sequences of the polypeptides. In addition, embodiments which include a number of either N-terminal and/or C-terminal amino acid residues on either side of these motifs may be desirable (to, for example, include a greater portion of the polypeptide architecture in which the motif is located). Typically the number of N-terminal and/or C-terminal amino acid residues on either side of a motif is between about 1 to about 100 amino acid residues, preferably 5 to about 50 amino acid residues.

98P7C3-related proteins are embodied in many forms, preferably in isolated form A purified 98P7C3 protein molecule will be substantially free of other proteins or molecules that impair the binding of 98P7C3 to antibody, T cell or other ligand. The nature and degree of isolation and purification will depend on the intended use. Embodiments of a 98P7C3-related proteins include purified 98P7C3-related proteins and functional, soluble 98P7C3-related proteins. In one embodiment, a functional, soluble 98P7C3 protein or fragment thereof retains the ability to be bound by antibody, T cell or other ligand.

The invention also provides 98P7C3 proteins comprising biologically active fragments of the 98P7C3 amino acid sequence shown in FIG. 2. Such proteins exhibit properties of the 98P7C3 protein, such as the ability to elicit the generation of antibodies that specifically bind an epitope associated with the 98P7C3 protein; to be bound by such antibodies; to elicit the activation of HTL or CTL; and/or, to be recognized by HTL or CTL.

98P7C3-related polypeptides that contain particularly interesting structures can be predicted and/or identified using various analytical techniques well known in the art, including, for example, the methods of Chou-Fasman, Garnier-Robson, Kyte-Doolittle, Eisenberg, Karplus-Schultz or Jameson-Wolf analysis, or on the basis of immunogenicity. Fragments that contain such structures are particularly useful in generating subunit-specific anti-98P7C3 antibodies, or T cells or in identifying cellular factors that bind to 98P7C3.

CTL epitopes can be determined using specific algorithms to identify peptides within an 98P7C3 protein that are capable of optimally binding to specified HLA alleles (e.g., Table IV (A) and Table IV (B); Epimatrix™ and Epimer™, Brown University (http://www.brown.edu/Research/TB-HIV_Lab/epimatrix/epimatrix.html); and BIMAS, http://bimas.dcrt.nih.gov/). Illustrating this, peptide epitopes from 98P7C3 that are presented in the context of human MHC class I molecules HLA-A1, A2, A3, A11, A24, B7 and B35 were predicted (Tables V-XVIII). Specifically, the complete amino acid sequence of the 98P7C3 protein was entered into the HLA Peptide Motif Search algorithm found in the Bioinformatics and Molecular Analysis Section (BIMAS) web site listed above. The HLA peptide motif search algorithm was developed by Dr. Ken Parker based on binding of specific peptide sequences in the groove of HLA Class I molecules and specifically HLA-A2 (see, e.g., Falk et al., Nature 351: 290-6 (1991); Hunt et al., Science 255:1261-3 (1992); Parker et al., J. Immunol. 149:3580-7 (1992); Parker et al., J. Immunol. 152:163-75 (1994)). This algorithm allows location and ranking of 8-mer, 9-mer, and 10-mer peptides from a complete protein sequence for predicted binding to HLA-A2 as well as numerous other HLA Class I molecules. Many HLA class I binding peptides are 8-, 9-, 10 or 1 1-mers. For example, for class I HLA-A2, the epitopes preferably contain a leucine (L) or methionine (M) at position 2 and a valine (V) or leucine (L) at the C-terminus (see, e.g., Parker et al., J. Immunol. 149:3580-7 (1992)). Selected results of 98P7C3 predicted binding peptides are shown in Tables V-XVIII herein. In Tables V-XVIII, the top 50 ranking candidates, 9-mers and 10-mers, for each family member are shown along with their location, the amino acid sequence of each specific peptide, and an estimated binding score. The binding score corresponds to the estimated half-time of dissociation of complexes containing the peptide at 37° C. at pH 6.5. Peptides with the highest binding score are predicted to be the most tightly bound to HLA Class I on the cell surface for the greatest period of time and thus represent the best immunogenic targets for T-cell recognition.

Actual binding of peptides to an HLA allele can be evaluated by stabilization of HLA expression on the antigen-processing defective cell line T2 (see, e.g., Xue et al., Prostate 30:73-8 (1997) and Peshwa et al., Prostate 36:129-38 (1998)). Immunogenicity of specific peptides can be evaluated in vitro by stimulation of CD8+ cytotoxic T lymphocytes (CTL) in the presence of antigen presenting cells such as dendritic cells.

It is to be appreciated that every epitope predicted by the BIMAS site, Epimer™ and Epimatrix™ sites, or specified by the HLA class I or class I motifs available in the art or which become part of the art such as set forth in Table IV (A) and Table IV (B) are to be “applied” to the 98P7C3 protein. As used in this context “applied” means that the 98P7C3 protein is evaluated, e.g., visually or by computer-based patterns finding methods, as appreciated by those of skill in the relevant art. Every subsequence of the 98P7C3 of 8, 9, 10, or 11 amino acid residues that bears an HLA Class I motif, or a subsequence of 9 or more amino acid residues that bear an HLA Class II motif are within the scope of the invention.

IV.B.) Expression of 98P7C3-related Proteins

In an embodiment described in the examples that follow, 98P7C3 can be conveniently expressed in cells (such as 293T cells) transfected with a commercially available expression vector such as a CMV-driven expression vector encoding 98P7C3 with a C-terminal 6XHis and MYC tag (pcDNA3.1/mycHIS, Invitrogen or Tag5, GenHunter Corporation, Nashville, Tenn.). The TagS vector provides an IgGK secretion signal that can be used to facilitate the production of a secreted 98P7C3 protein in transfected cells. The secreted HIS-tagged 98P7C3 in the culture media can be purified, e.g., using a nickel column using standard techniques.

IV.C.) Modifications of 98P7C3-related Proteins

Modifications of 98P7C3-related proteins such as covalent modifications are included within the scope of this invention. One type of covalent modification includes reacting targeted amino acid residues of a 98P7C3 polypeptide with an organic derivatizing agent that is capable of reacting with selected side chains or the N- or C-terminal residues of the 98P7C3. Another type of covalent modification of the 98P7C3 polypeptide included within the scope of this invention comprises altering the native glycosylation pattern of a protein of the invention. Another type of covalent modification of 98P7C3 comprises linking the 98P7C3 polypeptide to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337.

The 98P7C3-related proteins of the present invention can also be modified to form a chimeric molecule comprising 98P7C3 fused to another, heterologous polypeptide or amino acid sequence. Such a chimeric molecule can be synthesized chemically or recombinantly. A chimeric molecule can have a protein of the invention fused to another tumor-associated antigen or fragment thereof. Alternatively, a protein in accordance with the invention can comprise a fusion of fragments of the 98P7C3 sequence (amino or nucleic acid) such that a molecule is created that is not, through its length, directly homologous to the amino or nucleic acid sequences respectively of FIG. 2. Such a chimeric molecule can comprise multiples of the same subsequence of 98P7C3. A chimeric molecule can comprise a fusion of a 98P7C3-related protein with a polyhistidine epitope tag, which provides an epitope to which immobilized nickel can selectively bind, with cytokines or with growth factors. The epitope tag is generally placed at the amino- or carboxyl- terminus of the 98P7C3. In an alternative embodiment, the chimeric molecule can comprise a fusion of a 98P7C3-related protein with an immunoglobulin or a particular region of an immunoglobulin. For a bivalent form of the chimeric molecule (also referred to as an “immunoadhesin”), such a fusion could be to the Fc region of an IgG molecule. The Ig fusions preferably include the substitution of a soluble (transmembrane domain deleted or inactivated) form of a 98P7C3 polypeptide in place of at least one variable region within an Ig molecule. In a preferred embodiment, the immunoglobulin fusion includes the hinge, CH2 and CH3, or the hinge, CHI, CH2 and CH3 regions of an IgGI molecule. For the production of immunoglobulin fusions see, e.g., U.S. Pat. No. 5,428,130 issued Jun. 27, 1995.

IV.D.) Uses of 98P7C3-related Proteins

The proteins of the invention have a number of different specific uses. As 98P7C3 is highly expressed in prostate and other cancers, 98P7C3-related proteins are used in methods that assess the status of 98P7C3 gene products in normal versus cancerous tissues, thereby elucidating the malignant phenotype. Typically, polypeptides from specific regions of the 98P7C3 protein are used to assess the presence of perturbations (such as deletions, insertions, point mutations etc.) in those regions (such as regions containing one or more motifs). Exemplary assays utilize antibodies or T cells targeting 98P7C3-related proteins comprising the amino acid residues of one or more of the biological motifs contained within the 98P7C3 polypeptide sequence in order to evaluate the characteristics of this region in normal versus cancerous tissues or to elicit an immune response to the epitope. Alternatively, 98P7C3-related proteins that contain the amino acid residues of one or more of the biological motifs in the 98P7C3 protein are used to screen for factors that interact with that region of 98P7C3.

98P7C3 protein fragments/subsequences are particularly useful in generating and characterizing domain-specific antibodies (e.g., antibodies recognizing an extracellular or intracellular epitope of an 98P7C3 protein), for identifying agents or cellular factors that bind to 98P7C3 or a particular structural domain thereof, and in various therapeutic and diagnostic contexts, including but not limited to diagnostic assays, cancer vaccines and methods of preparing such vaccines.

Proteins encoded by the 98P7C3 genes, or by analogs, homologs or fragments thereof, have a variety of uses, including but not limited to generating antibodies and in methods for identifying ligands and other agents and cellular constituents that bind to an 98P7C3 gene product. Antibodies raised against an 98P7C3 protein or fragment thereof are useful in diagnostic and prognostic assays, and imaging methodologies in the management of human cancers characterized by expression of 98P7C3 protein, such as those listed in Table I. Such antibodies can be expressed intracellularly and used in methods of treating patients with such cancers. 98P7C3-related nucleic acids or proteins are also used in generating HTL or CTL responses.

Various immunological assays useful for the detection of 98P7C3 proteins are used, including but not limited to various types of radioimmunoassays, enzyme-linked immunosorbent assays (ELISA), enzyme-linked immunofluorescent assays (ELIFA), immunocytochemical methods, and the like. Antibodies can be labeled and used as immunological imaging reagents capable of detecting 98P7C3- expressing cells (e.g., in radioscintigraphic imaging methods). 98P7C3 proteins are also particularly useful in generating cancer vaccines, as further described herein.

V.) 98P7C3 Antibodies

Another aspect of the invention provides antibodies that bind to 98P7C3-related proteins. Preferred antibodies specifically bind to a 98P7C3-related protein and do not bind (or bind weakly) to peptides or proteins that are not 98P7C3-related proteins. For example, antibodies bind 98P7C3 can bind 98P7C3-related proteins such as the homologs or analogs thereof

98P7C3 antibodies of the invention are particularly useful in prostate cancer diagnostic and prognostic assays, and imaging methodologies. Similarly, such antibodies are useful in the treatment, diagnosis, and/or prognosis of other cancers, to the extent 98P7C3 is also expressed or overexpressed in these other cancers. Moreover, intracellularly expressed antibodies (e.g., single chain antibodies) are therapeutically useful in treating cancers in which the expression of 98P7C3 is involved, such as advanced or metastatic prostate cancers.

The invention also provides various immunological assays useful for the detection and quantification of 98P7C3 and mutant 98P7C3-related proteins. Such assays can comprise one or more 98P7C3 antibodies capable of recognizing and binding a 98P7C3-related protein, as appropriate. These assays are performed within various immunological assay formats well known in the art, including but not limited to various types of radioimmunoassays, enzyme-linked immunosorbent assays (ELISA), enzyme-linked immunofluorescent assays (ELIFA), and the like.

Immunological non-antibody assays of the invention also comprise T cell immunogenicity assays (inhibitory or stimulatory) as well as major histocompatibility complex (MHC) binding assays.

In addition, immunological imaging methods capable of detecting prostate cancer and other cancers expressing 98P7C3 are also provided by the invention, including but not limited to radioscintigraphic imaging methods using labeled 98P7C3 antibodies. Such assays are clinically useful in the detection, monitoring, and prognosis of 98P7C3 expressing cancers such as prostate cancer.

98P7C3 antibodies are also used in methods for purifying a 98P7C3-related protein and for isolating 98P7C3 homologues and related molecules. For example, a method of purifying a 98P7C3-related protein comprises incubating an 98P7C3 antibody, which has been coupled to a solid matrix, with a lysate or other solution containing a 98P7C3-related protein under conditions that permit the 98P7C3 antibody to bind to the 98P7C3-related protein; washing the solid matrix to eliminate impurities; and eluting the 98P7C3-related protein from the coupled antibody. Other uses of the 98P7C3 antibodies of the invention include generating anti-idiotypic antibodies that mimic the 98P7C3 protein.

Various methods for the preparation of antibodies are well known in the art. For example, antibodies can be prepared by immunizing a suitable mammalian host using a 98P7C3-related protein, peptide, or fragment, in isolated or immunoconjugated form (Antibodies: A Laboratory Manual, CSH Press, Eds., Harlow, and Lane (1988); Harlow, Antibodies, Cold Spring Harbor Press, NY (1989)). In addition, fusion proteins of 98P7C3 can also be used, such as a 98P7C3 GST-fusion protein. In a particular embodiment, a GST fusion protein comprising all or most of the amino acid sequence of FIG. 2 or FIG. 3 is produced, then used as an immunogen to generate appropriate antibodies. In another embodiment, a 98P7C3-related protein is synthesized and used as an immunogen.

In addition, naked DNA immunization techniques known in the art are used (with or without purified 98P7C3-related protein or 98P7C3 expressing cells) to generate an immune response to the encoded inmmunogen (for review, see Donnelly et al., 1997, Ann. Rev. Immunol. 15: 617-648).

The amino acid sequence of 98P7C3 as shown in FIG. 2 or FIG. 3 can be analyzed to select specific regions of the 98P7C3 protein for generating antibodies. For example, hydrophobicity and hydrophilicity analyses of the 98P7C3 amino acid sequence are used to identify hydrophilic regions in the 98P7C3 structure. Regions of the 98P7C3 protein that show immunogenic structure, as well as other regions and domains, can readily be identified using various other methods known in the art, such as Chou-Fasman, Garnier-Robson, Kyte-Doolittle, Eisenberg, Karplus-Schultz or Jameson-Wolf analysis. Thus, each region identified by any of these programs or methods is within the scope of the present invention. Methods for the generation of 98P7C3 antibodies are further illustrated by way of the examples provided herein. Methods for preparing a protein or polypeptide for use as an immunogen are well known in the art. Also well known in the art are methods for preparing immunogenic conjugates of a protein with a carrier, such as BSA, KLH or other carrier protein. In some circumstances, direct conjugation using, for example, carboxamide reagents are used; in other instances linking reagents such as those supplied by Pierce Chemical Co., Rockford, Ill., are effective. Administration of a 98P7C3 immunogen is often conducted by injection over a suitable time period and with use of a suitable adjuvant, as is understood in the art. During the immunization schedule, titers of antibodies can be taken to determine adequacy of antibody formation.

98P7C3 monoclonal antibodies can be produced by various means well known in the art. For example, immortalized cell lines that secrete a desired monoclonal antibody are prepared using the standard hybridoma technology of Kohler and Milstein or modifications that immortalize antibody-producing B cells, as is generally known. Immortalized cell lines that secrete the desired antibodies are screened by immunoassay in which the antigen is a 98P7C3-related protein. When the appropriate immortalized cell culture is identified, the cells can be expanded and antibodies produced either from in vitro cultures or from ascites fluid.

The antibodies or fragments of the invention can also be produced, by recombinant means. Regions that bind specifically to the desired regions of the 98P7C3 protein can also be produced in the context of chimeric or complementarity determining region (CDR) grafted antibodies of multiple species origin. Humanized or human 98P7C3 antibodies can also be produced, and are preferred for use in therapeutic contexts. Methods for humanizing murine and other non-human antibodies, by substituting one or more of the non-human antibody CDRs for corresponding human antibody sequences, are well known (see for example, Jones et al., 1986, Nature 321: 522-525; Riechrmann et al., 1988, Nature 332: 323-327; Verhoeyen et al., 1988, Science 239: 1534-1536). See also, Carter et al., 1993, Proc. Natl. Acad. Sci. USA 89: 4285 and Sims et al., 1993, J. Immunol. 151: 2296.

Methods for producing fully human monoclonal antibodies include phage display and transgenic methods (for review, see Vaughan et al., 1998, Nature Biotechnology 16: 535-539). Fully human 98P7C3 monoclonal antibodies can be generated using cloning technologies employing large human Ig gene combinatorial libraries (i.e., phage display) (Griffiths and Hoogenboom, Building an in vitro immune system: human antibodies from phage display libraries. In: Protein Engineering of Antibody Molecules for Prophylactic and Therapeutic Applications in Man, Clark, M. (Ed.), Nottingham Academic, pp 45-64 (1993); Burton and Barbas, Human Antibodies from combinatorial libraries. Id., pp 65-82). Fully human 98P7C3 monoclonal antibodies can also be produced using transgenic mice engineered to contain human immunoglobulin gene loci as described in PCT Patent Application WO98/24893, Kucherlapati and Jakobovits et al., published Dec. 3, 1997 (see also, Jakobovits, 1998, Exp. Opin. Invest. Drugs 7(4): 607-614; U.S. Pat. Nos. 6,162,963 issued Dec. 19, 2000; 6,150,584 issued Nov. 12, 2000; and, 6,114,598 issued Sep. 5, 2000). This method avoids the in vitro manipulation required with phage display technology and efficiently produces high affinity authentic human antibodies.

Reactivity of 98P7C3 antibodies with an 98P7C3-related protein can be established by a number of well known means, including Western blot, immunoprecipitation, ELISA, and FACS analyses using, as appropriate, 98P7C3-related proteins, 98P7C3-expressing cells or extracts thereof. A 98P7C3 antibody or fragment thereof can be labeled with a detectable marker or conjugated to a second molecule. Suitable detectable markers include, but are not limited to, a radioisotope, a fluorescent compound, a bioluminescent compound, chemiluminescent compound, a metal chelator or an enzyme. Further, bi-specific antibodies specific for two or more 98P7C3 epitopes are generated using methods generally known in the art. Homodimeric antibodies can also be generated by cross-linking techniques known in the art (e.g., Wolff et al., Cancer Res. 53: 2560-2565).

VI.) 98P7C3 Transgenic Animals

Nucleic acids that encode a 98P7C3-related protein can also be used to generate either transgenic animals or “knock out” animals which, in turn, are useful in the development and screening of therapeutically useful reagents. In accordance with established techniques, cDNA encoding 98P7C3 can be used to clone genomic DNA that encodes 98P7C3. The cloned genomic sequences can then be used to generate transgenic animals containing cells that express DNA that encode 98P7C3. Methods for generating transgenic animals, particularly animals such as mice or rats, have become conventional in the art and are described, for example, in U.S. Pat. Nos. 4,736,866 issued Apr. 12, 1988, and 4,870,009 issued Sep. 26, 1989. Typically, particular cells would be targeted for 98P7C3 transgene incorporation with tissue-specific enhancers.

Transgenic animals that include a copy of a transgene encoding 98P7C3 can be used to examine the effect of increased expression of DNA that encodes 98P7C3. Such animals can be used as tester animals for reagents thought to confer protection from, for example, pathological conditions associated with its overexpression. In accordance with this aspect of the invention, an animal is treated with a reagent and a reduced incidence of a pathological condition, compared to untreated animals that bear the transgene, would indicate a potential therapeutic intervention for the pathological condition.

Alternatively, non-human homologues of 98P7C3 can be used to construct a 98P7C3 “knock out” animal that has a defective or altered gene encoding 98P7C3 as a result of homologous recombination between the endogenous gene encoding 98P7C3 and altered genomic DNA encoding 98P7C3 introduced into an embryonic cell of the animal. For example, cDNA that encodes 98P7C3 can be used to clone genomic DNA encoding 98P7C3 in accordance with established techniques. A portion of the genomic DNA encoding 98P7C3 can be deleted or replaced with another gene, such as a gene encoding a selectable marker that can be used to monitor integration. Typically, several kilobases of unaltered flanking DNA (both at the 5′ and 3′ ends) are included in the vector (see, e.g., Thomas and Capecchi, Cell, 51:503 (1987) for a description of homologous recombination vectors). The vector is introduced into an embryonic stem cell line (e.g., by electroporation) and cells in which the introduced DNA has homologously recombined with the endogenous DNA are selected (see, e.g.,, Li et al., Cell, 69:915 (1992)). The selected cells are then injected into a blastocyst of an animal (e.g., a mouse or rat) to form aggregation chimeras (see, e.g.,, Bradley, in Teratocarcinomas and Embryonic Stem Cells: A Practical Approach, E. J. Robertson, ed. (IRL, Oxford, 1987), pp. 113-152). A chimeric embryo can then be implanted into a suitable pseudopregnant female foster animal, and the embryo brought to term to create a “knock out” animal. Progeny harboring the homologously recombined DNA in their germ cells can be identified by standard techniques and used to breed animals in which all cells of the animal contain the homologously recombined DNA. Knock out animals can be characterized, for example, for their ability to defend against certain pathological conditions or for their development of pathological conditions due to absence of the 98P7C3 polypeptide.

VII.) Methods for the Detection of 98P7C3

Another aspect of the present invention relates to methods for detecting 98P7C3 polynucleotides and 98P7C3-related proteins, as well as methods for identifying a cell that expresses 98P7C3. The expression profile of 98P7C3 makes it a diagnostic marker for metastasized disease. Accordingly, the status of 98P7C3 gene products provides information useful for predicting a variety of factors including susceptibility to advanced stage disease, rate of progression, and/or tumor aggressiveness. As discussed in detail herein, the status of 98P7C3 gene products in patient samples can be analyzed by a variety protocols that are well known in the art including immunohistochemical analysis, the variety of Northern blotting techniques including in situ hybridization, RT-PCR analysis (for example on laser capture micro-dissected samples), Western blot analysis and tissue array analysis.

More particularly, the invention provides assays for the detection of 98P7C3 polynucleotides in a biological sample, such as serum, bone, prostate, and other tissues, urine, semen, cell preparations, and the like. Detectable 98P7C3 polynucleotides include, for example, a 98P7C3 gene or fragment thereof, 98P7C3 mRNA, alternative splice variant 98P7C3 mRNAs, and recombinant DNA or RNA molecules that contain a 98P7C3 polynucleotide. A number of methods for amplifying and/or detecting the presence of 98P7C3 polynucleotides are well known in the art and can be employed in the practice of this aspect of the invention.

In one embodiment, a method for detecting an 98P7C3 mRNA in a biological sample comprises producing cDNA from the sample by reverse transcription using at least one primer; amplifying the cDNA so produced using an 98P7C3 polynucleotides as sense and antisense primers to amplify 98P7C3 cDNAs therein; and detecting the presence of the amplified 98P7C3 cDNA. Optionally, the sequence of the amplified 98P7C3 cDNA can be determined.

In another embodiment, a method of detecting a 98P7C3 gene in a biological sample comprises first isolating genomic DNA from the sample; amplifying the isolated genomic DNA using 98P7C3 polynucleotides as sense and antisense primers; and detecting the presence of the amplified 98P7C3 gene. Any number of appropriate sense and antisense probe combinations can be designed from the nucleotide sequence provided for the 98P7C3 (FIG. 2) and used for this purpose.

The invention also provides assays for detecting the presence of an 98P7C3 protein in a tissue or other biological sample such as serum, semen, bone, prostate, urine, cell preparations, and the like. Methods for detecting a 98P7C3-related protein are also well known and include, for example, immunoprecipitation, immunohistochemical analysis, Western blot analysis, molecular binding assays, ELISA, ELIFA and the like. For example, a method of detecting the presence of a 98P7C3-related protein in a biological sample comprises first contacting the sample with a 98P7C3 antibody, a 98P7C3-reactive fragment thereof, or a recombinant protein containing an antigen binding region of a 98P7C3 antibody; and then detecting the binding of 98P7C3-related protein in the sample.

Methods for identifying a cell that expresses 98P7C3 are also within the scope of the invention. In one embodiment, an assay for identifying a cell that expresses a 98P7C3 gene comprises detecting the presence of 98P7C3 mRNA in the cell. Methods for the detection of particular mRNAs in cells are well known and include, for example, hybridization assays using complementary DNA probes (such as in situ hybridization using labeled 98P7C3 riboprobes, Northern blot and related techniques) and various nucleic acid amplification assays (such as RT-PCR using complementary primers specific for 98P7C3, and other amplification type detection methods, such as, for example, branched DNA, SISBA, TMA and the like). Alternatively, an assay for identifying a cell that expresses a 98P7C3 gene comprises detecting the presence of 98P7C3-related protein in the cell or secreted by the cell. Various methods for the detection of proteins are well known in the art and are employed for the detection of 98P7C3-related proteins and cells that express 98P7C3-related proteins.

98P7C3 expression analysis is also useful as a tool for identifying and evaluating agents that modulate 98P7C3 gene expression. For example, 98P7C3 expression is significantly upregulated in prostate cancer, and is expressed in cancers of the tissues listed in Table I. Identification of a molecule or biological agent that inhibits 98P7C3 expression or over-expression in cancer cells is of therapeutic value. For example, such an agent can be identified by using a screen that quantifies 98P7C3 expression by RT-PCR, nucleic acid hybridization or antibody binding.

VIII.) Methods for Monitoring the Status of 98P7C3-related Genes and Their Products

Oncogenesis is known to be a multistep process where cellular growth becomes progressively dysregulated and cells progress from a normal physiological state to precancerous and then cancerous states (see, e.g., Alers et al., Lab Invest. 77(5): 437-438 (1997) and Isaacs et al., Cancer Surv. 23:19-32 (1995)). In this context, examining a biological sample for evidence of dysregulated cell growth (such as aberrant 98P7C3 expression in cancers) allows for early detection of such aberrant physiology, before a pathologic state such as cancer has progressed to a stage that therapeutic options are more limited and or the prognosis is worse. In such examinations, the status of 98P7C3 in a biological sample of interest can be compared, for example, to the status of 98P7C3 in a corresponding normal sample (e.g. a sample from that individual or alternatively another individual that is not affected by a pathology). An alteration in the status of 98P7C3 in the biological sample (as compared to the normal sample) provides evidence of dysregulated cellular growth. In addition to using a biological sample that is not affected by a pathology as a normal sample, one can also use a predetermined normative value such as a predetermined normal level of mRNA expression (see, e.g., Grever et al., J. Comp. Neurol. December 1996 9;376(2):306-14 and U.S. Patent No. 5,837,501) to compare 98P7C3 status in a sample.

The term “status” in this context is used according to its art accepted meaning and refers to the condition or state of a gene and its products. Typically, skilled artisans use a number of parameters to evaluate the condition or state of a gene and its products. These include, but are not limited to the location of expressed gene products (including the location of 98P7C3 expressing cells) as well as the level, and biological activity of expressed gene products (such as 98P7C3 mRNA, polynucleotides and polypeptides). Typically, an alteration in the status of 98P7C3 comprises a change in the location of 98P7C3 and/or 98P7C3 expressing cells and/or an increase in 98P7C3 mRNA and/or protein expression.

98P7C3 status in a sample can be analyzed by a number of means well known in the art, including without limitation, immunohistochemical analysis, in situ hybridization, RT-PCR analysis on laser capture micro-dissected samples, Western blot analysis, and tissue array analysis. Typical protocols for evaluating the status of the 98P7C3 gene and gene products are found, for example in Ausubel et al. eds., 1995, Current Protocols In Molecular Biology, Units 2 (Northern Blotting), 4 (Southern Blotting), 15 (Immunoblotting) and 18 (PCR Analysis). Thus, the status of 98P7C3 in a biological sample is evaluated by various methods utilized by skilled artisans including, but not limited to genomic Southern analysis (to examine, for example perturbations in the 98P7C3 gene), Northern analysis and/or PCR analysis of 98P7C3 mRNA (to examine, for example alterations in the polynucleotide sequences or expression levels of 98P7C3 mRNAs), and, Western and/or immunohistochemical analysis (to examine, for example alterations in polypeptide sequences, alterations in polypeptide localization within a sample, alterations in expression levels of 98P7C3 proteins and/or associations of 98P7C3 proteins with polypeptide binding partners). Detectable 98P7C3 polynucleotides include, for example, a 98P7C3 gene or fragment thereof, 98P7C3 mRNA, alternative splice variants, 98P7C3 mRNAs, and recombinant DNA or RNA molecules containing a 98P7C3 polynucleotide.

The expression profile of 98P7C3 makes it a diagnostic marker for local and/or metastasized disease, and provides information on the growth or oncogenic potential of a biological sample. In particular, the status of 98P7C3 provides information useful for predicting susceptibility to particular disease stages, progression, and/or tumor aggressiveness. The invention provides methods and assays for determining 98P7C3 status and diagnosing cancers that express 98P7C3, such as cancers of the tissues listed in Table I. For example, because 98P7C3 mRNA is so highly expressed in prostate and other cancers relative to normal prostate tissue, assays that evaluate the levels of 98P7C3 mRNA transcripts or proteins in a biological sample can be used to diagnose a disease associated with 98P7C3 dysregulation, and can provide prognostic information useful in defining appropriate therapeutic options.

The expression status of 98P7C3 provides information including the presence, stage and location of dysplastic, precancerous and cancerous cells, predicting susceptibility to various stages of disease, and/or for gauging tumor aggressiveness. Moreover, the expression profile makes it useful as an imaging reagent for metastasized disease. Consequently, an aspect of the invention is directed to the various molecular prognostic and diagnostic methods for examining the status of 98P7C3 in biological samples such as those from individuals suffering from, or suspected of suffering from a pathology characterized by dysregulated cellular growth, such as cancer.

As described above, the status of 98P7C3 in a biological sample can be examined by a number of well-known procedures in the art. For example, the status of 98P7C3 in a biological sample taken from a specific location in the body can be examined by evaluating the sample for the presence or absence of 98P7C3 expressing cells (e.g. those that express 98P7C3 mRNAs or proteins). This examination can provide evidence of dysregulated cellular growth, for example, when 98P7C3-expressing cells are found in a biological sample that does not normally contain such cells (such as a lymph node), because such alterations in the status of 98P7C3 in a biological sample are often associated with dysregulated cellular growth. Specifically, one indicator of dysregulated cellular growth is the metastases of cancer cells from an organ of origin (such as the prostate) to a different area of the body (such as a lymph node). In this context, evidence of dysregulated cellular growth is important for example because occult lymph node metastases can be detected in a substantial proportion of patients with prostate cancer, and such metastases are associated with known predictors of disease progression (see, e.g., Murphy et al., Prostate 42(4): 315-317 (2000); Su et al., Semin. Surg. Oncol. 18(1):17-28 (2000) and Freeman et al., J Urol August 1995 154(2 Pt 1):474-8).

In one aspect, the invention provides methods for monitoring 98P7C3 gene products by determining the status of 98P7C3 gene products expressed by cells from an individual suspected of having a disease associated with dysregulated cell growth (such as hyperplasia or cancer) and then comparing the status so determined to the status of 98P7C3 gene products in a corresponding normal sample. The presence of aberrant 98P7C3 gene products in the test sample relative to the normal sample provides an indication of the presence of dysregulated cell growth within the cells of the individual.

In another aspect, the invention provides assays useful in determining the presence of cancer in an individual, comprising detecting a significant increase in 98P7C3 mRNA or protein expression in a test cell or tissue sample relative to expression levels in the corresponding normal cell or tissue. The presence of 98P7C3 mRNA can, for example, be evaluated in tissue samples including but not limited to those listed in Table I. The presence of significant 98P7C3 expression in any of these tissues is useful to indicate the emergence, presence and/or severity of a cancer, since the corresponding normal tissues do not express 98P7C3 mRNA or express it at lower levels.

In a related embodiment, 98P7C3 status is determined at the protein level rather than at the nucleic acid level. For example, such a method comprises determining the level of 98P7C3 protein expressed by cells in a test tissue sample and comparing the level so determined to the level of 98P7C3 expressed in a corresponding normal sample. In one embodiment, the presence of 98P7C3 protein is evaluated, for example, using immunohistochemical methods. 98P7C3 antibodies or binding partners capable of detecting 98P7C3 protein expression are used in a variety of assay formats well known in the art for this purpose.

In a further embodiment, one can evaluate the status 98P7C3 nucleotide and amino acid sequences in a biological sample in order to identify perturbations in the structure of these molecules. These perturbations can include insertions, deletions, substitutions and the like. Such evaluations are useful because perturbations in the nucleotide and amino acid sequences are observed in a large number of proteins associated with a growth dysregulated phenotype (see, e.g., Marrogi et al., 1999, J. Cutan. Pathol. 26(8):369-378). For example, a mutation in the sequence of 98P7C3 may be indicative of the presence or promotion of a tumor. Such assays therefore have diagnostic and predictive value where a mutation in 98P7C3 indicates a potential loss of function or increase in tumor growth.

A wide variety of assays for observing perturbations in nucleotide and amino acid sequences are well known in the art. For example, the size and structure of nucleic acid or amino acid sequences of 98P7C3 gene products are observed by the Northern, Southern, Western, PCR and DNA sequencing protocols discussed herein. In addition, other methods for observing perturbations in nucleotide and amino acid sequences such as single strand conformation polymorphism analysis are well known in the art (see, e.g., U.S. Pat. Nos. 5,382,510 issued Sep. 7, 1999, and 5,952,170 issued Jan. 17, 1995).

Additionally, one can examine the methylation status of the 98P7C3 gene in a biological sample. Aberrant demethylation and/or hypermethylation of CpG islands in gene 5′ regulatory regions frequently occurs in immortalized and transformed cells, and can result in altered expression of various genes. For example, promoter hypermethylation of the pi-class glutathione S-transferase (a protein expressed in normal prostate but not expressed in >90% of prostate carcinomas) appears to permanently silence transcription of this gene and is the most frequently detected genomic alteration in prostate carcinomas (De Marzo et al., Am. J. Pathol. 155(6): 1985-1992 (1999)). In addition, this alteration is present in at least 70% of cases of high-grade prostatic intraepithelial neoplasia (PIN) (Brooks et al, Cancer Epidemiol. Biomarkers Prev., 1998, 7:531-536). In another example, expression of the LAGE-I tumor specific gene (which is not expressed in normal prostate but is expressed in 25-50% of prostate cancers) is induced by deoxy-azacytidine in lymphoblastoid cells, suggesting that tumoral expression is due to demethylation (Lethe et al., Int. J. Cancer 76(6): 903-908 (1998)). A variety of assays for examining methylation status of a gene are well known in the art. For example, one can utilize, in Southern hybridization approaches, methylation-sensitive restriction enzymes which cannot cleave sequences that contain methylated CpG sites to assess the methylation status of CpG islands. In addition, MSP (methylation specific PCR) can rapidly profile the methylation status of all the CpG sites present in a CpG island of a given gene. This procedure involves initial modification of DNA by sodium bisulfite (which will convert all unmethylated cytosines to uracil) followed by amplification using primers specific for methylated versus unmethylated DNA. Protocols involving methylation interference can also be found for example in Current Protocols In Molecular Biology, Unit 12, Frederick M. Ausubel et al. eds., 1995.

Gene amplification is an additional method for assessing the status of 98P7C3. Gene amplification is measured in a sample directly, for example, by conventional Southern blotting or Northern blotting to quantitate the transcription of mRNA (Thomas, 1980, Proc. Natl. Acad. Sci. USA, 77:5201-5205), dot blotting (DNA analysis), or in situ hybridization, using an appropriately labeled probe, based on the sequences provided herein. Alternatively, antibodies are employed that recognize specific duplexes, including DNA duplexes, RNA duplexes, and DNA-RNA hybrid duplexes or DNA-protein duplexes. The antibodies in turn are labeled and the assay carried out where the duplex is bound to a surface, so that upon the formation of duplex on the surface, the presence of antibody bound to the duplex can be detected.

Biopsied tissue or peripheral blood can be conveniently assayed for the presence of cancer cells using for example, Northern, dot blot or RT-PCR analysis to detect 98P7C3 expression. The presence of RT-PCR amplifiable 98P7C3 mRNA provides an indication of the presence of cancer. RT-PCR assays are well known in the art. RT-PCR detection assays for tumor cells in peripheral blood are currently being evaluated for use in the diagnosis and management of a number of human solid tumors. In the prostate cancer field, these include RT-PCR assays for the detection of cells expressing PSA and PSM (Verkaik et al., 1997, Urol. Res. 25:373-384; Ghossein et al., 1995, J. Clin. Oncol. 13:1195-2000; Heston et al., 1995, Clin. Chem. 41:1687-1688).

A further aspect of the invention is an assessment of the susceptibility that an individual has for developing cancer. In one embodiment, a method for predicting susceptibility to cancer comprises detecting 98P7C3 mRNA or 98P7C3 protein in a tissue sample, its presence indicating susceptibility to cancer, wherein the degree of 98P7C3 mRNA expression correlates to the degree of susceptibility. In a specific embodiment, the presence of 98P7C3 in prostate or other tissue is examined, with the presence of 98P7C3 in the sample providing an indication of prostate cancer susceptibility (or the emergence or existence of a prostate tumor). Similarly, one can evaluate the integrity 98P7C3 nucleotide and amino acid sequences in a biological sample, in order to identify perturbations in the structure of these molecules such as insertions, deletions, substitutions and the like. The presence of one or more perturbations in 98P7C3 gene products in the sample is an indication of cancer susceptibility (or the emergence or existence of a tumor).

The invention also comprises methods for gauging tumor aggressiveness. In one embodiment, a method for gauging aggressiveness of a tumor comprises determining the level of 98P7C3 mRNA or 98P7C3 protein expressed by tumor cells, comparing the level so determined to the level of 98P7C3 mRNA or 98P7C3 protein expressed in a corresponding normal tissue taken from the same individual or a normal tissue reference sample, wherein the degree of 98P7C3 mRNA or 98P7C3 protein expression in the tumor sample relative to the normal sample indicates the degree of aggressiveness. In a specific embodiment, aggressiveness of a tumor is evaluated by determining the extent to which 98P7C3 is expressed in the tumor cells, with higher expression levels indicating more aggressive tumors. Another embodiment is the evaluation of the integrity of 98P7C3 nucleotide and amino acid sequences in a biological sample, in order to identify perturbations in the structure of these molecules such as insertions, deletions, substitutions and the like. The presence of one or more perturbations indicates more aggressive tumors.

Another embodiment of the invention is directed to methods for observing the progression of a malignancy in an individual over time. In one embodiment, methods for observing the progression of a malignancy in an individual over time comprise determining the level of 98P7C3 mRNA or 98P7C3 protein expressed by cells in a sample of the tumor, comparing the level so determined to the level of 98P7C3 mRNA or 98P7C3 protein expressed in an equivalent tissue sample taken from the same individual at a different time, wherein the degree of 98P7C3 mRNA or 98P7C3 protein expression in the tumor sample over time provides information on the progression of the cancer. In a specific embodiment, the progression of a cancer is evaluated by determining 98P7C3 expression in the tumor cells over time, where increased expression over time indicates a progression of the cancer. Also, one can evaluate the integrity 98P7C3 nucleotide and amino acid sequences in a biological sample in order to identify perturbations in the structure of these molecules such as insertions, deletions, substitutions and the like, where the presence of one or more perturbations indicates a progression of the cancer.

The above diagnostic approaches can be combined with any one of a wide variety of prognostic and diagnostic protocols known in the art. For example, another embodiment of the invention is directed to methods for observing a coincidence between the expression of 98P7C3 gene and 98P7C3 gene products (or perturbations in 98P7C3 gene and 98P7C3 gene products) and a factor that is associated with malignancy, as a means for diagnosing and prognosticating the status of a tissue sample. A wide variety of factors associated with malignancy can be utilized, such as the expression of genes associated with malignancy (e.g. PSA, PSCA and PSM expression for prostate cancer etc.) as well as gross cytological observations (see, e.g., Bocking et al., 1984, Anal. Quant. Cytol. 6(2):74-88; Epstein, 1995, Hum. Pathol. 26(2):223-9; Thorson et al., 1998, Mod. Pathol. 11(6):543-51; Baisden et al., 1999, Am. J. Surg. Pathol. 23(8):918-24). Methods for observing a coincidence between the expression of 98P7C3 gene and 98P7C3 gene products (or perturbations in 98P7C3 gene and 98P7C3 gene products) and another factor that is associated with malignancy are useful, for example, because the presence of a set of specific factors that coincide with disease provides information crucial for diagnosing and prognosticating the status of a tissue sample.

In one embodiment, methods for observing a coincidence between the expression of 98P7C3 gene and 98P7C3 gene products (or perturbations in 98P7C3 gene and 98P7C3 gene products) and another factor associated with malignancy entails detecting the overexpression of 98P7C3 mRNA or protein in a tissue sample, detecting the overexpression of PSA mRNA or protein in a tissue sample (or PSCA or PSM expression), and observing a coincidence of 98P7C3 mRNA or protein and PSA mRNA or protein overexpression (or PSCA or PSM expression). In a specific embodiment, the expression of 98P7C3 and PSA mRNA in prostate tissue is examined, where the coincidence of 98P7C3 and PSA mRNA overexpression in the sample indicates the existence of prostate cancer, prostate cancer susceptibility or the emergence or status of a prostate tumor.

Methods for detecting and quantifying the expression of 98P7C3 mRNA or protein are described herein, and standard nucleic acid and protein detection and quantification technologies are well known in the art. Standard methods for the detection and quantification of 98P7C3 mRNA include in situ hybridization using labeled 98P7C3 riboprobes, Northern blot and related techniques using 98P7C3 polynucleotide probes, RT-PCR analysis using primers specific for 98P7C3, and other amplification type detection methods, such as, for example, branched DNA, SISBA, TMA and the like. In a specific embodiment, semi-quantitative RT-PCR is used to detect and quantify 98P7C3 mRNA expression. Any number of primers capable of amplifying 98P7C3 can be used for this purpose, including but not limited to the various primer sets specifically described herein. In a specific embodiment, polyclonal or monoclonal antibodies specifically reactive with the wild-type 98P7C3 protein can be used in an immunohistochemical assay of biopsied tissue.

IX.) Identification of Molecules That Interact With 98P7C3

The 98P7C3 protein and nucleic acid sequences disclosed herein allow a skilled artisan to identify proteins, small molecules and other agents that interact with 98P7C3, as well as pathways activated by 98P7C3 via any one of a variety of art accepted protocols. For example, one can utilize one of the so-called interaction trap systems (also referred to as the “two-hybrid assay”). In such systems, molecules interact and reconstitute a transcription factor which directs expression of a reporter gene, whereupon the expression of the reporter gene is assayed. Other systems identify protein-protein interactions in vivo through reconstitution of a eukaryotic transcriptional activator, see, e.g., U.S. Pat. Nos. 5,955,280 issued Sep. 21, 1999, 5,925,523 issued Jul. 20, 1999, 5,846,722 issued Dec. 8, 1998 and 6,004,746 issued Dec. 21, 1999. Algorithms are also available in the art for genome-based predictions of protein function (see, e.g., Marcotte, et al., Nature 402: Nov. 4, 1999, 83-86).

Alternatively one can screen peptide libraries to identify molecules that interact with 98P7C3 protein sequences. In such methods, peptides that bind to a molecule such as 98P7C3 are identified by screening libraries that encode a random or controlled collection of amino acids. Peptides encoded by the libraries are expressed as fusion proteins of bacteriophage coat proteins, the bacteriophage particles are then screened against the protein of interest.

Accordingly, peptides having a wide variety of uses, such as therapeutic, prognostic or diagnostic reagents, are thus identified without any prior information on the structure of the expected ligand or receptor molecule. Typical peptide libraries and screening methods that can be used to identify molecules that interact with 98P7C3 protein sequences are disclosed for example in U.S. Pat. Nos. 5,723,286 issued Mar. 3, 1998 and 5,733,731 issued Mar. 31, 1998.

Alternatively, cell lines that express 98P7C3 are used to identify protein-protein interactions mediated by 98P7C3. Such interactions can be examined using immunoprecipitation techniques (see, e.g., Hamilton B J, et al. Biochem. Biophys. Res. Commun. 1999, 261:646-51). 98P7C3 protein can be immunoprecipitated from 98P7C3-expressing cell lines using anti-98P7C3 antibodies. Alternatively, antibodies against His-tag can be used in a cell line engineered to express 98P7C3 (vectors mentioned above). The immunoprecipitated complex can be examined for protein association by procedures such as Western blotting, ³⁵S-methionine labeling of proteins, protein microsequencing, silver staining and two-dimensional gel electrophoresis.

Small molecules and ligands that interact with 98P7C3 can be identified through related embodiments of such screening assays. For example, small molecules can be identified that interfere with protein function, including molecules that interfere with 98P7C3's ability to mediate phosphorylation and de-phosphorylation, interaction with DNA or RNA molecules as an indication of regulation of cell cycles, second messenger signaling or tumorigenesis. Similarly, small molecules that modulate ion channel, protein pump, or cell communication function of 98P7C3 are identified and used to treat patients that have a cancer that expresses the 98P7C3 antigen (see, e.g., Hille, B., Ionic Channels of Excitable Membranes 2^{nd} Ed., Sinauer Assoc., Sunderland, Mass., 1992). Moreover, ligands that regulate 98P7C3 function can be identified based on their ability to bind 98P7C3 and activate a reporter construct. Typical methods are discussed for example in U.S. Pat. No. 5,928,868 issued Jul. 27, 1999, and include methods for forming hybrid ligands in which at least one ligand is a small molecule. In an illustrative embodiment, cells engineered to express a fusion protein of 98P7C3 and a DNA-binding protein are used to co-express a fusion protein of a hybrid ligand/small molecule and a cDNA library transcriptional activator protein. The cells further contain a reporter gene, the expression of which is conditioned on the proximity of the first and second fusion proteins to each other, an event that occurs only if the hybrid ligand binds to target sites on both hybrid proteins. Those cells that express the reporter gene are selected and the unknown small molecule or the unknown ligand is identified. This method provides a means of identifying both activators and inhibitors of 98P7C3.

An embodiment of this invention comprises a method of screening for a molecule that interacts with an 98P7C3 amino acid sequence shown in FIG. 2 and FIG. 3 (SEQ ID NO: 2), comprising the steps of contacting a population of molecules with the 98P7C3 amino acid sequence, allowing the population of molecules and the 98P7C3 amino acid sequence to interact under conditions that facilitate an interaction, determining the presence of a molecule that interacts with the 98P7C3 amino acid sequence, and then separating molecules that do not interact with the 98P7C3 amino acid sequence from molecules that do. In a specific embodiment, the method further comprises purifying a molecule that interacts with the 98P7C3 amino acid sequence. The identified molecule can be used to modulate a function performed by 98P7C3. In a preferred embodiment, the 98P7C3 amino acid sequence is contacted with a library of peptides.

X.) Therapeutic Methods and Compositions

The identification of 98P7C3 as a protein that is normally expressed in a restricted set of tissues, but which is also expressed in prostate and other cancers, opens a number of therapeutic approaches to the treatment of such cancers. As discussed herein, it is possible that 98P7C3 functions as a transcription factor involved in activating tumor-promoting genes or repressing genes that block tumorigenesis.

Accordingly, therapeutic approaches that inhibit the activity of the 98P7C3 protein are useful for patients suffering from a cancer that expresses 98P7C3. These therapeutic approaches generally fall into two classes. One class comprises various methods for inhibiting the binding or association of the 98P7C3 protein with its binding partner or with other proteins. Another class comprises a variety of methods for inhibiting the transcription of the 98P7C3 gene or translation of 98P7C3 mRNA.

X.A.) 98P7C3 as a Target for Antibody-Based Therapy

98P7C3 is an attractive target for antibody-based therapeutic strategies. A number of antibody strategies are known in the art for targeting both extracellular and intracellular molecules (see, e.g., complement and ADCC mediated killing as well as the use of intrabodies). Because 98P7C3 is expressed by cancer cells of various lineages and not by corresponding normal cells, systemic administration of 98P7C3-immunoreactive compositions are prepared that exhibit excellent sensitivity without toxic, non-specific and/or non-target effects caused by binding of the immunoreactive composition to non-target organs and tissues. Antibodies specifically reactive with domains of 98P7C3 are useful to treat 98P7C3-expressing cancers systemically, either as conjugates with a toxin or therapeutic agent, or as naked antibodies capable of inhibiting cell proliferation or function.

98P7C3 antibodies can be introduced into a patient such that the antibody binds to 98P7C3 and modulates a function, such as an interaction with a binding partner, and consequently mediates destruction of the tumor cells and/or inhibits the growth of the tumor cells. Mechanisms by which such antibodies exert a therapeutic effect can include complement-mediated cytolysis, antibody-dependent cellular cytotoxicity, modulation of the physiological function of 98P7C3, inhibition of ligand binding or signal transduction pathways, modulation of tumor cell differentiation, alteration of tumor angiogenesis factor profiles, and/or apoptosis.

Those skilled in the art understand that antibodies can be used to specifically target and bind immunogenic molecules such as an immunogenic region of the 98P7C3 sequence shown in FIG. 2. In addition, skilled artisans understand that it is routine to conjugate antibodies to cytotoxic agents. When cytotoxic and/or therapeutic agents are delivered directly to cells, such as by conjugating them to antibodies specific for a molecule expressed by that cell (e.g. 98P7C3), the cytotoxic agent will exert its known biological effect (i.e. cytotoxicity) on those cells.

A wide variety of compositions and methods for using antibody-cytotoxic agent conjugates to kill cells are known in the art. In the context of cancers, typical methods entail administering to an animal having a tumor a biologically effective amount of a conjugate comprising a selected cytotoxic and/or therapeutic agent linked to a targeting agent (e.g. an anti-98P7C3 antibody) that binds to a marker (e.g. 98P7C3) expressed, accessible to binding or localized on the cell surfaces. A typical embodiment is a method of delivering a cytotoxic and/or therapeutic agent to a cell expressing 98P7C3, comprising conjugating the cytotoxic agent to an antibody that immunospecifically binds to a 98P7C3 epitope, and, exposing the cell to the antibody-agent conjugate. Another illustrative embodiment is a method of treating an individual suspected of suffering from metastasized cancer, comprising a step of administering parenterally to said individual a pharmaceutical composition comprising a therapeutically effective amount of an antibody conjugated to a cytotoxic and/or therapeutic agent.

Cancer immunotherapy using anti-98P7C3 antibodies can be done in accordance with various approaches that have been successfully employed in the treatment of other types of cancer, including but not limited to colon cancer (Arlen et al., 1998, Crit. Rev. Immunol. 18:133-138), multiple myeloma (Ozaki et al., 1997, Blood 90:3179-3186, Tsunenari et al., 1997, Blood 90:2437-2444), gastric cancer (Kasprzyk et al., 1992, Cancer Res. 52:2771-2776), B-cell lymphoma (Funakoshi et al., 1996, J. Immunother. Emphasis Tumor Immunol. 19:93-101), leukemia (Zhong et al., 1996, Leuk. Res. 20:581-589), colorectal cancer (Moun et al., 1994, Cancer Res. 54:6160-6166; Velders et al., 1995, Cancer Res. 55:4398-4403), and breast cancer (Shepard et al., 1991, J. Clin. Immunol. 11:117-127). Some therapeutic approaches involve conjugation of naked antibody to a toxin, such as the conjugation of Y⁹¹ or I¹³¹ to anti-CD20 antibodies (e.g., Zevalin™, IDEC Pharmaceuticals Corp. or Bexxar™, Coulter Pharmaceuticals), while others involve co-administration of antibodies and other therapeutic agents, such as Herceptin™ (trastuzumab) with paclitaxel (Genentech, Inc.). To treat prostate cancer, for example, 98P7C3 antibodies can be administered in conjunction with radiation, chemotherapy or hormone ablation.

Although 98P7C3 antibody therapy is useful for all stages of cancer, antibody therapy can be particularly appropriate in advanced or metastatic cancers. Treatment with the antibody therapy of the invention is indicated for patients who have received one or more rounds of chemotherapy. Alternatively, antibody therapy of the invention is combined with a chemotherapeutic or radiation regimen for patients who have not received chemotherapeutic treatment. Additionally, antibody therapy can enable the use of reduced dosages of concomitant chemotherapy, particularly for patients who do not tolerate the toxicity of the chemotherapeutic agent very well.

Cancer patients can be evaluated for the presence and level of 98P7C3 expression, preferably using immunohistochemical assessments of tumor tissue, quantitative 98P7C3 imaging, or other techniques that reliably indicate the presence and degree of 98P7C3 expression. Immunohistochemical analysis of tumor biopsies or surgical specimens is preferred for this purpose. Methods for immunohistochernical analysis of tumor tissues are well known in the art.

Anti-98P7C3 monoclonal antibodies that treat prostate and other cancers include those that initiate a potent immune response against the tumor or those that are directly cytotoxic. In this regard, anti-98P7C3 monoclonal antibodies (mAbs) can elicit tumor cell lysis by either complement-mediated or antibody-dependent cell cytotoxicity (ADCC) mechanisms, both of which require an intact Fc portion of the immunoglobulin molecule for interaction with effector cell Fc receptor sites on complement proteins. In addition, anti-98P7C3 mAbs that exert a direct biological effect on tumor growth are useful to treat cancers that express 98P7C3. Mechanisms by which directly cytotoxic mAbs act include: inhibition of cell growth, modulation of cellular differentiation, modulation of tumor angiogenesis factor profiles, and the induction of apoptosis. The mechanism(s) by which a particular anti-98P7C3 mAb exerts an anti-tumor effect is evaluated using any number of in vitro assays that evaluate cell death such as ADCC, ADMMC, complement-mediated cell lysis, and so forth, as is generally known in the art.

In some patients, the use of murine or other non-human monoclonal antibodies, or human/mouse chimeric mAbs can induce moderate to strong immune responses against the non-human antibody. This can result in clearance of the antibody from circulation and reduced efficacy. In the most severe cases, such an immune response can lead to the extensive formation of immune complexes which, potentially, can cause renal failure. Accordingly, preferred monoclonal antibodies used in the therapeutic methods of the invention are those that are either fully human or humanized and that bind specifically to the target 98P7C3 antigen with high affinity but exhibit low or no antigenicity in the patient.

Therapeutic methods of the invention contemplate the administration of single anti-98P7C3 mAbs as well as combinations, or cocktails, of different mAbs. Such mAb cocktails can have certain advantages inasmuch as they contain mAbs that target different epitopes, exploit different effector mechanisms or combine directly cytotoxic mabs with mAbs that rely on immune effector functionality. Such mAbs in combination can exhibit synergistic therapeutic effects. In addition, anti-98P7C3 mAbs can be administered concomitantly with other therapeutic modalities, including but not limited to various chemotherapeutic agents, androgen-blockers, immune modulators (e.g., IL-2, GM-CSF), surgery or radiation. The anti-98P7C3 mAbs are administered in their “naked” or unconjugated form, or can have a therapeutic agent(s) conjugated to them.

Anti-98P7C3 antibody formulations are administered via any route capable of delivering the antibodies to a tumor cell. Routes of administration include, but are not limited to, intravenous, intraperitoneal, intramuscular, intratumor, intradermal, and the like. Treatment generally involves repeated administration of the anti-98P7C3 antibody preparation, via an acceptable route of administration such as intravenous injection (IV), typically at a dose in the range of about 0.1 to about 10 mg/kg body weight. In general, doses in the range of 10-500 mg mAb per week are effective and well tolerated.

Based on clinical experience with the Herceptin mAb in the treatment of metastatic breast cancer, an initial loading dose of approximately 4 mg/kg patient body weight IV, followed by weekly doses of about 2 mg/kg IV of the anti-98P7C3 mAb preparation represents an acceptable dosing regimen. Preferably, the initial loading dose is administered as a 90 minute or longer infusion. The periodic maintenance dose is administered as a 30 minute or longer infusion, provided the initial dose was well tolerated. As appreciated by those of skill in the art, various factors can influence the ideal dose regimen in a particular case. Such factors include, for example, the binding affinity and half life of the Ab or mAbs used, the degree of 98P7C3 expression in the patient, the extent of circulating shed 98P7C3 antigen, the desired steady-state antibody concentration level, frequency of treatment, and the influence of chemotherapeutic or other agents used in combination with the treatment method of the invention, as well as the health status of a particular patient.

Optionally, patients should be evaluated for the levels of 98P7C3 in a given sample (e.g. the levels of circulating 98P7C3 antigen and/or 98P7C3 expressing cells) in order to assist in the determination of the most effective dosing regimen, etc. Such evaluations are also used for monitoring purposes throughout therapy, and are useful to gauge therapeutic success in combination with the evaluation of other parameters (such as serum PSA levels in prostate cancer therapy).

X.B.) Anti-Cancer Vaccines

The invention further provides cancer vaccines comprising a 98P7C3-related protein or 98P7C3-related nucleic acid. In view of the expression of 98P7C3, cancer vaccines prevent and/or treat 98P7C3-expressing cancers without creating non-specific effects on non-target tissues. The use of a tumor antigen in a vaccine that generates humoral and/or cell-mediated immune responses as anti-cancer therapy is well known in the art and has been employed in prostate cancer using human PSMA and rodent PAP immunogens (Hodge et al., 1995, Int. J. Cancer 63:231-237; Fong et al., 1997, J. Immunol. 159:3113-3117).

Genetic immunization methods can be employed to generate prophylactic or therapeutic humoral and cellular immune responses directed against cancer cells expressing 98P7C3. Constructs comprising DNA encoding a 98P7C3-related protein/immunogen and appropriate regulatory sequences can be injected directly into muscle or skin of an individual, such that the cells of the muscle or skin take-up the construct and express the encoded 98P7C3 protein/immunogen. Alternatively, a vaccine comprises a 98P7C3-related protein. Expression of the 98P7C3-related protein immunogen results in the generation of prophylactic or therapeutic humoral and cellular immunity against cells that bear 98P7C3 protein. Various prophylactic and therapeutic genetic immunization techniques known in the art can be used (for review, see information and references published at Internet address www.genweb.com).

Such methods can be readily practiced by employing a 98P7C3-related protein, or an 98P7C3-encoding nucleic acid molecule and recombinant vectors capable of expressing and presenting the 98P7C3 immunogen (which typically comprises a number of antibody or T cell epitopes). Skilled artisans understand that a wide variety of vaccine systems for delivery of imnmunoreactive epitopes are known in the art (see, e.g., Heryln et al., Ann Med February 1999 31(1):66-78; Maruyama et al., Cancer Immunol Immunother June 2000 49(3):123-32) Briefly, such methods of generating an immune response (e.g. humoral and/or cell-mediated) in a mammal, comprise the steps of: exposing the mammal's immune system to an immunoreactive epitope (e.g. an epitope present in the 98P7C3 protein shown in SEQ ID NO: 2 or analog or homolog thereof) so that the mammal generates an immune response that is specific for that epitope (e.g. generates antibodies that specifically recognize that epitope). In a preferred method, the 98P7C3 immunogen contains a biological motif.

CTL epitopes can be determined using specific algorithms to identify peptides within 98P7C3 protein that are capable of optimally binding to specified HLA alleles (e.g., Table IV (A) and Table IV (B); Epimer™ and Epimatrix™, Brown University (http://www.brown.edu/Research/TB-HIV_Lab/epimatrix/epimatrix.html); and, BIMAS, (http://bimas.dcrt.nih.gov/). In a preferred embodiment, the 98P7C3 immunogen contains one or more amino acid sequences identified using one of the pertinent analytical techniques well known in the art, such as the sequences shown in Tables V-XVIII or a peptide of 8, 9, 10 or 11 amino acids specified by an HLA Class I motif (e.g., Table IV (A)) and/or a peptide of at least 9 amino acids that comprises an HLA Class II motif (e.g., Table IV (B)). As is appreciated in the art, the HLA Class I binding groove is essentially closed ended so that peptides of only a particular size range can fit into the groove and be bound, generally HLA Class I epitopes are 8, 9, 10, or 11 amino acids long. In contrast, the HLA Class II binding groove is essentially open ended; therefore a peptide of about 9 or more amino acids can be bound by an HLA Class II molecule. Due to the binding groove differences between HLA Class I and II, HLA Class I motifs are length specific, i.e., position two of a Class I motif is the second amino acid in an amino to carboxyl direction of the peptide. The amino acid positions in a Class II motif are relative only to each other, not the overall peptide, i.e., additional amino acids can be attached to the amino and/or carboxyl termini of a motif-bearing sequence. HLA Class II epitopes are often 9, 10, 11, 12, 13, 14, 15, 16, 17, 18, 19, 20, 21, 22, 23, 24, or 25 amino acids long, or longer than 25 amino acids.

A wide variety of methods for generating an immune response in a mammal are known in the art (for example as the first step in the generation of hybridomas). Methods of generating an immune response in a mammal comprise exposing the mammal's immune system to an immunogenic epitope on a protein (e.g. the 98P7C3 protein) so that an immune response is generated. A typical embodiment consists of a method for generating an immune response to 98P7C3 in a host, by contacting the host with a sufficient amount of at least one 98P7C3 B cell or cytotoxic T-cell epitope or analog thereof; and at least one periodic interval thereafter re-contacting the host with the 98P7C3 B cell or cytotoxic T-cell epitope or analog thereof. A specific embodiment consists of a method of generating an immune response against a 98P7C3-related protein or a man-made multiepitopic peptide comprising: administering 98P7C3 immunogen (e.g. the 98P7C3 protein or a peptide fragment thereof, an 98P7C3 fusion protein or analog etc.) in a vaccine preparation to a human or another mammal. Typically, such vaccine preparations further contain a suitable adjuvant (see, e.g., U.S. Pat. No. 6,146,635) or a universal helper epitope such as a PADRE™ peptide (Epimmune Inc., San Diego, Calif.; see, e.g., Alexander et al., J. Immunol. 2000 164(3); 164(3): 1625-1633; Alexander et al., Immunity 1994 1(9): 751-761 and Alexander et al., Immunol. Res. 1998 18(2): 79-92). An alternative method comprises generating an immune response in an individual against a 98P7C3 immunogen by: administering in vivo to muscle or skin of the individual's body a DNA molecule that comprises a DNA sequence that encodes an 98P7C3 immunogen, the DNA sequence operatively linked to regulatory sequences which control the expression of the DNA sequence; wherein the DNA molecule is taken up by cells, the DNA sequence is expressed in the cells and an immune response is generated against the immunogen (see, e.g., U.S. Pat. No. 5,962,428). The DNA can be dissociated from an infectious agent. Optionally a genetic vaccine facilitator such as anionic lipids; saponins; lectins; estrogenic compounds; hydroxylated lower alkyls; dimethyl sulfoxide; and urea is also administered.

Thus, viral gene delivery systems are used to deliver a 98P7C3-related nucleic acid molecule. Various viral gene delivery systems that can be used in the practice of the invention include, but are not limited to, vaccinia, fowlpox, canarypox, adenovirus, influenza, poliovirus, adeno-associated virus, lentivirus, and sindbis virus (Restifo, 1996, Curr. Opin. Immunol. 8:658-663). Non-viral delivery systems can also be employed by introducing naked DNA encoding a 98P7C3-related protein into the patient (e.g., intramuscularly or intradermally) to induce an anti-tumor response. In one embodiment, the full-length human 98P7C3 cDNA is employed. In another enbodiment, 98P7C3 nucleic acid molecules encoding specific cytotoxic T lymphocyte (CTL) and/or antibody epitopes are employed.

Various ex vivo strategies can also be employed to generate an immune response. One approach involves the use of antigen presenting cells (APCs) such as dendritic cells to present 98P7C3 antigen to a patient's immune system. Dendritic cells express MHC class I and II molecules, B7 co-stimulator, and IL-12, and are thus highly specialized antigen presenting cells. In prostate cancer, autologous dendritic cells pulsed with peptides of the prostate-specific membrane antigen (PSMA) are being used in a Phase I clinical trial to stimulate prostate cancer patients' immune systems (Tjoa et al., 1996, Prostate 28:65-69; Murphy et al., 1996, Prostate 29:371-380). Thus, dendritic cells can be used to present 98P7C3 peptides to T cells in the context of MHC class I or II molecules. In one embodiment, autologous dendritic cells are pulsed with 98P7C3 peptides capable of binding to MHC class I and/or class II molecules. In another embodiment, dendritic cells are pulsed with the complete 98P7C3 protein. Yet another embodiment involves engineering the overexpression of the 98P7C3 gene in dendritic cells using various implementing vectors known in the art, such as adenovirus (Arthur et al., 1997, Cancer Gene Ther. 4:17-25), retrovirus (Henderson et al., 1996, Cancer Res. 56:3763-3770), lentivirus, adeno-associated virus, DNA transfection (Ribas et al., 1997, Cancer Res. 57:2865-2869), or tumor-derived RNA transfection (Ashley et al., 1997, J. Exp. Med. 186:1177-1182). Cells that express 98P7C3 can also be engineered to express immune modulators, such as GM-CSF, and used as immunizing agents.

Anti-idiotypic anti-98P7C3 antibodies can also be used in anti-cancer therapy as a vaccine for inducing an immune response to cells expressing a 98P7C3-related protein. In particular, the generation of anti-idiotypic antibodies is well known in the art; this methodology can readily be adapted to generate anti-idiotypic anti-98P7C3 antibodies that mimic an epitope on a 98P7C3-related protein (see, for example, Wagner et al., 1997, Hybridoma 16: 33-40; Foon et al., 1995, J. Clin. Invest. 96:334-342; Herlyn et al., 1996, Cancer Immunol. Immunother. 43:65-76). Such an anti-idiotypic antibody can be used in cancer vaccine strategies.

XI.) Inhibition of 98P7C3 Protein Function

The invention includes various methods and compositions for inhibiting the binding of 98P7C3 to its binding partner or its association with other protein(s) as well as methods for inhibiting 98P7C3 function.

XI.A.) Inhibition of 98P7C3 With Intracellular Antibodies

In one approach, a recombinant vector that encodes single chain antibodies that specifically bind to 98P7C3 are introduced into 98P7C3 expressing cells via gene transfer technologies. Accordingly, the encoded single chain anti-98P7C3 antibody is expressed intracellularly, binds to 98P7C3 protein, and thereby inhibits its function. Methods for engineering such intracellular single chain antibodies are well known. Such intracellular antibodies, also known as “intrabodies”, are specifically targeted to a particular compartment within the cell, providing control over where the inhibitory activity of the treatment is focused. This technology has been successfully applied in the art (for review, see Richardson and Marasco, 1995, TIBTECH vol. 13). Intrabodies have been shown to virtually eliminate the expression of otherwise abundant cell surface receptors (see, e.g., Richardson et al., 1995, Proc. Natl. Acad. Sci. USA 92: 3137-3141; Beerli et al., 1994, J. Biol. Chem. 289: 23931-23936; Deshane et al., 1994, Gene Ther. 1: 332-337).

Single chain antibodies comprise the variable domains of the heavy and light chain joined by a flexible linker polypeptide, and are expressed as a single polypeptide. Optionally, single chain antibodies are expressed as a single chain variable region fragment joined to the light chain constant region. Well-known intracellular trafficking signals are engineered into recombinant polynucleotide vectors encoding such single chain antibodies in order to precisely target the intrabody to the desired intracellular compartment. For example, intrabodies targeted to the endoplasmic reticulum (ER) are engineered to incorporate a leader peptide and, optionally, a C-terminal ER retention signal, such as the KDEL amino acid motif. Intrabodies intended to exert activity in the nucleus are engineered to include a nuclear localization signal. Lipid moieties are joined to intrabodies in order to tether the intrabody to the cytosolic side of the plasma membrane. Intrabodies can also be targeted to exert function in the cytosol. For example, cytosolic intrabodies are used to sequester factors within the cytosol, thereby preventing them from being transported to their natural cellular destination.

In one embodiment, intrabodies are used to capture 98P7C3 in the nucleus, thereby preventing its activity within the nucleus. Nuclear targeting signals are engineered into such 98P7C3 intrabodies in order to achieve the desired targeting. Such 98P7C3 intrabodies are designed to bind specifically to a particular 98P7C3 domain. In another embodiment, cytosolic intrabodies that specifically bind to the 98P7C3 protein are used to prevent 98P7C3 from gaining access to the nucleus, thereby preventing it from exerting any biological activity within the nucleus (e.g., preventing 98P7C3 from forming transcription complexes with other factors).

In order to specifically direct the expression of such intrabodies to particular cells, the transcription of the intrabody is placed under the regulatory control of an appropriate tumor-specific promoter and/or enhancer. In order to target intrabody expression specifically to prostate, for example, the PSA promoter and/or promoter/enhancer can be utilized (See, for example, U.S. Pat. No. 5,919,652 issued Jul. 6, 1999).

XI.B.) Inhibition of 98P7C3 with Recombinant Proteins

In another approach, recombinant molecules bind to 98P7C3 and thereby inhibit 98P7C3 function. For example, these recombinant molecules prevent or inhibit 98P7C3 from accessing/binding to its binding partner(s) or associating with other protein(s). Such recombinant molecules can, for example, contain the reactive part(s) of a 98P7C3 specific antibody molecule. In a particular embodiment, the 98P7C3 binding domain of a 98P7C3 binding partner is engineered into a dimeric fusion protein, whereby the fusion protein comprises two 98P7C3 ligand binding domains linked to the Fc portion of a human IgG, such as human IgG1. Such IgG portion can contain, for example, the C_{H}2 and C_{H}3 domains and the hinge region, but not the C_{H}1 domain. Such dimeric fusion proteins are administered in soluble form to patients suffering from a cancer associated with the expression of 98P7C3, whereby the dimeric fusion protein specifically binds to 98P7C3 and blocks 98P7C3 interaction with a binding partner. Such dimeric fusion proteins are further combined into multimeric proteins using known antibody linking technologies.

XI.C.) Inhibition of 98P7C3 Transcription or Translation

The present invention also comprises various methods and compositions for inhibiting the transcription of the 98P7C3 gene. Similarly, the invention also provides methods and compositions for inhibiting the translation of 98P7C3 mRNA into protein.

In one approach, a method of inhibiting the transcription of the 98P7C3 gene comprises contacting the 98P7C3 gene with a 98P7C3 antisense polynucleotide. In another approach, a method of inhibiting 98P7C3 mRNA translation comprises contacting the 98P7C3 mRNA with an antisense polynucleotide. In another approach, a 98P7C3 specific ribozyme is used to cleave the 98P7C3 message, thereby inhibiting translation. Such antisense and ribozyme based methods can also be directed to the regulatory regions of the 98P7C3 gene, such as the 98P7C3 promoter and/or enhancer elements. Similarly, proteins capable of inhibiting a 98P7C3 gene transcription factor are used to inhibit 98P7C3 mRNA transcription. The various polynucleotides and compositions useful in the aforementioned methods have been described above. The use of antisense and ribozyme molecules to inhibit transcription and translation is well known in the art.

Other factors that inhibit the transcription of 98P7C3 by interfering with 98P7C3 transcriptional activation are also useful to treat cancers expressing 98P7C3. Similarly, factors that interfere with 98P7C3 processing are useful to treat cancers that express 98P7C3. Cancer treatment methods utilizing such factors are also within the scope of the invention.

XI.D.) General Considerations for Therapeutic Strategies

Gene transfer and gene therapy technologies can be used to deliver therapeutic polynucleotide molecules to tumor cells synthesizing 98P7C3 (i.e., antisense, ribozyme, polynucleotides encoding intrabodies and other 98P7C3 inhibitory molecules). A number of gene therapy approaches are known in the art. Recombinant vectors encoding 98P7C3 antisense polynucleotides, ribozymes, factors capable of interfering with 98P7C3 transcription, and so forth, can be delivered to target tumor cells using such gene therapy approaches.

The above therapeutic approaches can be combined with any one of a wide variety of surgical, chemotherapy or radiation therapy regimens. The therapeutic approaches of the invention can enable the use of reduced dosages of chemotherapy (or other therapies) and/or less frequent administration, an advantage for all patients and particularly for those that do not tolerate the toxicity of the chemotherapeutic agent well.

The anti-tumor activity of a particular composition (e.g., antisense, ribozyme, intrabody), or a combination of such compositions, can be evaluated using various in vitro and in vivo assay systems. In vitro assays that evaluate therapeutic activity include cell growth assays, soft agar assays and other assays indicative of tumor promoting activity, binding assays capable of determining the extent to which a therapeutic composition will inhibit the binding of 98P7C3 to a binding partner, etc.

In vivo, the effect of a 98P7C3 therapeutic composition can be evaluated in a suitable animal model. For example, xenogenic prostate cancer models can be used, wherein human prostate cancer explants or passaged xenograft tissues are introduced into immune compromised animals, such as nude or SCID mice (Klein et al., 1997, Nature Medicine 3: 402-408). For example, PCT Patent Application WO98/16628, Sawyers et al., published Apr. 23, 1998, describes various xenograft models of human prostate cancer capable of recapitulating the development of primary tumors, micrometastasis, and the formation of osteoblastic metastases characteristic of late stage disease. Efficacy can be predicted using assays that measure inhibition of tumor formation, tumor regression or metastasis, and the like.

In vivo assays that evaluate the promotion of apoptosis are useful in evaluating therapeutic compositions. In one embodiment, xenografts from tumor bearing mice treated with the therapeutic composition can be examined for the presence of apoptotic foci and compared to untreated control xenograft-bearing mice. The extent to which apoptotic foci are found in the tumors of the treated mice provides an indication of the therapeutic efficacy of the composition.

The therapeutic compositions used in the practice of the foregoing methods can be formulated into pharmaceutical compositions comprising a carrier suitable for the desired delivery method. Suitable carriers include any material that when combined with the therapeutic composition retains the anti-tumor function of the therapeutic composition and is generally non-reactive with the patient's immune system. Examples include, but are not limited to, any of a number of standard pharmaceutical carriers such as sterile phosphate buffered saline solutions, bacteriostatic water, and the like (see, generally, Remington's Pharmaceutical Sciences 16^{th} Edition, A. Osal., Ed., 1980).

Therapeutic formulations can be solubilized and administered via any route capable of delivering the therapeutic composition to the tumor site. Potentially effective routes of administration include, but are not limited to, intravenous, parenteral, intraperitoneal, intramuscular, intratumor, intradermal, intraorgan, orthotopic, and the like. A preferred formulation for intravenous injection comprises the therapeutic composition in a solution of preserved bacteriostatic water, sterile unpreserved water, and/or diluted in polyvinylchloride or polyethylene bags containing 0.9% sterile Sodium Chloride for Injection, USP. Therapeutic protein preparations can be lyophilized and stored as sterile powders, preferably under vacuum, and then reconstituted in bacteriostatic water (containing for example, benzyl alcohol preservative) or in sterile water prior to injection.

Dosages and administration protocols for the treatment of cancers using the foregoing methods will vary with the method and the target cancer, and will generally depend on a number of other factors appreciated in the art.

XII.) Kits

For use in the diagnostic and therapeutic applications described herein, kits are also within the scope of the invention. Such kits can comprise a carrier, package or container that is compartmentalized to receive one or more containers such as vials, tubes, and the like, each of the container(s) comprising one of the separate elements to be used in the method. For example, the container(s) can comprise a probe that is or can be detectably labeled. Such probe can be an antibody or polynucleotide specific for a 98P7C3-related protein or a 98P7C3 gene or message, respectively. Where the method utilizes nucleic acid hybridization to detect the target nucleic acid, the kit can also have containers containing nucleotide(s) for amplification of the target nucleic acid sequence and/or a container comprising a reporter-means, such as a biotin-binding protein, such as avidin or streptavidin, bound to a reporter molecule, such as an enzymatic, florescent, or radioisotope label. The kit can include all or part of the amino acid sequence of FIG. 2 or analogs thereof, or a nucleic acid molecule that encodes such amino acid sequences.

The kit of the invention will typically comprise the container described above and one or more other containers comprising materials desirable from a commercial and user standpoint including buffers, diluents, filters, needles, syringes, and package inserts with instructions for use.

A label can be present on the container to indicate that the composition is used for a specific therapy or non-therapeutic application, and can also indicate directions for either in vivo or in vitro use, such as those described above. Directions and or other information can also be included on an insert which is included with the kit.

The 98P7C3 cDNA has been deposited under the requirements of the Budapest Treaty on Feb. 2, 2000, with the American Type Culture Collection (ATCC), 10801 University Blvd., Manassas, Va. 20110-2209 USA, as plasmid p98P7C3-EBB10, and has been assigned Accession No. PTA-1261.

### EXAMPLES

Various aspects of the invention are further described and illustrated by way of the several examples that follow, none of which are intended to limit the scope of the invention.

### Example 1

### SSH-Generated Isolation of a cDNA Fragment of the 98P7C3 Gene

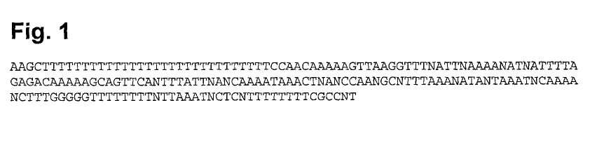

The SSH cDNA fragment 98P7C3 (FIG. 1) was derived from a subtraction utilizing the xenografts LAPC9AD (14 days after castration) minus LAPC9AD (non-castrated mouse). The full-length cDNA clone 98P7C3-Pro-pCR2.1 (FIG. 2) was identified by assembling EST fragments homologous to 98P7C3 into a large contiguous sequence with an ORF and amplifying the ORF by PCR using prostate first strand cDNA.

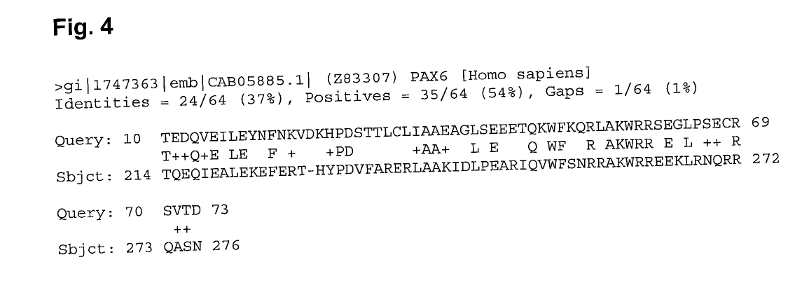

The cDNA clone 98P7C3-Pro-pCR2.1 encodes a 699 amino acid ORF with 10 transmembrane domains predicted at the cell surface (PSORT). The 98P7C3 protein is similar to a GenBank protein AK025164 with one amino acid difference (FIG. 4A). This amino acid difference at amino acid position 689 may be significant since it is located in the long extracellular C-terminal region that may be involved in ligand binding, may affect the stability of the protein, or may be involved in the binding of the 98P7C3 protein to itself or other proteins. Protein AK025164 is novel, it was isolated from normal colon library, and has not been associated with any cancers. In addition, 98P7C3 is homologous to several yeast proteins, one of which is predicted to be localized to the cell surface and involved in sensitivity to certain drugs (FIG. 4B).

Materials and Methods

LAPC Xenografts and Human Tissues:

LAPC xenografts were obtained from Dr. Charles Sawyers (UCLA) and generated as described (Klein et al, 1997, Nature Med. 3: 402-408; Craft et al., 1999, Cancer Res. 59: 5030-5036). Androgen dependent and independent LAPC-4 xenografts LAPC-4 AD and AI, respectively) and LAPC-9 AD and AI xenografts were grown in male SCID mice and were passaged as small tissue chunks in recipient males. LAPC-4 and -9 AI xenografts were derived from LAPC-4 or -9 AD tumors, respectively. To generate the A1 xenografts, male mice bearing AD tumors were castrated and maintained for 2-3 months. After the tumors re-grew, the tumors were harvested and passaged in castrated males or in female SCID mice.

Cell Lines:

Human cell lines (e.g., HeLa) were obtained from the ATCC and were maintained in DMEM with 5% fetal calf serum.

RNA Isolation:

Tumor tissue and cell lines were homogenized in Trizol reagent (Life Technologies, Gibco BRL) using 10 ml/g tissue or 10 ml/10⁸ cells to isolate total RNA. Poly A RNA was purified from total RNA using Qiagen's Oligotex mRNA Mini and Midi kits. Total and mRNA were quantified by spectrophotometric analysis (O.D. 260/280 mn) and analyzed by gel electrophoresis.

Oligonucleotides:

The following HPLC purified oligonucleotides were used.

| | |
|---|---|
| DPNCDN (cDNA synthesis primer): | |
| 5′TTTTGATCAAGCTT₃₀3′ | (SEQ ID NO: 7) |
| Adaptor 1: | |
| 5′CTAATACGACTCACTATAGGGCTCGAGCGGCCGCCCGGGCAG3′ | (SEQ ID NO: 8) |
| 3′GGCCCGTCCTAG5′ | (SEQ ID NO: 9) |
| Adaptor 2: | |
| 5′GTAATACGACTCACTATAGGGCAGCGTGGTCGCGGCCGAG3′ | (SEQ ID NO: 10) |
| 3′CGGCTCCTAG5′ | (SEQ ID NO: 11) |
| PCR primer 1: | |
| 5′CTAATACGACTCACTATAGGGC3′ | (SEQ ID NO: 12) |
| Nested primer (NP)1: | |
| 5′TCGAGCGGCCGCCCGGGCAGGA3′ | (SEQ ID NO: 13) |
| Nested primer (NP)2: | |
| 5′AGCGTGGTCGCGGCCGAGGA3′ | (SEQ ID NO: 14) |

Suppression Subtractive Hybridization:

Suppression Subtractive Hybridization (SSH) was used to identify cDNAs corresponding to genes that may be differentially expressed in prostate cancer. The SSH reaction utilized cDNA from two LAPC-4 AD (androgen-dependent) xenografts. Specifically, the 98P7C3 SSH sequence was identified from a subtraction where cDNA derived from LAPC-4 AD grown orthotopically in the prostate was subtracted from cDNA derived from an LAPC-4 AD tumor grown in the tibia. The SSH DNA sequence of 188 bp (FIG. 1) was identified.

The LAPC-4 AD xenograft grown in the tibia was used as the source of the “tester” cDNA, while the cDNA from the LAPC-4 AD xenograft grown in the prostate was used as the source of the “driver” cDNA. Double stranded cDNAs corresponding to tester and driver cDNAs were synthesized from 2 μg of poly(A)⁺ RNA isolated from the relevant xenograft tissue, as described above, using CLONTECH's PCR-Select cDNA Subtraction Kit and 1 ng of oligonucleotide DPNCDN as primer. First- and second-strand synthesis were carried out as described in the Kit's user manual protocol (CLONTECH Protocol No. PT1117-1, Catalog No. K1804-1). The resulting cDNA was digested with Dpn II for 3 hrs at 37° C. Digested cDNA was extracted with phenolchloroform (1:1) and ethanol precipitated.

Driver cDNA was generated by combining in a 1:1 ratio Dpn II digested cDNA from the relevant xenograft source (see above) with a mix of digested cDNAs derived from the human cell lines HeLa, 293, A431, Colo205, and mouse liver.

Tester cDNA was generated by diluting 1 μl of Dpn II digested cDNA from the relevant xenograft source (see above) (400 ng) in 5 μl of water. The diluted cDNA (2 μl, 160 ng) was then ligated to 2 μl of Adaptor 1 and Adaptor 2 (10 μM), in separate ligation reactions, in a total volume of 10 μl at 16° C. overnight, using 400 u of T4 DNA ligase (CLONTECH). Ligation was terminated with 1 μl of 0.2 M EDTA and heating at 72° C. for 5 min.

The first hybridization was performed by adding 1.5 μl (600 ng) of driver cDNA to each of two tubes containing 1.5 μl (20 ng) Adaptor 1- and Adaptor 2-ligated tester cDNA. In a final volume of 4 μl, the samples were overlaid with mineral oil, denatured in an M J Research thermal cycler at 98° C. for 1.5 minutes, and then were allowed to hybridize for 8 hrs at 68° C. The two hybridizations were then mixed together with an additional 1 μl of fresh denatured driver cDNA and were allowed to hybridize overnight at 68° C. The second hybridization was then diluted in 200 μl of 20 mM Hepes, pH 8.3, 50 mM NaCl, 0.2 mM EDTA, heated at 70° C. for 7 min. and stored at −20° C.

PCR Amplification, Cloning and Sequencing of Gene Fragments Generated from SSH:

To amplify gene fragments resulting from SSH reactions, two PCR amplifications were performed. In the primary PCR reaction 1 μl of the diluted final hybridization mix was added to 1 μl of PCR primer 1 (10 μM), 0.5 μl dNTP mix (10 μM), 2.5 μl 10×reaction buffer (CLONTECH) and 0.5 μl 50×Advantage cDNA polymerase Mix (CLONTECH) in a final volume of 25 μl. PCR 1 was conducted using the following conditions: 75° C. for 5 min., 94° C. for 25 sec., then 27 cycles of 94° C. for 10 sec, 66° C. for 30 sec, 72° C. for 1.5 min. Five separate primary PCR reactions were performed for each experiment. The products were pooled and diluted 1:10 with water. For the secondary PCR reaction, 1 μl from the pooled and diluted primary PCR reaction was added to the same reaction mix as used for PCR 1, except that primers NP 1 and NP2 (10 μM) were used instead of PCR primer 1. PCR 2 was performed using 10-12 cycles of 94° C. for 10 sec, 68° C. for 30 sec, and 72° C. for 1.5 minutes. The PCR products were analyzed using 2% agarose gel electrophoresis.

The PCR products were inserted into pCR2.1 using the T/A vector cloning kit (Invitrogen). Transformed E. coli were subjected to blue/white and ampicillin selection. White colonies were picked and arrayed into 96 well plates and were grown in liquid culture overnight. To identify inserts, PCR amplification was performed on 1 ml of bacterial culture using the conditions of PCR1 and NP1 and NP2 as primers. PCR products were analyzed using 2% agarose gel electrophoresis.

Bacterial clones were stored in 20% glycerol in a 96 well format. Plasmid DNA was prepared, sequenced, and subjected to nucleic acid homology searches of the GenBank, dBest, and NCI-CGAP databases.

RT-PCR Expression Analysis:

First strand cDNAs were generated from 1 μg of mRNA with oligo (dT)12-18 priming using the Gibco-BRL Superscript Preamplification system. The manufacturer's protocol was used which included an incubation for 50 min at 42° C. with reverse transcriptase followed by RNAse H treatment at 37° C. for 20 min. After completing the reaction, the volume was increased to 200 μl with water prior to normalization.

Normalization of the first strand cDNAs from multiple normal and cancer tissues was performed by using β-actin primers. First strand cDNA (5 μl) were amplified in a total volume of 50 μl containing 0.4 μM primers, 0.2 μM each dNTPs, 1×PCR buffer (Gibco-BRL, 10 mM Tris-HCL, 1.5 mM MgCl₂, 50 mM KCl, pH8.3) and 1×Platinum Taq DNA polymerase (Gibco-BRL). PCR was performed using an M J Research thermal cycler under the following conditions: Initial denaturation can be at 94° C. for 45 sec, followed by a 18, 20, and 22 cycles of 94° C. for 45, 58° C. for 45 sec, 72° C. for 45 sec. A final extension at 72° C. was carried out for 2 min. Five μl of the PCR reaction were removed at 18, 20, and 22 cycles and used for agarose gel electrophoresis. After agarose gel electrophoresis, the band intensities of the 283 b.p. β-actin bands from multiple tissues were compared by visual inspection. Dilution factors for the first strand cDNAs were calculated to result in equal β-actin band intensities in all tissues after 22 cycles of PCR. Three rounds of normalization can be required to achieve equal band intensities in all tissues after 22 cycles of PCR.

To determine expression levels of the gene, 5 μl of normalized first strand cDNA were analyzed by PCR using 26, and 30 cycles of amplification. Semi-quantitative expression analysis was achieved by comparing the PCR products at cycle numbers that give light band intensities. RT-PCR expression analysis was performed on first strand cDNAs generated using pools of tissues from multiple normal and cancer samples. The cDNA normalization was demonstrated in every experiment using beta-actin PCR.

### Example 2

### Full Length Cloning of 98P7C3 and Homology Comparison to Known Sequences

A full length 98P7C3 cDNA clone (clone EBB10) of 1129 base pairs (bp) was cloned from a prostate library (Lambda ZAP Express, Stratagene) (FIG. 2). The cDNA encodes a putative open reading frame (ORF) of 73 amino acids with calculated MW of 8.2 kDa, and PI of 4.8. 98P7C3 is predicted to be a nuclear protein (43%), with a possibility of cytoplasmic localization (3 0%).

98P7C3 shows best homology to an unnamed human protein, specifically gi|13560281, with 100% identity. Based on amino acid sequence, 98P7C3 demonstrates significant homology to PAX6, with 37% identity and 54% homology (FIG. 4) over the entire length of the 98P7C3 protein. Sequence analysis of 98P7C3 also reveals homology to the newt homeobox protein goosecoid. The two protein sequences are 43% identical and 60% homologous over a 57 amino acid region of 98P7C3 and a 58 amino acid region within the goosecoid homeobox domain (FIG. 5). Sequence analysis of 98P7C3 identifies a homeobox motif at amino acids 10-60. Proteins containing homeobox domains are primarily sequence-specific DNA-binding transcription factors that participate in the regulation of developmental processes.

PAX genes are similar to homeobox genes, except that for a homeobox motif the homeodomain carries about 60 amino acids while the equivalent element in PAX genes is about 128 amino acids. Several PAX genes also contain a homeobox (Strachan, T. and Read, A. Curr. Opin. Genetics Devel. 1994, 4:427). PAX genes are members of a family of transcription factors that play a role in development. PAX6 is mostly expressed in the central nervous system, specifically in the brain and ventral ventricular zone of the neural tube; in the nose, the developing eye and the pituitary. There is evidence to indicate that PAX6 plays a role in the development of the nervous system (Marquardt T. et al, Cell. 2001, 105:43; Callaerts P. J Neurobiol. 2001, 46:73). Recent evidence indicates that PAX6 is ectopically expressed in colon and bladder cancer (Salem C. et al, Int J Cancer 2000;87:179).

The 98P7C3 cDNA has been deposited under the requirements of the Budapest Treaty on Feb. 2, 2000, with the American Type Culture Collection (ATCC), 10801 University Blvd., Manassas, Va. 20110-2209 USA, as plasmid p98P7C3-EBB10, and has been assigned Accession No. PTA-1261.

### Example 3

### Chromosomal Mapping of the 98P7C3 Gene

The 98P7C3 gene was mapped to chromosome 4 using the Coriell chromosomal panel (Coriell Institute for Medical Research, Camden, N.J.). Bioinformatics analysis of the UniGene corresponding to 98P7C3 (identified using EST A1683579) indicates that this gene maps to chromosome 4q11-q12 (UniGene database in NCBI at http://www.ncbi.nlm.nih.gov/UniGene/Hs.Home.html).

### Example 4

### Expression Analysis of 98P7C3 in Normal Tissues, Cancer Cell Lines and Patient Samples

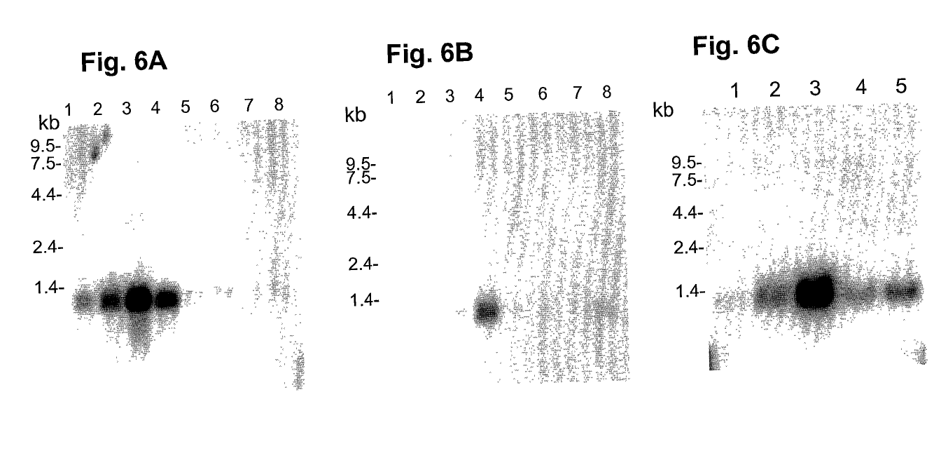

98P7C3 mRNA expression in normal human tissues was analyzed by Northern blotting of two multiple tissue blots (Clontech; Palo Alto, Calif.), comprising a total of 16 different normal human tissues, using labeled 98P7C3 SSH fragment (Example 1) as a probe. RNA samples were quantitatively normalized with a β-actin probe. The results demonstrated expression of a 1.2 kb transcript in placenta, lung, brain, testis and heart (FIGS. 6A-C).

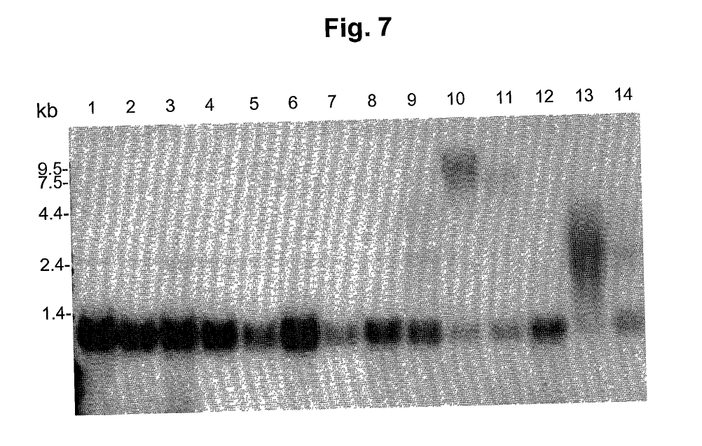

To analyze 98P7C3 expression in prostate cancer tissues, northern blotting was performed on RNA derived from the LAPC xenografts. The results showed expression in all the xenografts, with higher levels detected in the androgen-independent tissues, especially LAPC-4 AI. More detailed analysis of the xenografts shows that 98P7C3 is highly expressed in the xenografts even when grown within the tibia of mice (LAPC-4 AD it and LAPC-9 AD it) (FIG. 7). Similarly, high expression was also detected in a xenograft that was grown within human bone explants in SCID mice (LAPC-4 AD2). This indicates that bone growth of these prostate cancer tissues does not diminish their expression.

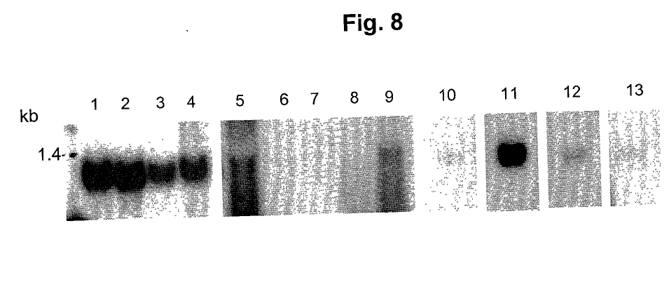

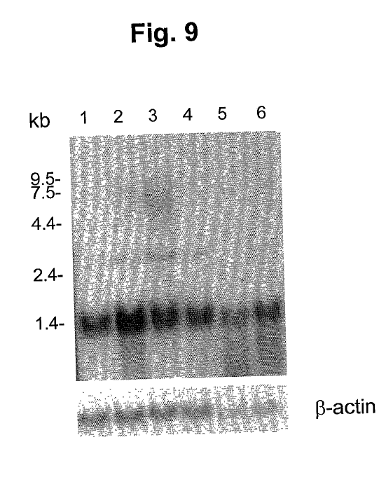

To analyze 98P7C3 expression in various cancer tissues, northern blotting was performed on RNA derived from several prostate and non-prostate cancer cell lines. The results show that, in addition to the LAPC-xenografts, expression was detected in several cancer cell lines derived from prostate (LNCaP, LAPC-4 CL), pancreas (HPAC), leukemia (KCL22), bone (HOS), and breast (CAMA-1) (FIG. 8). Northern analysis also shows that 98P7C3 is expressed in the normal prostate and prostate tumor tissues derived from prostate cancer patients (FIG. 9). The detection of 98P7C3 expression in the normal adjacent tissue of the prostate cancer patients may be due to the presence of cancer glands within the normal tissue. Alternatively, an up-regulation of 98P7C3 may manifest itself early in the disease, even in the seemingly normal part of the prostate.

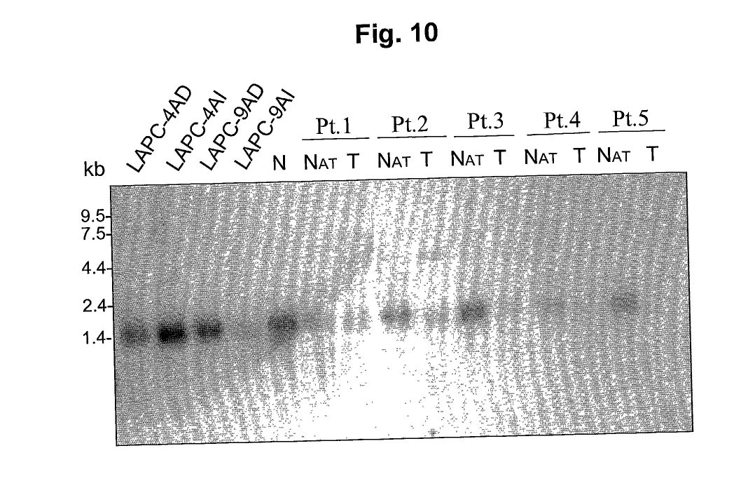

FIG. 10 shows expression of 98P7C3 in prostate cancer patient samples. RNA was extracted from prostate cancer xenografts (LAPC-4AD, LAPC-4AI, LAPC-9AD and LAPC-9AI), normal prostate (N), prostate tumors (T) and their normal adjacent tissues (NAT) derived from prostate cancer patients. Northern blots with 10 μg of total RNA/lane were probed with the 98P7C3 sequences. Size standards in kilobases (kb) are indicated on the left side of the Figure. Results show expression of 98P7C3 in all patients and in all four xenografts.

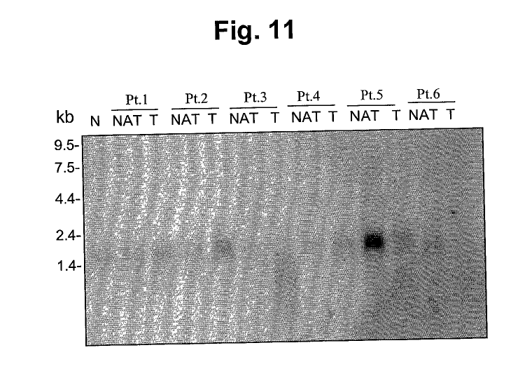

FIG. 11 shows expression of 98P7C3 in kidney cancer patient samples. RNA was extracted from normal kidney (N), kidney tumors (T) and their normal adjacent tissues (NAT), the tumor tissues derived from kidney cancer patients. Northern blots with 10 μg of total RNA/lane were probed with the 98P7C3 sequences. Size standards in kilobases (kb) are indicated on the left side of the Figure. Results show expression of 98P7C3 in all kidney specimens tested.

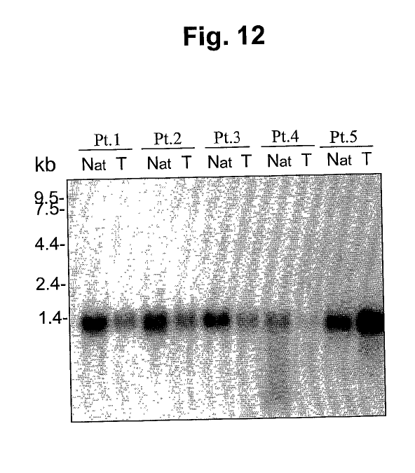

FIG. 12 shows expression of 98P7C3 in lung cancer patient samples. RNA was extracted from lung tumors (T) and their normal adjacent tissues (NAT), the tumor tissues derived from lung cancer patients. Northern blots with 10 μg of total RNA/lane were probed with the 98P7C3 sequences. Size standards in kilobases (kb) are indicated on the left side of the Figure. Results show expression of 98P7C3 in all lung specimens tested.

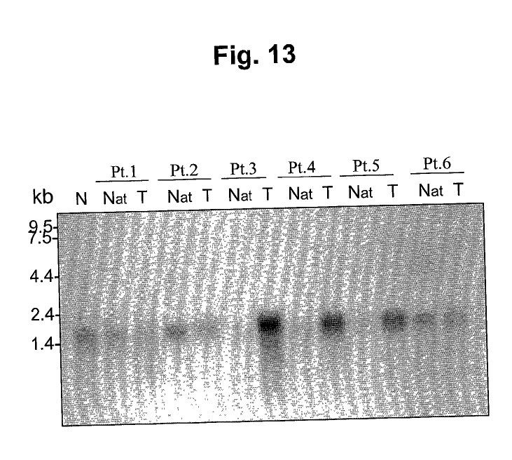

FIG. 13 shows expression of 98P7C3 in colon cancer patient samples. RNA was extracted from normal colon (N), colon tumors (T) and their normal adjacent tissues (NAT), the tumor tissues derived from colon cancer patients. Northern blots with 10 μg of total RNA/lane were probed with the 98P7C3 sequences. Size standards in kilobases (kb) are indicated on the left side of the Figure. Results show expression of 98P7C3 in all colon specimens tested. Strong upregulation in tumor expression compared to normal adjacent tissues is seen in 3 out of 6 patients.

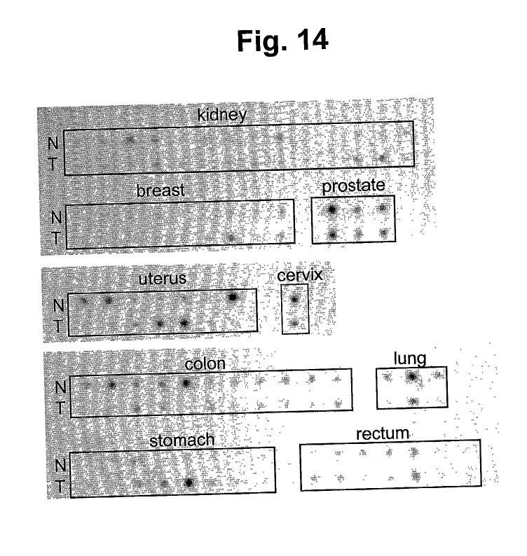

FIG. 14 shows expression of 98P7C3 in human patient cancer specimens and cancer cell lines. Expression of 98P7C3 was assayed in a panel of human cancers (T) and their respective matched normal tissues (N) on RNA dot blots. 98P7C3 expression was seen in kidney, breast, prostate, uterus, cervix, colon, lung, stomach and rectum cancers. The expression detected in some normal adjacent tissues (isolated from diseased tissues), but not in normal tissues (isolated from healthy donors), may indicate that these tissues are not fully normal and that 98P7C3 may be expressed in early stage tumors.

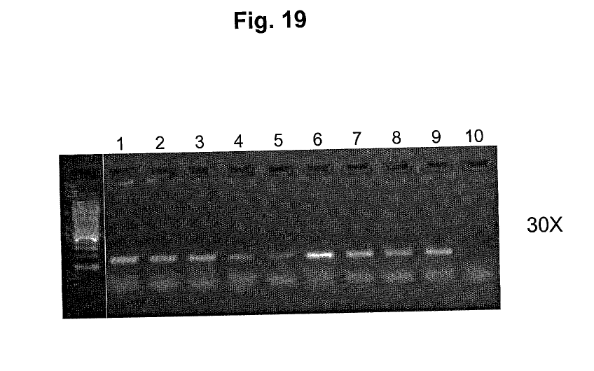

FIG. 19 shows the results of RT-PCR analysis of 98P7C3 expression in various pooled cancer tissues after 30 cycles. 98P7C3 expression was observed in each of the following tissues: xenograft pool (lane 1), prostate cancer pool (lane 2), bladder cancer pool (lane 3), kidney cancer pool (lane 4), colon cancer pool (lane 5), lung cancer pool (lane 6), ovarian cancer pool (lane 7), breast cancer pool (lane 8), metastasis pool (lane 9). Lane 10 is a water blank.

These results suggest that 98P7C3 is generally up-regulated in cancer cells and cancer tissues, especially from prostate cancer, and provides a suitable target for cancer therapy.

### Example 5

### Generation of 98P7C3 Polyclonal Antibodies

Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. In addition to immunizing with the full length 98P7C3 protein, computer algorithms are employed in design of immunogens that based on amino acid sequence analysis contain characteristics of being antigenic and available for recognition by the immune system of the immunized host (see below, Example entitled “Amino Acid Scale Profiles and Identification of Antigenic Regions of 98P7C3”). Such regions would be predicted to be hydrophilic, flexible, in beta-turn conformations, and be exposed on the surface of the protein (see, e.g., FIG. 15, FIG. 17, FIG. 17, or FIG. 18 for amino acid profiles that predict such regions of 98P7C3).

For example, 98P7C3 recombinant bacterial fusion proteins or peptides encoding hydrophilic, flexible, beta-turn regions of the 98P7C3 sequence, such as the amino terminal portion of 98P7C3 are used to immunize New Zealand White rabbits. The peptide can be conjugated to keyhole limpet hemocyanin (KLH) and used to immunize the rabbit. Alternatively the immunizing agent may include all or portions of the 98P7C3 protein, analogs or fusion proteins thereof. For example, the 98P7C3 amino acid sequence can be fused to any one of a variety of fusion protein partners that are well known in the art, such as maltose binding protein, LacZ, thioredoxin or an immunoglobulin constant region (see e.g. Current Protocols In Molecular Biology, Volume 2, Unit 16, Frederick M. Ausubul et al. eds., 1995; Linsley, P. S., Brady, W., Umes, M., Grosmaire, L., Damle, N., and Ledbetter, L. (1991) J.Exp. Med. 174, 561-566). Other recombinant bacterial proteins include glutathione-S-transferase (GST), and HIS tagged fusion proteins of 98P7C3 that are purified from induced bacteria using the appropriate affinity matrix.

It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate).

In a typical protocol, rabbits are initially immunized subcutaneously with up to 200 μg, typically 100-200 μg, of fusion protein or peptide conjugated to KLH mixed in complete Freund's adjuvant. Rabbits are then injected subcutaneously every two weeks with up to 200 pig, typically 100-200 μg, of immunogen in incomplete Freund's adjuvant. Test bleeds are taken approximately 7-10 days following each immunization and used to monitor the titer of the antiserum by ELISA.

To test serum, such as rabbit serum, for reactivity with 98P7C3 proteins, the full-length 98P7C3 cDNA can be cloned into an expression vector such as one that provides a 6His tag at the carboxyl-terminus (pCDNA 3.1 myc-his, Invitrogen). After transfection of the constructs into 293T cells, cell lysates can be probed with anti-His antibody (Santa Cruz Biotechnologies, Santa Cruz, Calif.) and the anti-98P7C3 serum using Western blotting. Alternatively specificity of the antiserum is tested by Western blot and immunoprecipitation analyses using lysates of cells that express 98P7C3. Serum from rabbits immunized with GST or MBP fusion proteins is first semi-purified by removal of anti-GST or anti-MBP antibodies by passage over GST and MBP protein columns respectively. Sera from His-tagged protein and peptide immunized rabbits as well as depleted GST and MBP protein sera are purified by passage over an affinity column composed of the respective immunogen covalently coupled to Affigel matrix (BioRad).

### Example 6

### Generation of 98P7C3 Monoclonal Antibodies (mAbs)

In one embodiment, therapeutic mAbs to 98P7C3 will include those that react with epitopes of the protein that would disrupt or modulate the biological function of 98P7C3. Immunogens for generation of such mabs are designed to encode or contain the entire 98P7C3 protein or regions of the 98P7C3 protein. In addition to immunizing with the full length 98P7C3 protein, computer algorithms are employed in design of immunogens that based on amino acid sequence analysis contain characteristics of being antigenic and available for recognition by the immune system of the immunized host (see below, Example entitled “Amino Acid Scale Profiles and Identification of Antigenic Regions of 98P7C3”). Such regions would be predicted to be hydrophilic, flexible, in beta-turn conformations, and be exposed on the surface of the protein (see, e.g., FIG. 15, FIG. 17, FIG. 17, or FIG. 18 for amino acid profiles that predict such regions of 98P7C3). Immunogens include peptides, recombinant bacterial proteins, and mammalian expressed Tag5 proteins and human and murine IgG FC fusion proteins.

To generate mAbs to 98P7C3, mice are first immunized intraperitoneally (IP) with typically 10-50 μg, of protein immunogen mixed in complete Freund's adjuvant. Mice are then subsequently immunized IP every 2-4 weeks with typically 10-50 μg, of antigen mixed in Freund's incomplete adjuvant. Alternatively, Ribi adjuvant is used immunizations. In addition, a DNA-based immunization protocol is employed in which a mammalian expression vector encoding 98P7C3 sequence is used to immunize mice by direct injection of the plasmid DNA. For example, pCDNA 3.1 encoding either the full length 98P7C3 cDNA or the amino-terminal region of 98P7C3 predicted to be antigenic from sequence analysis (see, e.g., Figure W, Figure X, Figure Y, or Figure Z) fused to the coding sequence of murine or human IgG are used. This protocol is used alone or in combination with protein immunogens. Test bleeds are taken 7-10 days following immunization to monitor titer and specificity of the immune response. Once appropriate reactivity and specificity is obtained as determined by ELISA, Western blotting, and immunoprecipitation analyses, fusion and hybridoma generation is then carried with established procedures well known in the art (Harlow and Lane, 1988).

In one embodiment for generating 98P7C3 monoclonal antibodies, a glutathione-S-transferase (GST) fusion protein encoding the full length 98P7C3 protein is expressed, purified, and used as immunogen. Balb C mice are initially immunized intraperitoneally with 25 μg of the GST-98P7C3 fusion protein mixed in complete Freund's adjuvant. Mice are subsequently immunized every two weeks with 25 μg of GST-98P7C3 protein mixed in Freund's incomplete adjuvant for a total of three immunizations. To determine titer of serum from immunized mice, ELISA is carried out using a 98P7C3-specific cleavage fragment of the immunogen in which GST is removed by site specific proteolysis. Reactivity and specificity of serum to fall length 98P7C3 protein is monitored by Western blotting and flow cytometry using 293T cells transfected with an expression vector encoding the 98P7C3 cDNA (Example 7). Mice showing the strongest reactivity are rested for three weeks and given a final injection of 98P7C3 cleavage fragment in PBS and then sacrificed four days later. The spleens of the sacrificed mice are then harvested and fused to SPO/2 myeloma cells using standard procedures (Harlow and Lane, 1988). Supernatants from growth wells following HAT selection are screened by ELISA, Western blot, and flow cytometry to identify 98P7C3 specific antibody-producing clones.

The binding affinity of a 98P7C3 monoclonal antibody is determined using standard technologies. Affinity measurements quantify the strength of antibody to epitope binding and can be used to help define which 98P7C3 monoclonal antibodies are preferred for diagnostic or therapeutic use. The BIAcore system (Uppsala, Sweden) is a preferred method for determining binding affinity. The BIAcore system uses surface plasmon resonance (SPR, Welford K. 1991, Opt. Quant. Elect. 23:1; Morton and Myszka, 1998, Methods in Enzymology 295: 268) to monitor biomolecular interactions in real time. BIAcore analysis conveniently generates association rate constants, dissociation rate constants, equilibrium dissociation constants, and affinity constants.

### Example 7

### Production of Recombinant 98P7C3 in Bacterial and Mammalian Systems

BACTERIAL CONSTRUCTS

pGEX Constructs

To express 98P7C3 in bacterial cells, cDNA coding for the 73 amino acid ORF (such as that encoding amino acids 1 to 73 or any 8, 9, 10, 11, 12, 13, 14, or 15 contiguous amino acids from 98P7C3 or an analog thereof) is fused to the Glutathione S-transferase (GST) gene by cloning into pGEX-6P-1 (Amersham Pharmacia Biotech, NJ). The constructs are made in order to generate recombinant 98P7C3 protein sequences with GST fused at the N-terminus and a six histidine epitope at the C-terminus. The six histidine epitope tag is generated by adding the histidine codons to the cloning primer at the 3′ end of the open reading frame (ORF). A PreScission™ recognition site permits cleavage of the GST tag from 98P7C3-related protein. The ampicillin resistance gene and pBR322 origin permits selection and maintenance of the plasmid in E. coli.

pMAL Constructs

To express 98P7C3 in bacterial cells, all or part of the 98P7C3 nucleic acid sequence (such as that encoding amino acids 1 to 73 or any 8, 9, 10, 11, 12, 13, 14, or 15 contiguous amino acids from 98P7C3 or an analog thereof) are fused to the maltose-binding protein (MBP) gene by cloning into pMAL-c2X and pMAL-p2X (New England Biolabs, Mass.). The constructs are made to generate recombinant 98P7C3 protein sequences with MBP fused at the N-terminus and a six histidine epitope at the C-termiinus. The six histidine epitope tag is generated by adding histidine codons to the 3′ cloning primer. A Factor Xa recognition site permits cleavage of the GST tag from 98P7C3. The pMAL-c2X and pMAL-p2X vectors are optimized to express the recombinant protein in the cytoplasm or periplasm respectively. Periplasm expression enhances folding of proteins with disulfide bonds.

pCRII

To generate 98P7C3 sense and anti-sense riboprobes for RNA in situ investigations, a pCRII construct (Invitrogen, Carlsbad, Calif.) is generated using cDNA sequence encoding the ORF or fragments of the cDNA (such as that encoding amino acids 1 to 73 or any 8, 9, 10, 11, 12, 13, 14, or 15 contiguous amino acids from 98P7C3 or an analog thereof). The pCRII vector has Sp6 and T7 promoters flanking the insert to drive the production of 98P7C3 RNA riboprobes for use in RNA in situ hybridization experiments.

MAMMALIAN CONSTRUCTS

To express recombinant 98P7C3, the full or partial length 98P7C3 cDNA (such as that encoding amino acids I to 73 or any 8, 9, 10, 11, 12, 13, 14, or 15 contiguous amino acids from 98P7C3 or an analog thereof) can be cloned into any one of a variety of expression vectors known in the art. The constructs can be transfected into any one of a wide variety of mammalian cells such as 293T cells. Transfected 293T cell lysates can be probed with anti-98P7C3 polyclonal serum in a Western blot experiment.

The 98P7C3 gene and cDNA fragments can also be subcloned into the retroviral expression vector pSRαMSVtkneo and used to establish 98P7C3-expessing cell lines as follows: The 98P7C3 coding sequence (from translation initiation ATG and Kozak translation start consensus sequence to the termination codons) is amplified by PCR using the 98P7C3 cDNA. The PCR product is subcloned into pSRαMSVtkneo vector and transformed into DH5α competent cells. Colonies are picked to screen for clones with unique internal restriction sites on the cDNA. The positive clone is confirmed by sequencing of the cDNA insert. The retroviral vectors can thereafter be used for infection and generation of various cell lines using, for example, NIH 3T3, TsuPr1, 293 or rat-1 cells.

Additional illustrative mammalian systems are discussed below.

pcDNA4/HisMax-TOPO Constructs

To express 98P7C3 in mammalian cells, the 98P7C3 ORF (such as that encoding amino acids 1 to 73 or any 8, 9, 10, 11, 12, 13, 14, or 15 contiguous amino acids from 98P7C3 or an analog thereof) is cloned into pcDNA4/HisMax-TOPO Version A (cat# K864-20, Invitrogen, Carlsbad, Calif.). Protein expression is driven from the cytomegalovirus (CMV) promoter and the SP 163 translational enhancer. The recombinant protein has Xpress™ and six histidine epitopes fused to the N-terminus. The pcDNA4/HisMax-TOPO vector also contains the bovine growth hormone (BGH) polyadenylation signal and transcription termination sequence to enhance mRNA stability along with the SV40 origin for episomal replication and simple vector rescue in cell lines expressing the large T antigen. The Zeocin resistance gene allows for selection of mammalian cells expressing the protein and the ampicillin resistance gene and ColE1 origin permits selection and maintenance of the plasmid in E. coli.

pcDNA3.1/MycHis Constructs

To express 98P7C3 in mammalian cells, the ORF (such as that encoding amino acids 1 to 73 or any 8, 9, 10, 11, 12, 13, 14, or 15 contiguous amino acids from 98P7C3 or an analog thereof) with consensus Kozak translation initiation site is cloned into pcDNA3.1/MycHis_Version A (Invitrogen, Carlsbad, Calif.). Protein expression is driven from the cytomegalovirus (CMV) promoter. The recombinant protein has the myc epitope and six histidines fused to the C-terminus. The pcDNA3.1/MycHis vector also contains the bovine growth hormone (BGH) polyadenylation signal and transcription termination sequence to enhance mRNA stability, along with the SV40 origin for episomal replication and simple vector rescue in cell lines expressing the large T antigen. The Neomycin resistance gene can be used, as it allows for selection of mammalian cells expressing the protein and the ampicillin resistance gene and ColE1 origin permits selection and maintenance of the plasmnidin E. coli.

pcDNA3.1/Vhis-TOPO Construct

To express 98P7C3 in mammalian cells, the cDNA encoding the 98P7C3 ORF, or amino acids 1 to 73 or any 8, 9, 10, 11, 12, 13, 14, or 15 contiguous amino acids from 98P7C3 or an analog thereof along with Kozak consensus translation initiation sequence are clone into pcDNA4/V5His-TOPO (cat# K4800-01, Invitrogen, Carlsbad, Calif.). Protein expression is driven form the cytomegalovirus (CMV) promoter. The recombinant protein has V₅™ and six histidine epitopes fused at the C-terminus to aid in detection and purification of the recombinant protein. The pcDNA4/V5His-TOPO vector also contains the bovine growth hormone (BGH) polyadenylation signal and transcription termination sequence to enhance mRNA stability along with the SV40 origin for episomal replication and simple vector rescue in cell lines expressing the large T antigen. The Zeocin resistance gene allows for selection of mammalian cells expressing the protein and the ampicillin resistance gene and the ColE1 origin permits selection and maintenance of the plasmid in E. coli.

pcDNA3.ICT-GFP-TOPO Construct

To express 98P7C3 in mammalian cells and to allow detection of the recombinant protein using fluorescence, the cDNA coding for the ORF (such as that encoding amino acids 1 to 73 or any 8, 9, 10, 11, 12, 13, 14, or 15 contiguous amino acids from 98P7C3 or an analog thereof) with consensus Kazoo translation initiation site is cloned into pcDNA3.1CT-GFP-TOPO (Invitrogen, Calif.). Protein expression is driven from the cytomegalovirus (CMV) promoter. The recombinant protein has the Green Fluorescent Protein (GFP) fused to the C-terminus facilitating non-invasive, in vivo detection and cell biology studies. The pcDNA3.1/MycHis vector also contains the bovine growth hormone (BGH) polyadenylation signal and transcription termination sequence to enhance mRNA stability along with the SV40 origin for episomal replication and simple vector rescue in cell lines expressing the large T antigen. The Neomycin resistance gene allows for selection of mammalian cells that express the protein, and the ampicillin resistance gene and ColE1 origin permits selection and maintenance of the plasmid in E. coli. An additional construct with a N-terminal GFP fusion is made in pcDNA3.1NT-GFP-TOPO spanning the entire length of the 98P7C3 protein.

pAPtag

The cDNA coding for the 98P7C3 ORF (such as that encoding amino acids 1 to 73 or any 8, 9, 10, 11, 12, 13, 14, or 15 contiguous amino acids from 98P7C3 or an analog thereof) is cloned into pAPtag-5 (GenHunter Corp. Nashville, Tenn.). This construct generates an alkaline phosphatase fusion at the C-terminus of the 98P7C3 protein while fusing the IgGK signal sequence to N-terminus. The resulting recombinant 98P7C3 protein is optimized for secretion into the media of transfected mammalian cells and can be used to identify proteins such as ligands or receptors that interact with the 98P7C3 protein. Protein expression is driven from the CMV promoter and the recombinant protein also contains myc and six histidines fused to the C-terminus of alkaline phosphatase. The Zeosin resistance gene allows for selection of mammalian cells expressing the protein and the ampicillin resistance gene permits selection of the plasmid in E. coli.

ptag5

The cDNA coding for the 98P7C3 ORF (such as that encoding amino acids 1 to 73 or any 8, 9, 10, 11, 12, 13, 14, or 15 contiguous amino acids from 98P7C3 or an analog thereof) is also cloned into pTag-5. This vector is similar to pAPtag but without the alkaline phosphatase fusion. This construct fuses the IgGK signal sequence to the N-terminus of inserts. The resulting recombinant 98P7C3 protein is optimized for secretion into the media of transfected mammalian cells, and can be used to identify proteins such as ligands or receptors that interact with the 98P7C3 protein. Protein expression is driven from the CMV promoter and the recombinant protein also contains myc and six histidines fused to the C-terminus of alkaline phosphatase. The Zeosin resistance gene allows for selection of mammalian cells expressing the protein, and the ampicillin resistance gene permits selection of the plasmid in E. coli.

psecFc

The cDNA coding for the 98P7C3 ORF (such as that encoding amino acids 1 to 73 or any 8, 9, 10, 11, 12, 13, 14, or 15 contiguous amino acids from 98P7C3 or an analog thereof) is cloned into psecFc. The psecFc vector was assembled by cloning immunoglobulin G1 Fc (hinge, CH2, CH3 regions) into pSecTag2 (Invitrogen, Calif.). This construct generates an immunoglobulin G1 Fc fusion at the C-terminus of the 98P7C3 protein, while fusing the IgGK signal sequence to N-terminus. The resulting recombinant 98P7C3 protein is optimized for secretion into the media of transfected mammalian cells, and can be used to identify proteins such as ligands or receptors that interact with the 98P7C3 protein. Protein expression is driven from the CMV promoter and the recombinant protein also contains myc and six histidines fused to the C-terminus of alkaline phosphatase. The Zeosin resistance gene allows for selection of mammalian cells that express the protein, and the ampicillin resistance gene permits selection of the plasmid in E. coli.

pSRα Constructs

To generate mammalian cell lines that express 98P7C3 constitutively, the cDNA coding for the ORF (such as that encoding amino acids 1 to 73 or any 8, 9, 10, 11, 12, 13, 14, or 15 contiguous amino acids from 98P7C3 or an analog thereof) is cloned into pSRα constructs. Amphotropic and ecotropic retroviruses were generated by transfection of pSRα constructs into the 293T-10A1 packaging line or co-transfection of pSRα and a helper plasmid (\tilde{φ}) in the 293 cells, respectively. The retrovirus can be used to infect a variety of mammalian cell lines, resulting in the integration of the cloned gene, 98P7C3, into the host cell-lines. Protein expression is driven from a long terminal repeat (LTR). The Neomycin resistance gene allows for selection of mammalian cells that express the protein, and the ampicillin resistance gene and ColE1 origin permit selection and maintenance of the plasmid in E. coli.

An additional pSRα construct was made that fused the FLAG tag to the C-terminus to allow detection using anti-FLAG antibodies. The FLAG sequence 5′ gat tac aag gat gac gac gat aag 3′ was added to the cloning primer at the 3′ end of the ORF.

Additional pSRα constructs are made to produce both N-terminal and C-terminal GFP and myc/6 HIS fusion proteins of the full-length 98P7C3 protein.

### Example 8

### Production of Recombinant 98P7C3 in a Baculovirus System

To generate a recombinant 98P7C3 protein in a baculovirus expression system, cDNA sequence encoding the 98P7C3 protein is cloned into the baculovirus transfer vector pBlueBac 4.5 (Invitrogen), which provides a His-tag at the N-terminus Specifically, pBlueBac—98P7C3 is co-transfected with helper plasmid pBac-N-Blue (Invitrogen) into SF9 (Spodoptera frugiperda) insect cells to generate recombinant baculovirus (see Invitrogen instruction manual for details). Baculovirus is then collected from cell supernatant and purified by plaque assay.

Recombinant 98P7C3 protein is then generated by infection of HighFive insect cells (Invitrogen) with the purified baculovirus. Recombinant 98P7C3 protein can be detected using anti-98P7C3 antibody. 98P7C3 protein can be purified and used in various cell-based assays or as immunogen to generate polyclonal and monoclonal antibodies specific for 98P7C3.

### Example 9

### Identification of Potential Signal Transduction Pathways

Many mammalian proteins have been reported to interact with signaling molecules and to participate in regulating signaling pathways (J Neurochem. 2001; 76:217-223). Homeobox containing proteins can interact with other molecules through a helix-loop-helix motif, as well as other motifs associated with protein-protein interactions. Using immunoprecipitation and Western blotting techniques, we identify proteins that associate 98P7C3 and mediate signaling events. Several pathways known to play a role in cancer biology can be regulated by several of these genes, including phospholipid pathways such as PI3K, AKT, etc, adhesion and migration pathways, including FAK, Rho, Rac-1, etc, as well as mitogenic/survival cascades such as ERK, p38, etc (Cell Growth Differ. 2000,11:279; J Biol Chem. 1999, 274:801; Oncogene. 2000, 19:3003, J. Cell Biol. 1997, 138:913.). Using Western blotting techniques, we can examine the ability of 98P7C3 to regulate of these pathways.

Cells expressing or lacking 98P7C3 are either left untreated or stimulated with cytokines, androgen and anti-integrin antibodies. Cell lysates are analyzed using anti-phosphos-specific antibodies (Cell Signaling, Santa Cruz Biotechnology) in order to detect phosphorylation and regulation of ERK, p38, AKT, PI3K, PLC and other signaling molecules. When 98P7C3 plays a role in the regulation of signaling pathways, whether individually or communally, it is used as a target for diagnostic, preventative and therapeutic purposes.

To determine whether 98P7C3 directly or indirectly activates known signal transduction pathways in cells, luciferase (luc) based transcriptional reporter assays are carried out in cells expressing individual genes. These transcriptional reporters contain consensus-binding sites for known transcription factors that lie downstream of well-characterized signal transduction pathways. The reporters and examples of these associated transcription factors, signal transduction pathways, and activation stimuli are listed below.

NFkB-luc, NFkB/Rel; Ik-kinase/SAPK; growth/apoptosis/stress

SRE-luc, SRF/TCF/ELK1; MA?K/SAPK; growth/differentiation

AP-1-luc, FOS/JUN; MAPK/SAPK/PKC; growth/apoptosis/stress

ARE-luc, androgen receptor; steroids/MAPK; growth/differentiation/apoptosis

p53-luc, p53; SAPK; growth/differentiation/apoptosis

CRE-luc, CREB/ATF2; PKA/p38; growth/apoptosis/stress

Gene-mediated effects can be assayed in cells showing mRNA expression. Luciferase reporter plasmids can be introduced by lipid-mediated transfection (TFX-50, Promega). Luciferase activity, an indicator of relative transcriptional activity, is measured by incubation of cell extracts with luciferin substrate and luminescence of the reaction is monitored in a luminometer.

Signaling pathways activated by 98P7C3 are mapped and used for the identification and validation of relevant targets. When these genes are involved in cell signaling, they are used as targets for diagnostic, prognostic, preventative and therapeutic purposes.

### Example 10

### Involvement in Tumor Progression

The 98P7C3 gene can contribute to the growth of cancer cells. The role of 98P7C3 in tumor growth is investigated in a variety of primary and transfected cell lines including prostate, colon, bladder and kidney cell lines as well as NIH 3T3 cells engineered to stably express one or more of these genes. Parental cells lacking 98P7C3 and cells expressing 98P7C3 are evaluated for cell growth using a well-documented proliferation assay (Fraser S P, Grimes J A, Djamgoz M B. Prostate 2000;44:61, Johnson D E, Ochieng J, Evans S L. Anticancer Drugs 1996, 7:288).

To determine the role of 98P7C3 the transformation process, we investigate its effect in colony forming assays. Parental NIH3T3 cells lacking 98P7C3 are compared to NHI-3T3 cells expressing 98P7C3, using a soft agar assay under stringent and more permissive conditions (Song Z. et al. Cancer Res. 2000 60:6730).

To determine the role of 98P7C3 in invasion and metastasis of cancer cells, we use a well-established Transwell Insert System assay (Becton Dickinson) (Cancer Res. 1999; 59:6010). Control cells, including prostate, colon, bladder and kidney cell lines lacking 98P7C3 are compared to cells expressing 98P7C3. Cells are loaded with the fluorescent dye, calcein, and plated in the top well of the Transwell insert coated with a basement membrane analog. Invasion is determined by fluorescence of cells in the lower chamber relative to the fluorescence of the entire cell population.

98P7C3 can also play a role in cell cycle and apoptosis. Parental cells and cells expressing 98P7C3 are compared for differences in cell cycle regulation using a well-established BrdU assay (Abdel-Malek Z A. J Cell Physiol. 1988, 136:247). In short, cells grown under both optimal (full serum) and limiting (low serum) conditions are labeled with BrdU and stained with anti-BrdU Ab and propidium iodide. Cells are analyzed for entry into the G1, S, and G2M phases of the cell cycle. Alternatively, the effect of stress on apoptosis is evaluated in control parental cells and cells expressing genes under consideration, including normal and tumor prostate, colon and lung cells. Engineered and parental cells are treated with various chemotherapeutic agents, such as etoposide, flutamide, etc, and protein synthesis inhibitors, such as cycloheximide. Cells are stained with annexin V-FITC and cell death is measured by FACS analysis. The modulation of cell death by 98P7C3 can play a critical role in regulating tumor progression and tumor load. When 98P7C3 plays a role in cell growth, transformation, invasion or apoptosis, it is used as a target for diagnostic, prognostic, preventative and therapeutic purposes.

### Example 11

### Involvement in Angiogenesis

Angiogenesis or new capillary blood vessel formation is necessary for tumor growth (Hanahan D, Folkman J. Cell. 1996, 86:353; Folkman J. Endocrinology. 1998 139:441). Several assays have been developed to measure angiogenesis in vitro and in vivo, for example tissue culture assays that evaluate endothelial cell tube formation and endothelial cell proliferation. Using these assays as well as in vitro neo-vascularization, one determines whether 98P7C3 enhances or inhibits angiogenesis.

For example, endothelial cells and cell lines are plated on an artificial basement membrane, such as matrigel, in the presence and absence of 98P7C3. The effect on tube formation is evaluated using light microscopy. In another embodiment, endothelial cells engineered to express 98P7C3 are evaluated using tube formation and proliferation assays. The effect of 98P7C3 is also evaluated in animal models in vivo. For example, cells either expressing or lacking 98P7C3 are implanted subcutaneously in immunocompromised mice. Endothelial cell migration and angiogenesis are evaluated 5-15 days later using immunohistochemistry techniques. When 98P7C3 affects angiogenesis, it is used as a target for diagnostic, prognostic, preventative and therapeutic purposes.

### Example 12

### Regulation of Transcription

The presence of a homeobox domain and the similarity to PAX genes indicate that 98P7C3 can play a role in transcriptional regulation of eukaryotic genes. Regulation of gene expression can be evaluated by studying gene expression in cells expressing or lacking 98P7C3. For this purpose, two types of experiments are performed.

In the first set of experiments, RNA from parental and 98P7C3-expressing cells are extracted and hybridized to commercially available gene arrays (Clontech) (Smid-Koopman E et al. Br J Cancer. 2000. 83:246). Resting cells as well as cells treated with FBS or androgen are compared. Differentially expressed genes are identified in accordance with procedures known in the art. The differentially expressed genes are then mapped to biological pathways (Chen K et al. Thyroid. 2001. 11:41.).

In the second set of experiments, specific transcriptional pathway activation is evaluated using commercially available (Stratagene) luciferase reporter constructs including: NFkB-luc, SRE-luc, ELK1-luc, ARE-luc, p53-luc, and CRE-luc. These transcriptional reporters contain consensus binding sites for known transcription factors that lie downstream of well-characterized signal transduction pathways, and represent a good tool to ascertain pathway activation and screen for positive and negative modulators of pathway activation.

When 98P7C3 plays a role in gene regulation, it is used as a target for diagnostic, prognostic, preventative and therapeutic purposes.

### Example 13

### Identification of 98P7C3-Specific Response Element

Homeobox proteins bind to DNA in a sequence specific manner, thereby regulating gene expression in genes carrying the homeobox recognition sequence (Norris P et al, DNA Cell Biol. 2001, 20:89; Sur IP and Toftgard R. .Mol Cell Biol Res Commun. 2000, 3:367). Using electrophoretic mobility shift assays (EMSA) and DNA footprinting, one can identify 98P7C3-binding DNA sequences. In short, nuclear lysates are extracted from parental 98P7C3-negative as well as 98P7C3-expressing cells. The lysates are incubated in the presence of 32-P-labeled DNA probes. DNA-protein complexes are either separated by electrophoresis or exposed to a restriction nuclease, and analyzed by radiography. This process provides 98P7C3-specific DNA element(s) that is a valuable tool in designing and testing inhibitors of 98P7C3. When 98P7C3 functions as a transcription factor, it is used as a target for diagnostic, prognostic, preventative and therapeutic purposes.

### Example 14

### Regulation of 98P7C3 Expression

The expression of homeobox containing protein can be regulated by several factors, including hormones and growth factors (Lin B et al, Cancer Res. 2000, 60:858; Kumar J P and Moses K, Cell 2001, 104:687). Using xenograft cells as well as other 98P7C3-expressing cells, one can study the regulation of 98P7C3 expression by hormones, such as androgen, estrogen, glucocorticoids and progesterone. Similarly, the effect of growth factors, secreted proteins and small molecules on 98P7C3 expression can be investigated in cells treated with EGF, FGF, VEGF, TGF, IGF and other proteins. These studies provide one of ordinary skill with means of regulating the expression of 98P7C3 and are valuable in the identification and preparation of diagnostic, prognostic, preventative and therapeutic compositions and methods. When the expression of 98P7C3 is modified molecules, it is used as a target for diagnostic, prognostic, preventative and therapeutic purposes.

### Example 15

### Protein-Protein Association

Several homeobox-containing proteins as well as PAX genes have been shown to interact with other proteins, thereby forming a protein complex that can regulate cell division, gene transcription, and cell differentiation (Planque N et al, J Biol Chem 2001 May 11; Kataoka K et al, J Biol Chem 2001, 276:819; Dutta S et al, Proc Natl Acad Sci USA. 2001, 98:1065). Using immunoprecipitation techniques as well as two yeast hybrid systems, one can identify proteins that associate with 98P7C3. Immunoprecipitates from cells expressing 98P7C3 and cells lacking 98P7C3 are compared for specific protein-protein associations. 98P7C3 may also associate with effector molecules, such as HLH domain-containing proteins. Studies comparing 98P7C3 positive and 98P7C3 negative cells as well as studies comparing unstimulated/resting cells and cells treated with epithelial cell activators, such as cytokines, growth factors, androgen and anti-integrin Ab reveal unique interactions. This and similar assays are used to identify and screen for molecules that interact with 98P7C3. When 98P7C3 associates with proteins or small molecules it is used as a target for diagnostic, prognostic, preventative and therapeutic purposes.

### Example 16

### 98P7C3 Monoclonal Antibody-mediated Inhibition of Prostate Tumors In Vivo

The significant expression of 98P7C3, in cancer tissues, together with its restrictive expression in normal tissues along with its expected cell surface expression makes 98P7C3 an excellent target for antibody therapy. Similarly, 98P7C3 is a target for T cell-based immunotherapy. Thus, the therapeutic efficacy of anti-98P7C3 mAbs in human prostate cancer xenograft mouse models is evaluated by using androgen-independent LAPC-4 and LAPC-9 xenografts (Craft, N., et al.,. Cancer Res, 1999. 59(19): p. 5030-6) and the androgen independent recombinant cell line PC3-98P7C3 (see, e.g., Kaighn, M. E., et al., Invest Urol, 1979. 17(1): p. 16-23).

Antibody efficacy on tumor growth and metastasis formation is studied, e.g., in a mouse orthotopic prostate cancer xenograft model. The antibodies can be unconjugated, as discussed in this Example, or can be conjugated to a therapeutic modality, as appreciated in the art. We demonstrate that anti-98P7C3 mAbs inhibit formation of both the androgen-dependent LAPC-9 and androgen-independent PC3-98P7C3 tumor xenografts. Anti-98P7C3 mAbs also retard the growth of established orthotopic tumors and prolonged survival of tumor-bearing mice. These results indicate the utility of anti-98P7C3 mAbs in the treatment of local and advanced stages of prostate cancer. (See, e.g., (Saffran, D., et al., PNAS 10:1073-1078 or www.pnas.org/cgi/doi/10.1073/pnas.051624698)

Administration of the anti-98P7C3 mAbs led to retardation of established orthotopic tumor growth and inhibition of metastasis to distant sites, resulting in a significant prolongation in the survival of tumor-bearing mice. These studies indicate that 98P7C3 as an attractive target for immunotherapy and demonstrate the therapeutic potential of anti-98P7C3 mAbs for the treatment of local and metastatic prostate cancer. This example demonstrates that unconjugated 98P7C3 monoclonal antibodies are effective to inhibit the growth of human prostate tumor xenografts grown in SCID mice; accordingly a combination of such efficacious monoclonal antibodies is also effective.

Tumor inhibition using multiple unconjugated 98P7C3 mAbs

Materials and Methods

98P7C3 Monoclonal Antibodies:

Monoclonal antibodies are raised against 98P7C3 as described in Example 6. The antibodies are characterized by ELISA, Western blot, FACS, and immunoprecipitation for their capacity to bind 98P7C3. Epitope mapping data for the anti-98P7C3 mAbs, as determined by ELISA and Western analysis, recognize epitopes on the 98P7C3 protein. Immunohistochemical analysis of prostate cancer tissues and cells with these antibodies is performed.

The monoclonal antibodies are purified from ascites or hybridoma tissue culture supernatants by Protein-G Sepharose chromatography, dialyzed against PBS, filter sterilized, and stored at −20° C. Protein determinations are performed by a Bradford assay (Bio-Rad, Hercules, Calif.). A therapeutic monoclonal antibody or a cocktail comprising a mixture of individual monoclonal antibodies is prepared and used for the treatment of mice receiving subcutaneous or orthotopic injections of LAPC-9 prostate tumor xenografts.

Prostate Cancer Xenografts and Cell Lines

The LAPC-9 xenograft, which expresses a wild-type androgen receptor and produces prostate-specific antigen (PSA), is passaged in 6- to 8-week-old male ICR-severe combined immunodeficient (SCID) mice (Taconic Farms) by s.c. trocar implant (Craft, N., et al., supra). Single-cell suspensions of LAPC-9 tumor cells are prepared as described in Craft, et al. The prostate carcinoma cell line PC3 (American Type Culture Collection) is maintained in DMEM supplemented with L-glutamine and 10% (vol/vol) FBS.

A PC3-98P7C3 cell population is generated by retroviral gene transfer as described in Hubert, R. S., et al, STEAP: a prostate-specific cell-surface antigen highly expressed in human prostate 20 tumors. Proc Natl Acad Sci USA, 1999. 96(25): p. 14523-8. Anti-98P7C3 staining is detected by using an FITC-conjugated goat anti-mouse antibody (Southern Biotechnology Associates) followed by analysis on a Coulter Epics-XL flow cytometer.

Xenograft Mouse Models

Subcutaneous (s.c.) tumors are generated by injection of 1×10⁶ LAPC-9, PC3, or PC3-98P7C3 cells mixed at a 1:1 dilution with Matrigel (Collaborative Research) in the right flank of male SCID mice. To test antibody efficacy on tumor formation, i.p. antibody injections are started on the same day as tumor-cell injections. As a control, mice are injected with either purified mouse IgG (ICN) or PBS; or a purified monoclonal antibody that recognizes an irrelevant antigen not expressed in human cells. In preliminary studies, no difference is found between mouse IgG or PBS on tumor growth. Tumor sizes are determined by vernier caliper measurements, and the tumor volume is calculated as length×width×height. Mice with s.c. tumors greater than 1.5 cm in diameter are sacrificed. PSA levels are determined by using a PSA ELISA kit (Anogen, Mississauga, Ontario). Circulating levels of anti-98P7C3 mAbs are determined by a capture ELISA kit (Bethyl Laboratories, Montgomery, Tex.). (See, e.g., (Saffran, D., et al., PNAS 10:1073-1078 or www.pnas.org/cgi/doi/10.1073/pnas.051624698)

Orthotopic injections are performed under anesthesia by using ketamine/xylazine. An incision is made through the abdominal muscles to expose the bladder and seminal vesicles, which then are delivered through the incision to expose the dorsal prostate. LAPC-9 cells (5×10⁵ ) mixed with Matrigel are injected into each dorsal lobe in a 10-μl volume. To monitor tumor growth, mice are bled on a weekly basis for determination of PSA levels. Based on the PSA levels, the mice are segregated into groups for the appropriate treatments. To test the effect of anti-98P7C3 mAbs on established orthotopic tumors, i.p. antibody injections are started when PSA levels reach 2-80 ng/ml.

Anti-98P7C3 mAbs Inhibit Growth of 98P7C3-Expressing Prostate-Cancer Tumors

We next test the effect of anti-98P7C3 mAbs on tumor formation by using the LAPC-9 orthotopic model. As compared with the s.c. tumor model, the orthotopic model, which requires injection of tumor cells directly in the mouse prostate, results in a local tumor growth, development of metastasis in distal sites, deterioration of mouse health, and subsequent death (Saffran, D., et al., PNAS supra; Fu, X., et al., Int J Cancer, 1992. 52(6): p. 987-90; Kubota, T., J Cell Biochem, 1994. 56(1): p. 4-8). The features make the orthotopic model more representative of human disease progression and allowed us to follow the therapeutic effect of InAbs on clinically relevant end points.

Accordingly, LAPC-9 tumor cells are injected into the mouse prostate, and 2 days later, the mice are segregated into two groups and treated with either up to 200 μg, usually 10-50 μg, of anti-98P7C3 Ab or PBS three times per week for two to five weeks. Mice are monitored weekly for circulating PSA levels as an indicator of tumor growth.

A major advantage of the orthotopic prostate-cancer model is the ability to study the development of metastases. Formation of metastasis in mice bearing established orthotopic tumors is studies by IHC analysis on lung sections using an antibody against a prostate-specific cell-surface protein STEAP expressed at high levels in LAPC-9 xenografts (Hubert, R. S., et al., Proc Natl Acad Sci USA, 1999. 96(25): p. 14523-8).

Mice bearing established orthotopic LAPC-9 tumors are administered 11 injections of either anti-98P7C3 mAb or PBS over a 4-week period. Mice in both groups are allowed to establish a high tumor burden (PSA levels greater than 300 ng/ml), to ensure a high frequency of metastasis formation in mouse lungs. Mice then are killed and their prostate and lungs are analyzed for the presence of LAPC-9 cells by anti-STEAP IHC analysis.

These studies demonstrate a broad anti-tumor efficacy of anti-98P7C3 antibodies on initiation and progression of prostate cancer in xenograft mouse models. Anti-98P7C3 antibodies inhibit tumor formation of both androgen-dependent and androgen-independent tumors as well as retarding the growth of already established tumors and prolong the survival of treated mice. Moreover, anti-98P7C3 mAbs demonstrate a dramatic inhibitory effect on the spread of local prostate tumor to distal sites, even in the presence of a large tumor burden. Thus, anti-98P7C3 mabs are efficacious on major clinically relevant end points/PSA levels (tumor growth), prolongation of survival, and health.

### Example 17

### Androven Regulation of 98P7C3

To determine if 98P7C3 is regulated by androgen, LAPC-4 AD and LAPC-9 AD cells are grown in charcoal-stripped medium and stimulated with the synthetic androgen mibolerone, for either 14 or 24 hours. Expression of 98P7C3 is studied before and after stimulation with mibolerone. The experimental samples are confirmed by testing for the expression of the androgen-regulated prostate cancer gene PSA. In another experiment, 98P7C3 expression is analyzed in LAPC-9 AD and LAPC-9 AI tumors grown in castrated mice. Only, androgen independent tumors will grow in castrated mice.

When 98P7C3 expression is regulated by androgen, 98P7C3 is a target for diagnostic, preventative and therapeutic purposes.

### Example 18

### Amino Acid Scale Profiles and Identification of Antigenic Regions of 98P7C3

FIGS. 15, 16, 17, and 18 depict graphically four amino acid profiles of the 98P7C3 amino acid sequence, each assessment available by accessing the ProtScale website (http://www.expasy.ch/cgi-bin/protscale.pl) on the ExPasy molecular biology server.

These profiles: FIG. 15, Hydropathicity (Kyte J., Doolittle R. F., 1982. J. Mol. Biol. 157:105-132); FIG. 16, Average Flexibility (Bhaskaran R., and Ponnuswamy P. K., 1988. Int. J. Pept. Protein Res. 32:242-255); FIG. 17, Beta-turn (Deleage, G., Roux B. 1987 Protein Engineering 1:289-294); and, FIG. 18, Percentage Accessible Residues (Janin J., 1979 Nature 277:491-492), and optionally others on the ProtScale website, were used to identify antigenic regions of the 98P7C3 protein. The above amino acid profiles of 98P7C3 are generated using the following ProtScale parameters for analysis: 1) a window size of 9; 2) 100% weight of the window edges compared to the window center; and, 3) amino acid profile values normalized to lie between 0 and 1.

Hydropathicity (FIG. 15) and Percentage Accessible Residues (FIG. 18) profiles are used to determine stretches of hydrophilic amino acids (i.e., values less than 0.5 on the Hydropathicity profile and values greater than 0.5 on the Percentage Accessible Residues profile); such regions are likely to be exposed to the aqueous environment and thus available for immune recognition.

Average Flexibility (FIG. 16) and Beta-turn (FIG. 17) profiles determine stretches of amino acids (i.e., values greater than 0.5 on the Beta-turn profile and Average Flexibility profile) that are not constrained in secondary structures such as beta sheets and alpha helices. Such regions are also more likely to be exposed on the protein and thus accessible to immune recognition.

Antigenic sequences of the 98P7C3 protein indicated, e.g., by the profiles set forth in FIG. 15, FIG. 16, FIG. 17, or FIG. 18 are used to design immunogens, peptides or nucleic acids that encode them, to generate therapeutic and diagnostic anti-98P7C3 antibodies.

### Example 19

### Tissue-targeted Therapeutic Strategies

Given the expression of 98P7C3 in select normal tissues and in various cancer tissues, strategies can be employed to direct therapeutic 98P7C3 molecules to specific target tissues, as appropriate to an individual therapeutic application. For example, therapeutic compositions containing 98P7C3-related molecules are used for intravesical administration to treat kidney or bladder cancer. To treat prostate cancer, in another example, a therapeutic composition is injected within the prostatic capsule or directly within the tumor. In yet another example, aerosol administration is used to target lung tissue. Therapeutic compositions containing 98P7C3-related molecules are used for intraluminal or intratumoral administration to treat colon cancer. Therapeutic compositions containing 98P7C3-related molecules are used for intratumoral administration to treat breast, bone, bladder or pancreatic cancer. Therapeutic compositions containing 98P7C3-related molecules are used for intraluminal or intratumoral administration to treat cervical, stomach or rectal cancer. Therapeutic compositions containing 98P7C3-related molecules are used for intramedullary administration to treat leukemia. Therapeutic compositions containing 98P7C3-related molecules are used for intratumoral administration or injection into the ovarian capsule to treat ovarian cancer. Optionally any of the above modalities comprise pharmaceutical excipients to minimize systemic adsorption. Other methods of targeting therapeutic compositions to specific tissues are known in the art.

### Example 20

### HLA Class I and Class II Binding Assays

HLA class I and class II binding assays using purified HLA molecules are performed in accordance with disclosed protocols (e.g., PCT publications WO 94/20127 and WO 94/03205; Sidney et al., Current Protocols in Immunology 18.3.1 (1998); Sidney, et al., J. Immunol. 154:247 (1995); Sette, et al., Mol. Immunol. 31:813 (1994)). Briefly, purified MHC molecules (5 to 500 nM) are incubated with various unlabeled peptide inhibitors and 1-10 nM ¹²⁵I-radiolabeled probe peptides as described. Following incubation, MHC-peptide complexes are separated from free peptide by gel filtration and the fraction of peptide bound is determined. Typically, in preliminary experiments, each MHC preparation is titered in the presence of fixed amounts of radiolabeled peptides to determine the concentration of HLA molecules necessary to bind 10-20% of the total radioactivity. All subsequent inhibition and direct binding assays are performed using these HLA concentrations.

Since under these conditions [label]<[HLA] and IC₅₀≧[HLA], the measured IC₅₀ values are reasonable approximations of the true K_{D} values. Peptide inhibitors are typically tested at concentrations ranging from 120 μg/ml to 1.2 ng/ml, and are tested in two to four completely independent experiments. To allow comparison of the data obtained in different experiments, a relative binding figure is calculated for each peptide by dividing the IC₅₀ of a positive control for inhibition by the IC₅₀ for each tested peptide (typically unlabeled versions of the radiolabeled probe peptide). For database purposes, and inter-experiment comparisons, relative binding values are compiled. These values can subsequently be converted back into IC₅₀ nM values by dividing the IC₅₀ nM of the positive controls for inhibition by the relative binding of the peptide of interest. This method of data compilations accurate and consistent for comparing peptides that have been tested on different days, or with different lots of purified MHC.

Binding assays as outlined above may be used to analyze HLA supermotif and/or HLA motif-bearing peptides.

### Example 21

### Identification of HLA Supermotif- and Motif-Bearing CTL Candidate Epitopes

HLA vaccine compositions of the invention can include multiple epitopes. The multiple epitopes can comprise multiple HLA supermotifs or motifs to achieve broad population coverage. This example illustrates the identification of supermotif- and motif-bearing epitopes for the inclusion in such a vaccine composition. Calculation of population coverage is performed using the strategy described below.

Computer searches and algorithms for identification of supermotif and/or motif-bearing epitopes

The searches performed to identify the motif-bearing peptide sequences disclosed herein employ the protein sequence data from the gene product of 98P7C3 set forth in FIGS. 2 and 3.

Computer searches for epitopes bearing HLA Class I or Class II supermotifs or motifs are performed as follows. All translated 98P7C3 protein sequences are analyzed using a text string search software program to identify potential peptide sequences containing appropriate HLA binding motifs; such programs are readily produced in accordance with information in the art in view of known motif/supermotif disclosures. Furthermore, such calculations can be made mentally.

Identified A2-, A3-, and DR-supermotif sequences are scored using polynomial algorithms to predict their capacity to bind to specific HLA-Class I or Class II molecules. These polynomial algorithms account for the impact of different amino acids at different positions, and are essentially based on the premise that the overall affinity (or ΔG) of peptide-HLA molecule interactions can be approximated as a linear polynomial function of the type:

“ΔG”=a₁ᵢ×a₂ᵢ×a₃ᵢ . . . ×aₙᵢ

where aⱼᵢ is a coefficient which represents the effect of the presence of a given amino acid (j) at a given position (i) along the sequence of a peptide of n amino acids. The crucial assumption of this method is that the effects at each position are essentially independent of each other (i.e., independent binding of individual side-chains). When residue j occurs at position i in the peptide, it is assumed to contribute a constant amount jᵢ to the free energy of binding of the peptide irrespective of the sequence of the rest of the peptide.

The method of derivation of specific algorithm coefficients has been described in Gulukota et al., J. Mol. Biol. 267:1258-126, 1997; (see also Sidney et al., Human Immunol. 45:79-93, 1996; and Southwood et al., J. Immunol. 160:3363-3373, 1998). Briefly, for all i positions, anchor and non-anchor alike, the geometric mean of the average relative binding (ARB) of all peptides carrying j is calculated relative to the remainder of the group, and used as the estimate of jᵢ. For Class II peptides, if multiple alignments are possible, only the highest scoring alignment is utilized, following an iterative procedure. To calculate an algorithm score of a given peptide in a test set, the ARB values corresponding to the sequence of the peptide are multiplied. If this product exceeds a chosen threshold, the peptide is predicted to bind. Appropriate thresholds are chosen as a function of the degree of stringency of prediction desired.

Selection of HLA-A2 suipertye cross-reactive peptides

Complete protein sequences from 98P7C3 are scanned utilizing motif identification software, to identify 8-, 9- 10- and 11-mer sequences containing the HLA-A2-supermotif main anchor specificity. Typically, these sequences are then scored using the protocol described above and the peptides corresponding to the positive-scoring sequences are synthesized and tested for their capacity to bind purified HLA-A*0201 molecules in vitro (HLA-A*0201 is considered a prototype A2 supertype molecule).

These peptides are then tested for the capacity to bind to additional A2-supertype molecules (A*0202, A*0203, A*0206, and A*6802). Peptides that bind to at least three of the five A2-supertype alleles tested are typically deemed A2-supertype cross-reactive binders. Preferred peptides bind at an affinity equal to or less than 500 nM to three or more HLA-A2 supertype molecules.

Selection of HLA-A3 supermotif-bearing epitopes

The 98P7C3 protein sequence scanned above is also examined for the presence of peptides with the HLA-A3-supermotif primary anchors. Peptides corresponding to the HLA A3 supermotif-bearing sequences are then synthesized and tested for binding to HLA-A*0301 and HLA-A*1101 molecules, the molecules encoded by the two most prevalent A3-supertype alleles. The peptides that bind at least one of the two alleles with binding affinities of ≦500 nM, often ≦200 nM, are then tested for binding cross-reactivity to the other common A3-supertype alleles (e.g., A*3101, A*3301, and A*6801) to identify those that can bind at least three of the five HLA-A3-supertype molecules tested.

Selection of HLA-B7 supermotif bearing epitopes

The 98P7C3 protein is also analyzed for the presence of 8-, 9- 10-, or 11-mer peptides with the HLA-B7-supermotif. Corresponding peptides are synthesized and tested for binding to HLA-B*0702, the molecule encoded by the most common B7-supertype allele (i.e., the prototype B7 supertype allele). Peptides binding B*0702 with IC₅₀ of ≦500 nM are identified using standard methods. These peptides are then tested for binding to other common B7-supertype molecules (e.g., B*3501, B*5101, B*5301, and B*5401). Peptides capable of binding to three or more of the five B7-supertype alleles tested are thereby identified.

Selection of A1 and A24 motif-bearing epitopes

To further increase population coverage, HLA-A1 and -A24 epitopes can also be incorporated into vaccine compositions. An analysis of the 98P7C3 protein can also be performed to identify HLA-A1- and A24-motif-containing sequences.

High affinity and/or cross-reactive binding epitopes that bear other motif and/or supermotifs are identified using analogous methodology.

### Example 22

### Confirmation of Immunogenicity

Cross-reactive candidate CTL A2-supermotif-bearing peptides that are identified as described herein are selected for in vitro immunogenicity testing. Testing is performed using the following methodology:

Target Cell Lines for Cellular Screening:

The .221A2.1 cell line, produced by transferring the HLA-A2.1 gene into the HLA-A, -B, -C null mutant human B-lymphoblastoid cell line 721.221, is used as the peptide-loaded target to measure activity of HLA-A2.1-restricted CTL. This cell line is grown in RPMI-1640 medium supplemented with antibiotics, sodium pyruvate, nonessential amino acids and 10% (v/v) heat inactivated FCS. Cells that express an antigen of interest, or transfectants comprising the gene encoding the antigen of interest, can be used as target cells to test the ability of peptide-specific CTLs to recognize endogenous antigen.

Primary CTL Induction Cultures:

Generation of Dendritic Cells (DC): PBMCs are thawed in RPMI with 30 μg/ml DNAse, washed twice and resuspended in complete medium (RPMI-1640 plus 5% AB human serum, non-essential amino acids, sodium pyruvate, L-glutamine and penicillin/streptomycin). The monocytes are purified by plating 10×10⁶ PBMC/well in a 6-well plate. After 2 hours at 37° C., the non-adherent cells are removed by gently shaking the plates and aspirating the supernatants. The wells are washed a total of three times with 3 ml RPMI to remove most of the non-adherent and loosely adherent cells. Three ml of complete medium containing 50 ng/ml of GM-CSF and 1,000 U/ml of IL-4 are then added to each well. THFα is added to the DCs on day 6 at 75 ng/ml and the cells are used for CTL induction cultures on day 7.

Induction of CTL with DC and Peptide: CD8+ T-cells are isolated by positive selection with Dynal immunomagnetic beads (Dynabeads® M-450) and the detacha-bead® reagent. Typically about 200-250×10⁶ PBMC are processed to obtain 24×10⁶ CD8⁺ T-cells (enough for a 48-well plate culture). Briefly, the PBMCs are thawed in RPMI with 30 μg/ml DNAse, washed once with PBS containing 1% human AB serum and resuspended in PBS/1% AB serum at a concentration of 20×10⁶ cells/ml. The magnetic beads are washed 3 times with PBS/AB serum, added to the cells (140 μl beads/20×10⁶ cells) and incubated for 1 hour at 4° C. with continuous mixing. The beads and cells are washed 4× with PBS/AB serum to remove the nonadherent cells and resuspended at 100×10⁶ cells/ml (based on the original cell number) in PBS/AB serum containing 100μl/ml detacha-bead® reagent and 30μg/ml DNAse. The mixture is incubated for 1 hour at room temperature with continuous mixing. The beads are washed again with PBS/AB/DNAse to collect the CD8+ T-cells. The DC are collected and centrifuged at 1300 rpm for 5-7 minutes, washed once with PBS with 1% BSA, counted and pulsed with 40 μg/m, of peptide at a cell concentration of 1-2×10⁶/ml in the presence of 3 μg/ml β₂-microglobulin for 4 hours at 20° C. The DC are then irradiated (4,200 rads), washed 1 time with medium and counted again.

Setting up induction cultures: 0.25 ml cytokine-generated DC (@1×10⁵ cells/ml) are co-cultured with 0.25 ml of CD8+ T-cells (@2×10⁶ cell/ml) in each well of a 48-well plate in the presence of 10 ng/ml of IL-7. Recombinant human IL-10 is added the next day at a final concentration of 10 ng/ml and rhuman IL-2 is added 48 hours later at 10 IU/ml.

Restimulation of the induction cultures with peptide-pulsed adherent cells: Seven and fourteen days after the primary induction, the cells are restimulated with peptide-pulsed adherent cells. The PBMCs are thawed and washed twice with RPMI and DNAse. The cells are resuspended at 5×10⁶ cells/ml and irradiated at ˜4200 rads. The PBMCs are plated at 2×10⁶ in 0.5 ml complete medium per well and incubated for 2 hours at 37° C. The plates are washed twice with RPMI by tapping the plate gently to remove the nonadherent cells and the adherent cells pulsed with 10 μg/ml of peptide in the presence of 3 μg/ml β₂ microglobulin in 0.25 ml RPMP/5% AB per well for 2 hours at 37° C. Peptide solution from each well is aspirated and the wells are washed once with RPMI. Most of the media is aspirated from the induction cultures (CD8+ cells) and brought to 0.5 ml with fresh media. The cells are then transferred to the wells containing the peptide-pulsed adherent cells. Twenty four hours later recombinant human IL-10 is added at a final concentration of 10 ng/mil and recombinant human IL2 is added the next day and again 2-3 days later at 50 IU/ml (Tsai et al., Critical Reviews in Immunology 18(1-2):65-75, 1998). Seven days later, the cultures are assayed for CTL activity in a ⁵¹Cr release assay. In some experiments the cultures are assayed for peptide-specific recognition in the in situ IFNγ ELISA at the time of the second restimulation followed by assay of endogenous recognition 7 days later. After expansion, activity is measured in both assays for a side-by-side comparison.

Measurement of CTL lytic activity by ⁵¹Cr release

Seven days after the second restimulation, cytotoxicity is determined in a standard (5 hr) ⁵¹Cr release assay by assaying individual wells at a single E:T. Peptide-pulsed targets are prepared by incubating the cells with 10 μg/ml peptide overnight at 37° C.

Adherent target cells are removed from culture flasks with trypsin-EDTA. Target cells are labeled with 200 μCi of ⁵¹Cr sodium chromate (Dupont, Wilmington, Del.) for 1 hour at 37° C. Labeled target cells are resuspended at 10⁶ per ml and diluted 1:10 with K562 cells at a concentration of 3.3×10⁶/ml (an NK-sensitive erythroblastoma cell line used to reduce non-specific lysis). Target cells (100 μl) and effectors (100 μl) are plated in 96 well round-bottom plates and incubated for 5 hours at 37° C. At that time, 100 μl of supernatant are collected from each well and percent lysis is determined according to the formula:

[(cpm of the test sample-cpm of the spontaneous ⁵¹Cr release sample)/(cpm of the maximal ⁵¹Cr release sample-cpm of the spontaneous ⁵¹Cr release sample)]×100.

Maximum and spontaneous release are determined by incubating the labeled targets with 1% Triton X-100 and media alone, respectively. A positive culture is defined as one in which the specific lysis (sample-background) is 10% or higher in the case of individual wells and is 15% or more at the two highest E:T ratios when expanded cultures are assayed.

In situ Measurement of Human IFNγ Production as an Indicator of Peptide-specific and Endogenous Recognition

Immulon 2 plates are coated with mouse anti-human IFNγ monoclonal antibody (4 μg/ml 0.1M NaHCO₃, pH8.2) overnight at 4° C. The plates are washed with Ca²⁺, Mg²⁺-free PBS/0.05% Tween 20 and blocked with PBS/10% FCS for two hours, after which the CTLs (100 μl/well) and targets (100 μl/well) are added to each well, leaving empty wells for the standards and blanks (which received media only). The target cells, either peptide-pulsed or endogenous targets, are used at a concentration of 1×10⁶ cells/ml. The plates are incubated for 48 hours at 37° C. with 5% CO₂.

Recombinant human IFNγ is added to the standard wells starting at 400 pg or 1200 pg/100 μl/well and the plate incubated for two hours at 37° C. The plates are washed and 100 μl of biotinylated mouse anti-human IFNγ monoclonal antibody (2 μg/ml in PBS/3%FCS/0.05% Tween 20) are added and incubated for 2 hours at room temperature. After washing again, 100 μl HRP-streptavidin (1:4000) are added and the plates incubated for one hour at room temperature. The plates are then washed 6× with wash buffer, 100 μl/well developing solution (TMB 1:1) are added, and the plates allowed to develop for 5-15 minutes. The reaction is stopped with 50 μl/well 1M H₃PO₄ and read at OD450. A culture is considered positive if it measured at least 50 pg of IFNγ/well above background and is twice the background level of expression.

CTL Expansion

Those cultures that demonstrate specific lytic activity against peptide-pulsed targets and/or tumor targets are expanded over a two week period with anti-CD3. Briefly, 5×10⁴ CD8+ cells are added to a T25 flask containing the following: 1×10⁶ irradiated (4,200 rad) PBMC (autologous or allogeneic) per ml, 2×10⁵ irradiated (8,000 rad) EBV-transformed cells per ml, and OKT3 (anti-CD3) at 30 ng per ml in RPMI-1640 containing 10% (v/v) human AB serum, non-essential amino acids, sodium pyruvate, 25 μM 2-mercaptoethanol, L-glutamine and penicillin/streptomycin. Recombinant human IL2 is added 24 hours later at a final concentration of 200 IU/ml and every three days thereafter with fresh media at 50 IU/ml. The cells are split if the cell concentration exceeds 1×10⁶/ml and the cultures are assayed between days 13 and 15 at E:T ratios of 30, 10, 3 and 1:1 in the ⁵¹Cr release assay or at 1×10⁶/ml in the in situ IFNγ assay using the same targets as before the expansion.

Cultures are expanded in the absence of anti-CD3⁺ as follows. Those cultures that demonstrate specific lytic activity against peptide and endogenous targets are selected and 5×10⁴ CD8⁺ cells are added to a T25 flask containing the following: 1×10⁶ autologous PBMC per ml which have been peptide-pulsed with 10 μg/ml peptide for two hours at 37° C. and irradiated (4,200 rad); 2×10⁵ irradiated (8,000 rad) EBV-transformed cells per ml RPMI-1640 containing 10%(v/v) human AB serum, non-essential AA, sodium pyruvate, 25 mM 2-ME, L-glutamine and gentamicin.

Immunogenicity of A2 supermotif-bearing peptides

A2-supermotif cross-reactive binding peptides are tested in the cellular assay for the ability to induce peptide-specific CTL in normal individuals. In this analysis, a peptide is typically considered to be an epitope if it induces peptide-specific CTLs in at least individuals, and preferably, also recognizes the endogenously expressed peptide.

Immunogenicity can also be confirmed using PBMCs isolated from patients bearing a tumor that expresses 98P7C3. Briefly, PBMCs are isolated from patients, re-stimulated with peptide-pulsed monocytes and assayed for the ability to recognize peptide-pulsed target cells as well as transfected cells endogenously expressing the antigen.

Evaluation of A*03/A11 immunogenicity

HLA-A3 supermotif-bearing cross-reactive binding peptides are also evaluated for immunogenicity using methodology analogous for that used to evaluate the immunogenicity of the HLA-A2 supermotif peptides.

Evaluation of B7 immunogenicity

Immunogenicity screening of the B7-supertype cross-reactive binding peptides identified as set forth herein are evaluated in a manner analogous to the evaluation of A2-and A3-supermotif-bearing peptides.

Peptides bearing other supermotifs/motifs, e.g., HLA-A1, HLA-A24 etc. are also evaluated using similar methodology

### Example 23

### Implementation of the Extended Supermotif to Improve the Binding Capacity of Native Epitopes by Creating Analogs

HLA motifs and supermotifs (comprising primary and/or secondary residues) are useful in the identification and preparation of highly cross-reactive native peptides, as demonstrated herein. Moreover, the definition of HLA motifs and supermotifs also allows one to engineer highly cross-reactive epitopes by identifying residues within a native peptide sequence which can be analoged to confer upon the peptide certain characteristics, e.g. greater cross-reactivity within the group of HLA molecules that comprise a supertype, and/or greater binding affinity for some or all of those HLA molecules. Examples of analoging peptides to exhibit modulated binding affinity are set forth in this example.

Analoging at Primary Anchor Residues

Peptide engineering strategies are implemented to further increase the cross-reactivity of the epitopes. For example, the main anchors of A2-supermotif-bearing peptides are altered, for example, to introduce a preferred L, I, V, or M at position 2, and I or V at the C-terminus.

To analyze the cross-reactivity of the analog peptides, each engineered analog is initially tested for binding to the prototype A2 supertype allele A*0201, then, if A*0201 binding capacity is maintained, for A2-supertype cross-reactivity.

Alternatively, a peptide is tested for binding to one or all supertype members and then analogued to modulate binding affinity to any one (or more) of the supertype members to add population coverage.

The selection of analogs for immunogenicity in a cellular screening analysis is typically further restricted by the capacity of the parent wild type (WT) peptide to bind at least weakly, i.e., bind at an IC50 of 500 nM or less, to three of more A2 supertype alleles. The rationale for this requirement is that the WT peptides must be present endogenously in sufficient quantity to be biologically relevant.

Analoged peptides have been shown to have increased immunogenicity and cross-reactivity by T cells specific for the parent epitope (see, e.g., Parkhurst et al., J. Immunol. 157:2539, 1996; and Pogue et al., Proc. Natl. Acad. Sci. USA 92:8166, 1995).

In the cellular screening of these peptide analogs, it is important to demonstrate that analog-specific CTLs are also able to recognize the wild-type peptide and, when possible, target cells that endogenously express the epitope.

Analoging of HLA-A3 and B7-supermotif-bearing peptides

Analogs of HLA-A3 supermotif-bearing epitopes are generated using strategies similar to those employed in analoging HLA-A2 supermotif-bearing peptides. For example, peptides binding to 3/5 of the A3-supertype molecules are engineered at primary anchor residues to possess a preferred residue (V, S, M, or A) at position 2.

The analog peptides are then tested for the ability to bind A*03 and A*11 (prototype A3 supertype alleles). Those peptides that demonstrate <500 nM binding capacity are then tested for A3-supertype cross-reactivity.

Similarly to the A2- and A3- motif bearing peptides, peptides binding 3 or more B7-supertype alleles can be improved, where possible, to achieve increased cross-reactive binding or greater binding affinity or binding half life. B7 supermotif-bearing peptides are, for example, engineered to possess a preferred residue (V, I, L, or F) at the C-terminal primary anchor position, as demonstrated by Sidney et al. (J. Immunol. 157:3480-3490, 1996).

Analoging at primary anchor residues of other motif and/or supermotif-bearing epitopes is performed in a like manner.

The analog peptides are then be tested for immunogenicity, typically in a cellular screening assay. Again, it is generally important to demonstrate that analog-specific CTLs are also able to recognize the wild-type peptide and, when possible, targets that endogenously express the epitope.

Analoging at Secondary Anchor Residues

Moreover, HLA supermotifs are of value in engineering highly cross-reactive peptides and/or peptides that bind HLA molecules with increased affinity by identifying particular residues at secondary anchor positions that are associated with such properties. For example, the binding capacity of a B7 supermotif-bearing peptide with an F residue at position 1 is analyzed. The peptide is then analoged to, for example, substitute L for F at position 1. The analoged peptide is evaluated for increased binding affinity, binding half life and/or increased cross-reactivity. Such a procedure identifies analoged peptides with enhanced properties.

Engineered analogs with sufficiently improved binding capacity or cross-reactivity can also be tested for immunogenicity in HLA-B7-transgenic mice, following for example, IFA immunization or lipopeptide immunization. Analogued peptides are additionally tested for the ability to stimulate a recall response using PBMC from patients with 98P7C3-expressing tumors.

Other analoguing strategies

Another form of peptide analoguing, unrelated to anchor positions, involves the substitution of a cysteine with α-amino butyric acid. Due to its chemical nature, cysteine has the propensity to form disulfide bridges and sufficiently alter the peptide structurally so as to reduce binding capacity. Substitution of α-amino butyric acid for cysteine not only alleviates this problem, but has been shown to improve binding and crossbinding capabilities in some instances (see, e.g., the review by Sette et al., In: Persistent Viral Infections, Eds. R. Ahmed and I. Chen, John Wiley & Sons, England, 1999).

Thus, by the use of single amino acid substitutions, the binding properties and/or cross-reactivity of peptide ligands for HLA supertype molecules can be modulated.

### Example 24

### Identification of 98P7C3-derived Sequences with HLA-DR Binding Motifs

Peptide epitopes bearing an HLA class II supermotif or motif are identified as outlined below using methodology similar to that described for HLA Class I peptides.

Selection of HLA-DR-supermotif-bearing epitopes

To identify 98P7C3-derived, HLA class II HTL epitopes, the 98P7C3 antigen is analyzed for the presence of sequences bearing an HLA-DR-motif or supermotif. Specifically, 15-mer sequences are selected comprising a DR-supermotif, comprising a 9-mer core, and three-residue N- and C-terminal flanking regions (15 amino acids total).

Protocols for predicting peptide binding to DR molecules have been developed (Southwood et al., J. Immunol. 160:3363-3373, 1998). These protocols, specific for individual DR molecules, allow the scoring, and ranking, of 9-mer core regions. Each protocol not only scores peptide sequences for the presence of DR-supermotif primary anchors (i.e., at position 1 and position 6) within a 9-mer core, but additionally evaluates sequences for the presence of secondary anchors. Using allele-specific selection tables (see, e.g., Southwood et al., ibid.), it has been found that these protocols efficiently select peptide sequences with a high probability of binding a particular DR molecule. Additionally, it has been found that performing these protocols in tandem, specifically those for DR1, DR4w4, and DR7, can efficiently select DR cross-reactive peptides.

The 98P7C3 -derived peptides identified above are tested for their binding capacity for various common HLA-DR molecules. All peptides are initially tested for binding to the DR molecules in the primary panel: DR1, DR4w4, and DR7. Peptides binding at least two of these three DR molecules are then tested for binding to DR2w2 β1, DR2w2β2, DR6w19, and DR9 molecules in secondary assays. Finally, peptides binding at least two of the four secondary panel DR molecules, and thus cumulatively at least four of seven different DR molecules, are screened for binding to DR4w15, DR5w11, and DR8w2 molecules in tertiary assays. Peptides binding at least seven of the ten DR molecules comprising the primary, secondary, and tertiary screening assays are considered cross-reactive DR binders. 98P7C3-derived peptides found to bind common HLA-DR alleles are of particular interest.

Selection of DR3 motif peptides

Because HLA-DR3 is an allele that is prevalent in Caucasian, Black, and Hispanic populations, DR3 binding capacity is a relevant criterion in the selection of HTL epitopes. Thus, peptides shown to be candidates may also be assayed for their DR3 binding capacity. However, in view of the binding specificity of the DR3 motif, peptides binding only to DR3 can also be considered as candidates for inclusion in a vaccine formulation.

To efficiently identify peptides that bind DR3, target 98P7C3 antigens are analyzed for sequences carrying one of the two DR3-specific binding motifs reported by Geluk et al. (J. Immunol. 152:5742-5748, 1994). The corresponding peptides are then synthesized and tested for the ability to bind DR3 with an affinity of 1 μm or better, i.e., less than 1 μM. Peptides are found that meet this binding criterion and qualify as HLA class II high affinity binders.

DR3 binding epitopes identified in this manner are included in vaccine compositions with DR supermotif-bearing peptide epitopes.

Similarly to the case of HLA class I motif-bearing peptides, the class II motif-bearing peptides are analoged to improve affinity or cross-reactivity. For example, aspartic acid at position 4 of the 9-mer core sequence is an optimal residue for DR3 binding, and substitution for that residue often improves DR 3 binding.

### Example 25

### Immunogenicity of 98P7C3-derived HTL Epitopes

This example determines immunogenic DR supermotif- and DR3 motif-bearing epitopes among those identified using the methodology set forth herein.

Immunogenicity of HTL epitopes are evaluated in a manner analogous to the determination of immunogenicity of CTL epitopes, by assessing the ability to stimulate HTL responses and/or by using appropriate transgenic mouse models. Immunogenicity is determined by screening for: 1.) in vitro primary induction using normal PBMC or 2.) recall responses from patients who have 98P7C3-expressing tumors.

### Example 26

### Calculation of Phenotypic Frequencies of HLA-supertypes in Various Ethnic Backgrounds to Determine Breadth of Population Coverage

This example illustrates the assessment of the breadth of population coverage of a vaccine composition comprised of multiple epitopes comprising multiple supermotifs and/or motifs.

In order to analyze population coverage, gene frequencies of HLA alleles are determined. Gene frequencies for each HLA allele are calculated from antigen or allele frequencies utilizing the binomial distribution formulae gf=1-(SQRT(1-af)) (see, e.g., Sidney et al., Human Immunol. 45:79-93, 1996). To obtain overall phenotypic frequencies, cumulative gene frequencies are calculated, and the cumulative antigen frequencies derived by the use of the inverse formula [af=1-(1-Cgf)2].

Where frequency data is not available at the level of DNA typing, correspondence to the serologically defined antigen frequencies is assumed. To obtain total potential supertype population coverage no linkage disequilibrium is assumed, and only alleles confirmed to belong to each of the supertypes are included (minimal estimates). Estimates of total potential coverage achieved by inter-loci combinations are made by adding to the A coverage the proportion of the non-A covered population that could be expected to be covered by the B alleles considered (e.g., total=A+B*(1-A)). Confirmed members of the A3-like supertype are A3, A11, A31, A*3301, and A*6801. Although the A3-like supertype may also include A34, A66, and A*7401, these alleles were not included in overall frequency calculations. Likewise, confirmed members of the A2-like supertype family are A*0201, A*0202, A*0203, A*0204, A*0205, A*0206, A*0207, A*6802, and A*6901. Finally, the B7-like supertype-confirmed alleles are: B7, B*3501-03, B51, B*5301, B*5401, B*5501-2, B*5601, B*6701, and B*7801 (potentially also B*1401, B*3504-06, B*4201, and B*5602).

Population coverage achieved by combining the A2-, A3- and B7-supertypes is approximately 86% in five major ethnic groups. Coverage may be extended by including peptides bearing the A1 and A24 motifs. On average, A1 is present in 12% and A24 in 29% of the population across five different major ethnic groups (Caucasian, North American Black, Chinese, Japanese, and Hispanic). Together, these alleles are represented with an average frequency of 39% in these same ethnic populations. The total coverage across the major ethnicities when A1 and A24 are combined with the coverage of the A2-, A3- and B7-supertype alleles is >95%. An analogous approach can be used to estimate population coverage achieved with combinations of class II motif-bearing epitopes.

Immunogenicity studies in humans (e.g., Bertoni et al., J. Clin. Invest. 100:503, 1997; Doolan et al., Immunity 7:97, 1997; and Threlkeld et al., J. Immunol. 159:1648, 1997) have shown that highly cross-reactive binding peptides are almost always recognized as epitopes. The use of highly cross-reactive binding peptides is an important selection criterion in identifying candidate epitopes for inclusion in a vaccine that is immunogenic in a diverse population.

With a sufficient number of epitopes (as disclosed herein and from the art), an average population coverage is predicted to be greater than 95% in each of five major ethnic populations. The game theory Monte Carlo simulation analysis, which is known in the art (see e.g., Osborne, M. J. and Rubinstein, A. “A course in game theory” MIT Press, 1994), can be used to estimate what percentage of the individuals in a population comprised of the Caucasian, North American Black, Japanese, Chinese, and Hispanic ethnic groups would recognize the vaccine epitopes described herein. A preferred percentage is 90%. A more preferred percentage is 95%.

### Example 27

### CTL Recognition of Endogenously Processed Antigens After Priming

This example determines that CTL induced by native or analoged peptide epitopes identified and selected as described herein recognize endogenously synthesized, i.e., native antigens.

Effector cells isolated from transgenic mice that are immunized with peptide epitopes, for example HLA-A2 supermotif-bearing epitopes, are re-stimulated in vitro using peptide-coated stimulator cells. Six days later, effector cells are assayed for cytotoxicity and the cell lines that contain peptide-specific cytotoxic activity are further re-stimulated. An additional six days later, these cell lines are tested for cytotoxic activity on 51Cr labeled Jurkat-A2.1/Kb target cells in the absence or presence of peptide, and also tested on 51Cr labeled target cells bearing the endogenously synthesized antigen, i.e. cells that are stably transfected with 98P7C3 expression vectors.

The results demonstrate that CTL lines obtained from animals primed with peptide epitope recognize endogenously synthesized 98P7C3 antigen. The choice of transgenic mouse model to be used for such an analysis depends upon the epitope(s) that are being evaluated. In addition to HLA-A*0201/Kb transgenic mice, several other transgenic mouse models including mice with human A11, which may also be used to evaluate A3 epitopes, and B7 alleles have been characterized and others (e.g., transgenic mice for HLA-A1 and A24) are being developed. HLA-DR1 and HLA-DR3 mouse models have also been developed, which may be used to evaluate HTL epitopes.

### Example 28

### Activity of CTL-HTL Conjugated Epitopes In Transgenic Mice

This example illustrates the induction of CTLs and HTLs in transgenic mice, by use of a 98P7C3-derived CTL and HTL peptide vaccine compositions. The vaccine composition used herein comprise peptides to be administered to a patient with a 98P7C3-expressing tumor. The peptide composition can comprise multiple CTL and/or HTL epitopes. The epitopes are identified using methodology as described herein. This example also illustrates that enhanced immunogenicity can be achieved by inclusion of one or more HTL epitopes in a CTL vaccine composition; such a peptide composition can comprise an HTL epitope conjugated to a CTL epitope. The CTL epitope can be one that binds to multiple HLA family members at an affinity of 500 nM or less, or analogs of that epitope. The peptides may be lipidated, if desired.

Immunization procedures: Immunization of transgenic mice is performed as described (Alexander et al., J. Immunol. 159:4753-4761, 1997). For example, A2/Kb mice, which are transgenic for the human HLA A2.1 allele and are used to assess the immunogenicity of HLA-A*0201 motif- or HLA-A2 supermotif-bearing epitopes, and are primed subcutaneously (base of the tail) with a 0.1 ml of peptide in Incomplete Freund's Adjuvant, or if the peptide composition is a lipidated CTL/HTL conjugate, in DMSO/saline, or if the peptide composition is a polypeptide, in PBS or Incomplete Freund's Adjuvant. Seven days after priming, splenocytes obtained from these animals are restimulated with syngeneic irradiated LPS-activated lymphoblasts coated with peptide.

Cell lines: Target cells for peptide-specific cytotoxicity assays are Jurkat cells transfected with the HLA-A2.1/K^{b} chimeric gene (e.g., Vitiello et al., J. Exp. Med. 173:1007, 1991)

In vitro CTL activation: One week after priming, spleen cells (30×10⁶ cells/flask) are co-cultured at 37° C. with syngeneic, irradiated (3000 rads), peptide coated lymphoblasts (10×10⁶ cells/flask) in 10 ml of culture medium/T25 flask. After six days, effector cells are harvested and assayed for cytotoxic activity.

Assay for cytotoxic activity: Target cells (1.0 to 1.5×10⁶) are incubated at 37° C. in the presence of 200 μl of 51Cr. After 60 minutes, cells are washed three times and resuspended in R10 medium. Peptide is added where required at a concentration of 1 μg/ml. For the assay, 104 51Cr-labeled target cells are added to different concentrations of effector cells (final volume of 200 μl) in U-bottom 96-well plates. After a six hour incubation period at 37° C., a 0.1 ml aliquot of supernatant is removed from each well and radioactivity is determined in a Micromedic automatic gamma counter. The percent specific lysis is determined by the formula: percent specific release=100×(experimental release−spontaneous release)/(maximum release−spontaneous release). To facilitate comparison between separate CTL assays run under the same conditions, % 51 Cr release data is expressed as lytic units/10⁶ cells. One lytic unit is arbitrarily defined as the number of effector cells required to achieve 30% lysis of 10,000 target cells in a six hour ⁵¹Cr release assay. To obtain specific lytic units/10⁶, the lytic units/10⁶ obtained in the absence of peptide is subtracted from the lytic units/10⁶ obtained in the presence of peptide. For example, if 30% ⁵¹Cr release is obtained at the effector (E): target (T) ratio of 50:1 (i.e., 5×10⁵ effector cells for 10,000 targets) in the absence of peptide and 5:1 (i.e., 5×10⁴ effector cells for 10,000 targets) in the presence of peptide, the specific lytic units would be: [(1/50,000)−(1/500,000)]×10⁶=18 LU.

The results are analyzed to assess the magnitude of the CTL responses of animals injected with the immunogenic CTL/HTL conjugate vaccine preparation and are compared to the magnitude of the CTL response achieved using, for example, CTL epitopes as outlined above in the Example entitled “Confirmation of Immunogenicity”. Analyses similar to this may be performed to evaluate the immunogenicity of peptide conjugates containing multiple CTL epitopes and/or multiple HTL epitopes. In accordance with these procedures, it is found that a CTL response is induced, and concomitantly that an HTL response is induced upon administration of such compositions.

### Example 29

### Selection of CTL and HTL Epitopes for Inclusion in a 98P7C3-specific Vaccine

This example illustrates a procedure for selecting peptide epitopes for vaccine compositions of the invention. The peptides in the composition can be in the form of a nucleic acid sequence, either single or one or more sequences (i.e., minigene) that encodes peptide(s), or can be single and/or polyepitopic peptides.

The following principles are utilized when selecting a plurality of epitopes for inclusion in a vaccine composition. Each of the following principles is balanced in order to make the selection.

Epitopes are selected which, upon administration, mimic immune responses that are correlated with 98P7C3 clearance. The number of epitopes used depends on observations of patients who spontaneously clear 98P7C3. For example, if it has been observed that patients who spontaneously clear 98P7C3 generate an immune response to at least three (3) from 98P7C3 antigen, then three or four (3-4) epitopes should be included for HLA class I. A similar rationale is used to determine HLA class II epitopes.

Epitopes are often selected that have a binding affinity of an IC50 of 500 mM or less for an HLA class I molecule, or for class II, an IC50 of 1000 nM or less; or HLA Class I peptides with high binding scores form the BIMAS web site, http://bimas.dcrt.nih.gov/.

In order to achieve broad coverage of the vaccine through out a diverse population, sufficient supermotif bearing peptides, or a sufficient array of allele-specific motif bearing peptides, are selected to give broad population coverage. In one embodiment, epitopes are selected to provide at least 80% population coverage. A Monte Carlo analysis, a statistical evaluation known in the art, can be employed to assess breadth, or redundancy, of population coverage.

When creating polyepitopic compositions, or a minigene that encodes same, it is typically desirable to generate the smallest peptide possible that encompasses the epitopes of interest. The principles employed are similar, if not the same, as those employed when selecting a peptide comprising nested epitopes. For example, a protein sequence for the vaccine composition is selected because it has maximal number of epitopes contained within the sequence, i.e., it has a high concentration of epitopes. Epitopes may be nested or overlapping (i.e., frame shifted relative to one another). For example, with overlapping epitopes, two 9-mer epitopes and one 10-mer epitope can be present in a 10 amino acid peptide. Each epitope can be exposed and bound by an HLA molecule upon administration of such a peptide. A multi-epitopic, peptide can be generated synthetically, recombinantly, or via cleavage from the native source. Alternatively, an analog can be made of this native sequence, whereby one or more of the epitopes comprise substitutions that alter the cross-reactivity and/or binding affinity properties of the polyepitopic peptide. Such a vaccine composition is administered for therapeutic or prophylactic purposes. This embodiment provides for the possibility that an as yet undiscovered aspect of immune system processing will apply to the native nested sequence and thereby facilitate the production of therapeutic or prophylactic immune response-inducing vaccine compositions. Additionally such an embodiment provides for the possibility of motif-bearing epitopes for an HLA makeup that is presently unknown. Furthermore, this embodiment (absent the creating of any analogs) directs the immune response to multiple peptide sequences that are actually present in 98P7C3, thus avoiding the need to evaluate any junctional epitopes. Lastly, the embodiment provides an economy of scale when producing nucleic acid vaccine compositions. Related to this embodiment, computer programs can be derived in accordance with principles in the art, which identify in a target sequence, the greatest number of epitopes per sequence length.

A vaccine composition comprised of selected peptides, when administered, is safe, efficacious, and elicits an immune response similar in magnitude to an immune response that controls or clears cells that bear or overexpress 98P7C3.

### Example 30

### Construction of “Minigene” Multi-Epitope DNA Plasmids

This example discusses the construction of a minigene expression plasmid. Minigene plasmids may, of course, contain various configurations of B cell, CTL and/or HTL epitopes or epitope analogs as described herein.

A minigene expression plasmid typically includes multiple CTL and HTL peptide epitopes. In the present example, HLA-A2, -A3, -B7 supermotif-bearing peptide epitopes and HLA-A1 and -A24 motif-bearing peptide epitopes are used in conjunction with DR supermotif-bearing epitopes and/or DR3 epitopes. HLA class I supermotif or motif-bearing peptide epitopes derived 98P7C3, are selected such that multiple supermotifs/motifs are represented to ensure broad population coverage. Similarly, HLA class II epitopes are selected from 98P7C3 to provide broad population coverage, i.e. both HLA DR-1-4-7 supermotif-bearing epitopes and HLA DR-3 motif-bearing epitopes are selected for inclusion in the minigene construct. The selected CTL and HTL epitopes are then incorporated into a minigene for expression in an expression vector.

Such a construct may additionally include sequences that direct the HTL epitopes to the endoplasmic reticulum. For example, the Ii protein may be fused to one or more HTL epitopes as described in the art, wherein the CLIP sequence of the Ii protein is removed and replaced with an HLA class II epitope sequence so that HLA class II epitope is directed to the endoplasmic reticulum, where the epitope binds to an HLA class II molecules.

This example illustrates the methods to be used for construction of a minigene-bearing expression plasmid. Other expression vectors that may be used for minigene compositions are available and known to those of skill in the art.

The minigene DNA plasmid of this example contains a consensus Kozak sequence and a consensus murine kappa Ig-light chain signal sequence followed by CTL and/or HTL epitopes selected in accordance with principles disclosed herein. The sequence encodes an open reading frame fused to the Myc and His antibody epitope tag coded for by the pcDNA 3.1 Myc-His vector.

Overlapping oligonucleotides that can, for example, average about 70 nucleotides in length with 15 nucleotide overlaps, are synthesized and HPLC-purified. The oligonucleotides encode the selected peptide epitopes as well as appropriate linker nucleotides, Kozak sequence, and signal sequence. The final multiepitope minigene is assembled by extending the overlapping oligonucleotides in three sets of reactions using PCR. A Perkin/Elmer 9600 PCR machine is used and a total of 30 cycles are performed using the following conditions: 95° C. for 15 sec, annealing temperature (5° below the lowest calculated Tm of each primer pair) for 30 sec, and 72° C. for 1 min.

For example, a minigene is prepared as follows. For a first PCR reaction, 5 μg of each of two oligonucleotides are annealed and extended: In an example using eight oligonucleotides, i.e., four pairs of primers, oligonucleotides 1+2, 3+4, 5+6, and 7+8 are combined in 100 μl reactions containing Pfu polymerase buffer (1×=10 mM KCL, 10 mM (NH4)₂SO₄, 20 mM Tris-chloride, pH 8.75, 2 mM MgSO₄, 0.1% Triton X-100, 100 μg/ml BSA), 0.25 mM each dNTP, and 2.5 U of Pfu polymerase. The full-length dimer products are gel-purified, and two reactions containing the product of 1+2 and 3+4, and the product of 5+6 and 7+8 are mixed, annealed, and extended for 10 cycles. Half of the two reactions are then mixed, and 5 cycles of annealing and extension carried out before flanking primers are added to amplify the full length product. The full-length product is gel-purified and cloned into pCR-blunt (Invitrogen) and individual clones are screened by sequencing.

### Example 31

### The Plasmid Construct and the Degree to Which It Induces Immunogenicity

The degree to which a plasmid construct, for example a plasmid constructed in accordance with the previous Example, is able to induce immunogenicity is evaluated in vitro by testing for epitope presentation by APC following transduction or transfection of the APC with an epitope-expressing nucleic acid construct. Such a study determines “antigenicity” and allows the use of human APC. The assay determines the ability of the epitope to be presented by the APC in a context that is recognized by a T cell by quantifying the density of epitope-HLA class I complexes on the cell surface. Quantitation can be performed by directly measuring the amount of peptide eluted from the APC (see, e.g., Sijts et al., J. Immunol. 156:683-692, 1996; Demotz et al., Nature 342:682-684, 1989); or the number of peptide-HLA class I complexes can be estimated by measuring the amount of lysis or lymphokine release induced by diseased or transfected target cells, and then determining the concentration of peptide necessary to obtain equivalent levels of lysis or lymphokine release (see, e.g., Kageyama et al., J. Immunol. 154:567-576, 1995).

Alternatively, immunogenicity is evaluated through in vivo injections into mice and subsequent in vitro assessment of CTL and HTL activity, which are analyzed using cytotoxicity and proliferation assays, respectively, as detailed e.g., in Alexander et al., Immunity 1:751-761, 1994.

For example, to assess the capacity of a DNA minigene construct containing at least one HLA-A2 supermotif peptide to induce CTLs in vivo, HLA-A2.₁/K^{b} transgenic mice, for example, are immunized intramuscularly with 100 μg of naked cDNA. As a means of comparing the level of CTLs induced by cDNA immunization, a control group of animals is also immunized with an actual peptide composition that comprises multiple epitopes synthesized as a single polypeptide as they would be encoded by the minigene.

Splenocytes from immunized animals are stimulated twice with each of the respective compositions (peptide epitopes encoded in the minigene or the polyepitopic peptide), then assayed for peptide-specific cytotoxic activity in a ⁵¹Cr release assay. The results indicate the magnitude of the CTL response directed against the A2-restricted epitope, thus indicating the in vivo immunogenicity of the minigene vaccine and polyepitopic vaccine.

It is, therefore, found that the minigene elicits immune responses directed toward the HLA-A2 supermotif peptide epitopes as does the polyepitopic peptide vaccine. A similar analysis is also performed using other HLA-A3 and HLA-B7 transgenic mouse models to assess CTL induction by HLA-A3 and HLA-B7 motif or supermotif epitopes, whereby it is also found that the minigene elicits appropriate immune responses directed toward the provided epitopes.

To assess the capacity of a class II epitope-encoding minigene to induce HTLs in vivo, DR transgenic mice, or for those epitopes that cross react with the appropriate mouse MHC molecule, I-Ab-restricted mice, for example, are immunized intramuscularly with 100 μg of plasmid DNA. As a means of comparing the level of HTLs induced by DNA immunization, a group of control animals is also immunized with an actual peptide composition emulsified in complete Freund's adjuvant. CD4+ T cells, i.e. HTLs, are purified from splenocytes of immunized animals and stimulated with each of the respective compositions (peptides encoded in the minigene). The HTL response is measured using a ³H-thymidine incorporation proliferation assay, (see, e.g., Alexander et al. Immunity 1:751-761, 1994). The results indicate the magnitude of the HTL response, thus demonstrating the in vivo immunogenicity of the minigene.

DNA minigenes, constructed as described in the previous Example, can also be evaluated as a vaccine in combination with a boosting agent using a prime boost protocol. The boosting agent can consist of recombinant protein (e.g., Barnett et al., Aids Res. and Human Retroviruses 14, Supplement 3:S299-S309, 1998) or recombinant vaccinia, for example, expressing a minigene or DNA encoding the complete protein of interest (see, e.g., Hanke et al., Vaccine 16:439-445, 1998; Sedegah et al., Proc. Natl. Acad. Sci USA 95:7648-53, 1998; Hanke and McMichael, Immunol. Letters 66:177-181, 1999; and Robinson et al., Nature Med. 5:526-34, 1999).

For example, the efficacy of the DNA minigene used in a prime boost protocol is initially evaluated in transgenic mice. In this example, A2. 1/Kb transgenic mice are immunized IM with 100 μg of a DNA minigene encoding the immunogenic peptides including at least one HLA-A2 supermotif-bearing peptide. After an incubation period (ranging from 3-9 weeks), the mice are boosted IP with 107 pfu/mouse of a recombinant vaccinia virus expressing the same sequence encoded by the DNA minigene. Control mice are immunized with 100 μg of DNA or recombinant vaccinia without the minigene sequence, or with DNA encoding the minigene, but without the vaccinia boost. After an additional incubation period of two weeks, splenocytes from the mice are immediately assayed for peptide-specific activity in an ELISPOT assay. Additionally, splenocytes are stimulated in vitro with the A2-restricted peptide epitopes encoded in the minigene and recombinant vaccinia, then assayed for peptide-specific activity in an alpha, beta and/or gamma IFN ELISA.

It is found that the minigene utilized in a prime-boost protocol elicits greater immune responses toward the HLA-A2 supermotif peptides than with DNA alone. Such an analysis can also be performed using HLA-A11 or HLA-B7 transgenic mouse models to assess CTL induction by HLA-A3 or HLA-B7 motif or supermotif epitopes. The use of prime boost protocols in humans is described below in the Example entitled “Induction of CTL Responses Using a Prime Boost Protocol.”

### Example 32

### Peptide Composition for Prophylactic Uses

Vaccine compositions of the present invention can be used to prevent 98P7C3 expression in persons who are at risk for tumors that bear this antigen. For example, a polyepitopic peptide epitope composition (or a nucleic acid comprising the same) containing multiple CTL and HTL epitopes such as those selected in the above Examples, which are also selected to target greater than 80% of the population, is administered to individuals at risk for a 98P7C3-associated tumor.

For example, a peptide-based composition is provided as a single polypeptide that encompasses multiple epitopes. The vaccine is typically administered in a physiological solution that comprises an adjuvant, such as Incomplete Freunds Adjuvant. The dose of peptide for the initial immunization is from about 1 to about 50,000 μg, generally 100-5,000 μg, for a 70 kg patient. The initial administration of vaccine is followed by booster dosages at 4 weeks followed by evaluation of the magnitude of the immune response in the patient, by techniques that determine the presence of epitope-specific CTL populations in a PBMC sample. Additional booster doses are administered as required. The composition is found to be both safe and efficacious as a prophylaxis against 98P7C3-associated disease.

Alternatively, a composition typically comprising transfecting agents is used for the administration of a nucleic acid-based vaccine in accordance with methodologies known in the art and disclosed herein.

### Example 33

### Polyepitopic Vaccine Compositions Derived from Native 98P7C3 Sequences

A native 98P7C3 polyprotein sequence is screened, preferably using computer algorithms defined for each class I and/or class II supermotif or motif, to identify “relatively short” regions of the polyprotein that comprise multiple epitopes. The “relatively short” regions are preferably less in length than an entire native antigen. This relatively short sequence that contains multiple distinct or overlapping, “nested” epitopes is selected; it can be used to generate a minigene construct. The construct is engineered to express the peptide, which corresponds to the native protein sequence. The “relatively short” peptide is generally less than 250 amino acids in length, often less than 100 amino acids in length, preferably less than 75 amino acids in length, and more preferably less than 50 amino acids in length. The protein sequence of the vaccine composition is selected because it has maximal number of epitopes contained within the sequence, i.e., it has a high concentration of epitopes. As noted herein, epitope motifs may be nested or overlapping (i.e., frame shifted relative to one another). For example, with overlapping epitopes, two 9-mer epitopes and one 1 0-mer epitope can be present in a 10 amino acid peptide. Such a vaccine composition is administered for therapeutic or prophylactic purposes.

The vaccine composition will include, for example, multiple CTL epitopes from 98P7C3 antigen and at least one HTL epitope. This polyepitopic native sequence is administered either as a peptide or as a nucleic acid sequence which encodes the peptide. Alternatively, an analog can be made of this native sequence, whereby one or more of the epitopes comprise substitutions that alter the cross-reactivity and/or binding affinity properties of the polyepitopic peptide.

The embodiment of this example provides for the possibility that an as yet undiscovered aspect of immune system processing will apply to the native nested sequence and thereby facilitate the production of therapeutic or prophylactic immune response-inducing vaccine compositions. Additionally such an embodiment provides for the possibility of motif-bearing epitopes for an HLA makeup that is presently unknown. Furthermore, this embodiment (excluding an analoged embodiment) directs the immune response to multiple peptide sequences that are actually present in native 98P7C3, thus avoiding the need to evaluate any junctional epitopes. Lastly, the embodiment provides an economy of scale when producing peptide or nucleic acid vaccine compositions.

Related to this embodiment, computer programs are available in the art which can be used to identify in a target sequence, the greatest number of epitopes per sequence length.

### Example 34

### Polyepitopic Vaccine Compositions From Multiple Antigens

The 98P7C3 peptide epitopes of the present invention are used in conjunction with epitopes from other target tumor-associated antigens, to create a vaccine composition that is useful for the prevention or treatment of cancer that expresses 98P7C3 and such other antigens. For example, a vaccine composition can be provided as a single polypeptide that incorporates multiple epitopes from 98P7C3 as well as tumor-associated antigens that are often expressed with a target cancer associated with 98P7C3 expression, or can be administered as a composition comprising a cocktail of one or more discrete epitopes. Alternatively, the vaccine can be administered as a minigene construct or as dendritic cells which have been loaded with the peptide epitopes in vitro.

### Example 35

### Use of Peptides to Evaluate an Immune Response

Peptides of the invention may be used to analyze an immune response for the presence of specific antibodies, CTL or HTL directed to 98P7C3. Such an analysis can be performed in a manner described by Ogg et al., Science 279:2103-2106, 1998. In this Example, peptides in accordance with the invention are used as a reagent for diagnostic or prognostic purposes, not as an immunogen.

In this example highly sensitive human leukocyte antigen tetrameric complexes (“tetramers”) are used for a cross-sectional analysis of, for example, 98P7C3 HLA-A*0201-specific CTL frequencies from HLA A*0201-positive individuals at different stages of disease or following immunization comprising a 98P7C3 peptide containing an A*0201 motif. Tetrameric complexes are synthesized as described (Musey et al., N. Engl. J. Med. 337:1267, 1997). Briefly, purified HLA heavy chain (A*0201 in this example) and P2-microglobulin are synthesized by means of a prokaryotic expression system. The heavy chain is modified by deletion of the transmembrane-cytosolic tail and COOH-terminal addition of a sequence containing a BirA enzymatic biotinylation site. The heavy chain, β2-microglobulin, and peptide are refolded by dilution. The 45-kD refolded product is isolated by fast protein liquid chromatography and then biotinylated by BirA in the presence of biotin (Sigma, St. Louis, Mo.), adenosine 5′triphosphate and magnesium. Streptavidin-phycoerythrin conjugate is added in a 1:4 molar ratio, and the tetrameric product is concentrated to 1 mg/ml. The resulting product is referred to as tetramer-phycoerythrin.

For the analysis of patient blood samples, approximately one million PBMCs are centrifuged at 300 g for 5 minutes and resuspended in 50 μl of cold phosphate-buffered saline. Tri-color analysis is performed with the tetramer-phycoerythrin, along with anti-CD8-Tricolor, and anti-CD38. The PBMCs are incubated with tetramer and antibodies on ice for 30 to 60 min and then washed twice before formaldehyde fixation. Gates are applied to contain >99.98% of control samples. Controls for the tetramers include both A*0201-negative individuals and A*0201-positive non-diseased donors. The percentage of cells stained with the tetramer is then determined by flow cytometry. The results indicate the number of cells in the PBMC sample that contain epitope-restricted CTLs, thereby readily indicating the extent of immune response to the 98P7C3 epitope, and thus the status of exposure to 98P7C3, or exposure to a vaccine that elicits a protective or therapeutic response.

### Example 36

### Use of Peptide Epitopes to Evaluate Recall Responses

The peptide epitopes of the invention are used as reagents to evaluate T cell responses, such as acute or recall responses, in patients. Such an analysis may be performed on patients who have recovered from 98P7C3-associated disease or who have been vaccinated with a 98P7C3 vaccine.

For example, the class I restricted CTL response of persons who have been vaccinated may be analyzed. The vaccine may be any 98P7C3 vaccine. PBMC are collected from vaccinated individuals and HLA typed. Appropriate peptide epitopes of the invention that, optimally, bear supermotifs to provide cross-reactivity with multiple HLA supertype family members, are then used for analysis of samples derived from individuals who bear that HLA type.

PBMC from vaccinated individuals are separated on Ficoll-Histopaque density gradients (Sigma Chemical Co., St. Louis, Mo.), washed three times in HBSS (GIBCO Laboratories), resuspended in RPMI-1640 (GIBCO Laboratories) supplemented with L-glutamine (2 mM), penicillin (50 U/ml), streptomycin (50 μg/ml), and Hepes (10 mM) containing 10% heat-inactivated human AB serum (complete RPMI) and plated using microculture formats. A synthetic peptide comprising an epitope of the invention is added at 10 μg/ml to each well and HBV core 128-140 epitope is added at 1 μg/ml to each well as a source of T cell help during the first week of stimulation.

In the microculture format, 4×10⁵ PBMC are stimulated with peptide in 8 replicate cultures in 96-well round bottom plate in 100 μl/well of complete RPMI. On days 3 and 10, 100 μl of complete RPMI and 20 U/ml final concentration of rIL-2 are added to each well. On day 7 the cultures are transferred into a 96-well flat-bottom plate and restimulated with peptide, rIL-2 and 10⁵ irradiated (3,000 rad) autologous feeder cells. The cultures are tested for cytotoxic activity on day 14. A positive CTL response requires two or more of the eight replicate cultures to display greater than 10% specific ⁵¹Cr release, based on comparison with non-diseased control subjects as previously described (Rehermann, et al., Nature Med. 2:1104, 1108, 1996; Rehermann et al., J Clin. Invest. 97:1655-1665, 1996; and Rehermann et al. J. Clin. Invest. 98:1432-1440, 1996).

Target cell lines are autologous and allogeneic EBV-transformed B-LCL that are either purchased from the American Society for Histocompatibility and Immunogenetics (ASHI, Boston, Mass.) or established from the pool of patients as described (Guilhot, et al. J. Virol 66:2670-2678, 1992).

Cytotoxicity assays are performed in the following manner. Target cells consist of either allogeneic HLA-matched or autologous EBV-transformed B lymphoblastoid cell line that are incubated overnight with the synthetic peptide epitope of the invention at 10 μM, and labeled with 100 μCi of ⁵¹Cr (Amersham Corp., Arlington Heights, Ill.) for 1 hour after which they are washed four times with HBSS.

Cytolytic activity is determined in a standard 4-h, split well ⁵¹Cr release assay using U-bottomed 96 well plates containing 3,000 targets/well. Stimulated PBMC are tested at effector/target (E/T) ratios of 20-50:1 on day 14. Percent cytotoxicity is determined from the formula: 100×[(experimental release-spontaneous release)/maximum release-spontaneous release)]. Maximum release is determined by lysis of targets by detergent (2% Triton X-100; Sigma Chemical Co., St. Louis, Mo.). Spontaneous release is <25% of maximum release for all experiments.

The results of such an analysis indicate the extent to which HLA-restricted CTL populations have been stimulated by previous exposure to 98P7C3 or a 98P7C3 vaccine.

Similarly, Class II restricted HTL responses may also be analyzed. Purified PBMC are cultured in a 96-well flat bottom plate at a density of 1.5×10⁵ cells/well and are stimulated with 10 μg/ml synthetic peptide of the invention, whole 98P7C3 antigen, or PHA. Cells are routinely plated in replicates of 4-6 wells for each condition. After seven days of culture, the medium is removed and replaced with fresh medium containing 10 U/ml IL-2. Two days later, 1 μCi ³H-thymidine is added to each well and incubation is continued for an additional 18 hours. Cellular DNA is then harvested on glass fiber mats and analyzed for ³H-thymidine incorporation. Antigen-specific T cell proliferation is calculated as the ratio of ³H-thymidine incorporation in the presence of antigen divided by the ³H-thymidine incorporation in the absence of antigen.

### Example 37

### Induction of Specific CTL Response In Humans

A human clinical trial for an immunogenic composition comprising CTL and HTL epitopes of the invention is set up as an IND Phase I, dose escalation study and carried out as a randomized, double-blind, placebo-controlled trial. Such a trial is designed, for example, as follows:

A total of about 27 individuals are enrolled and divided into 3 groups:

Group I: 3 subjects are injected with placebo and 6 subjects are injected with 5 μg of peptide composition;

Group II: 3 subjects are injected with placebo and 6 subjects are injected with 50 μg peptide composition;

Group III: 3 subjects are injected with placebo and 6 subjects are injected with 500 μg of peptide composition.

After 4 weeks following the first injection, all subjects receive a booster inoculation at the same dosage.

The endpoints measured in this study relate to the safety and tolerability of the peptide composition as well as its immunogenicity. Cellular immune responses to the peptide composition are an index of the intrinsic activity of this the peptide composition, and can therefore be viewed as a measure of biological efficacy. The following summarize the clinical and laboratory data that relate to safety and efficacy endpoints.

Safety: The incidence of adverse events is monitored in the placebo and drug treatment group and assessed in terms of degree and reversibility.

Evaluation of Vaccine Efficacy: For evaluation of vaccine efficacy, subjects are bled before and after injection. Peripheral blood mononuclear cells are isolated from fresh heparinized blood by Ficoll-Hypaque density gradient centrifugation, aliquoted in freezing media and stored frozen. Samples are assayed for CTL and HTL activity.

The vaccine is found to be both safe and efficacious.

### Example 38

### Phase II Trials In Patients Expressing 98P7C3

Phase II trials are performed to study the effect of administering the CTL-HTL peptide compositions to patients having cancer that expresses 98P7C3. The main objectives of the trial are to determine an effective dose and regimen for inducing CTLs in cancer patients that express 98P7C3, to establish the safety of inducing a CTL and HTL response in these patients, and to see to what extent activation of CTLs improves the clinical picture of these patients, as manifested, e.g., by the reduction and/or shrinking of lesions. Such a study is designed, for example, as follows:

The studies are performed in multiple centers. The trial design is an open-label, uncontrolled, dose escalation protocol wherein the peptide composition is administered as a single dose followed six weeks later by a single booster shot of the same dose. The dosages are 50, 500 and 5,000 micrograms per injection. Drug-associated adverse effects (severity and reversibility) are recorded.

There are three patient groupings. The first group is injected with 50 micrograms of the peptide composition and the second and third groups with 500 and 5,000 micrograms of peptide composition, respectively. The patients within each group range in age from 21-65 and represent diverse ethnic backgrounds. All of them have a tumor that expresses 98P7C3.

Clinical manifestations or antigen-specific T-cell responses are monitored to assess the effects of administering the peptide compositions. The vaccine composition is found to be both safe and efficacious in the treatment of 98P7C3-associated disease.

### Example 39

### Induction of CTL Responses Using a Prime Boost Protocol

A prime boost protocol similar in its underlying principle to that used to evaluate the efficacy of a DNA vaccine in transgenic mice, such as described above in the Example entitled “The Plasmid Construct and the Degree to Which It Induces Immunogenicity,” can also be used for the administration of the vaccine to humans. Such a vaccine regimen can include an initial administration of, for example, naked DNA followed by a boost using recombinant virus encoding the vaccine, or recombinant protein/polypeptide or a peptide mixture administered in an adjuvant.

For example, the initial immunization may be performed using an expression vector, such as that constructed in the Example entitled “Construction of ‘Minigene’ Multi-Epitope DNA Plasmids” in the form of naked nucleic acid administered IM (or SC or ID) in the amounts of 0.5-5 mg at multiple sites. The nucleic acid (0.1 to 1000 μg) can also be administered using a gene gun. Following an incubation period of 3-4 weeks, a booster dose is then administered. The booster can be recombinant fowlpox virus administered at a dose of 5-10⁷ to 5×10⁹ pfu. An alternative recombinant virus, such as an MVA, canarypox, adenovirus, or adeno-associated virus, can also be used for the booster, or the polyepitopic protein or a mixture of the peptides can be administered. For evaluation of vaccine efficacy, patient blood samples are obtained before immunization as well as at intervals following administration of the initial vaccine and booster doses of the vaccine. Peripheral blood mononuclear cells are isolated from fresh heparinized blood by Ficoll-Hypaque density gradient centrifugation, aliquoted in freezing media and stored frozen. Samples are assayed for CTL and HTL activity.

Analysis of the results indicates that a magnitude of response sufficient to achieve a therapeutic or protective immunity against 98P7C3 is generated.

### Example 40

### Administration of Vaccine Compositions Using Dendritic Cells (DC)

Vaccines comprising peptide epitopes of the invention can be administered using APCs, or “professional” APCs such as DC. In this example, peptide-pulsed DC are administered to a patient to stimulate a CTL response in vivo. In this method, dendritic cells are isolated, expanded, and pulsed with a vaccine comprising peptide CTL and HTL epitopes of the invention. The dendritic cells are infused back into the patient to elicit CTL and HTL responses in vivo. The induced CTL and HTL then destroy or facilitate destruction, respectively, of the target cells that bear the 98P7C3 protein from which the epitopes in the vaccine are derived.

For example, a cocktail of epitope-comprising peptides is administered ex vivo to PBMC, or isolated DC therefrom. A pharmaceutical to facilitate harvesting of DC can be used, such as Progenipoietin™ (Monsanto, St. Louis, Mo.) or GM-CSF/IL-4. After pulsing the DC with peptides, and prior to reinfusion into patients, the DC are washed to remove unbound peptides.

As appreciated clinically, and readily determined by one of skill based on clinical outcomes, the number of DC reinfused into the patient can vary (see, e.g., Nature Med. 4:328, 1998; Nature Med. 2:52, 1996 and Prostate 32:272, 1997). Although 2-50×10⁶ DC per patient are typically administered, larger number of DC, such as 10⁷ or 10⁸ can also be provided. Such cell populations typically contain between 50-90% DC.

In some embodiments, peptide-loaded PBMC are injected into patients without purification of the DC. For example, PBMC generated after treatment with an agent such as Progenipoietin™ are injected into patients without purification of the DC. The total number of PBMC that are administered often ranges from 10⁸ to 10¹⁰. Generally, the cell doses injected into patients is based on the percentage of DC in the blood of each patient, as determined, for example, by immunofluorescence analysis with specific anti-DC antibodies. Thus, for example, if Progenipoietin™ mobilizes 2% DC in the peripheral blood of a given patient, and that patient is to receive 5×10⁶ DC, then the patient will be injected with a total of 2.5×10⁸ peptide-loaded PBMC. The percent DC mobilized by an agent such as Progenipoietin™ is typically estimated to be between 2-10%, but can vary as appreciated by one of skill in the art.

Ex vivo activation of CTL/HTL responses

Alternatively, ex vivo CTL or HTL responses to 98P7C3 antigens can be induced by incubating, in tissue culture, the patient's, or genetically compatible, CTL or HTL precursor cells together with a source of APC, such as DC, and immunogenic peptides. After an appropriate incubation time (typically about 7-28 days), in which the precursor cells are activated and expanded into effector cells, the cells are infused into the patient, where they will destroy (CTL) or facilitate destruction (HTL) of their specific target cells, i.e., tumor cells.

### Example 41

### Alternative Method of Identifying Motif-Bearing Peptides

Another method of identifying motif-bearing peptides is to elute them from cells bearing defined MHC molecules. For example, EBV transformed B cell lines used for tissue typing have been extensively characterized to determine which HLA molecules they express. In certain cases these cells express only a single type of HLA molecule. These cells can be transfected with nucleic acids that express the antigen of interest, e.g. 98P7C3. Peptides produced by endogenous antigen processing of peptides produced as a result of transfection will then bind to HLA molecules within the cell and be transported and displayed on the cell's surface. Peptides are then eluted from the HLA molecules by exposure to mild acid conditions and their amino acid sequence determined, e.g., by mass spectral analysis (e.g., Kubo et al., J. Immunol. 152:3913, 1994). Because the majority of peptides that bind a particular HLA molecule are motif-bearing, this is an alternative modality for obtaining the motif-bearing peptides correlated with the particular HLA molecule expressed on the cell.

Alternatively, cell lines that do not express endogenous HLA molecules can be transfected with an expression construct encoding a single HLA allele. These cells can then be used as described, i.e., they can then be transfected with nucleic acids that encode 98P7C3 to isolate peptides corresponding to 98P7C3 that have been presented on the cell surface. Peptides obtained from such an analysis will bear motif(s) that correspond to binding to the single HLA allele that is expressed in the cell.

As appreciated by one in the art, one can perform a similar analysis on a cell bearing more than one HLA allele and subsequently determine peptides specific for each HLA allele expressed. Moreover, one of skill would also recognize that means other than transfection, such as loading with a protein antigen, can be used to provide a source of antigen to the cell.

### Example 42

### Complementary Polynucleotides

Sequences complementary to the 98P7C3-encoding sequences, or any parts thereof, are used to detect, decrease, or inhibit expression of naturally occurring 98P7C3. Although use of oligonucleotides comprising from about 15 to 30 base pairs is described, essentially the same procedure is used with smaller or with larger sequence fragments. Appropriate oligonucleotides are designed using, e.g., OLIGO 4.06 software (National Biosciences) and the coding sequence of 98P7C3. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the 98P7C3-encoding transcript.

### Example 43

### Purification of Naturally-occurring or Recombinant 98P7C3 Using 98P7C3 Specific Antibodies

Naturally occurring or recombinant 98P7C3 is substantially purified by immunoaffinity chromatography using antibodies specific for 98P7C3. An immunoaffinity column is constructed by covalently coupling anti-98P7C3 antibody to an activated chromatographic resin, such as CNBr-activated SEPHAROSE (Amersham Pharmacia Biotech). After the coupling, the resin is blocked and washed according to the manufacturer's instructions.

Media containing 98P7C3 are passed over the immunoaffinity column, and the column is washed under conditions that allow the preferential absorbance of 98P7C3 (e.g., high ionic strength buffers in the presence of detergent). The column is eluted under conditions that disrupt antibody/98P7C3 binding (e.g., a buffer of pH 2 to pH 3, or a high concentration of a chaotrope, such as urea or thiocyanate ion), and GCR.P is collected.

### Example 44

### Identification of Molecules Which Interact with 98P7C3

98P7C3, or biologically active fragments thereof, are labeled with 121 1 Bolton-Hunter reagent. (See, e.g., Bolton et al. (1973) Biochem. J. 133:529.) Candidate molecules previously arrayed in the wells of a multi-well plate are incubated with the labeled 98P7C3, washed, and any wells with labeled 98P7C3 complex are assayed. Data obtained using different concentrations of 98P7C3 are used to calculate values for the number, affinity, and association of 98P7C3 with the candidate molecules.

Throughout this application, various publications and applications are referenced. The disclosures of these publications and applications are hereby incorporated by reference herein in their entireties.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

TABLE I
Tissues that Express 98P7C3 When Malignant

| | |
|---|---|
| Prostate | Rectum |
| Kidney | Bone |
| Colon | Pancreas |
| Lung | Leukocytes |
| Breast | Ovary |
| Uterus | Bladder |
| Cervix | Metastasis |
| Stomach | |

TABLE II
AMINO ACID ABBREVIATIONS

| SINGLE LETTER | THREE LETTER | FULL NAME |
|---|---|---|
| F | Phe | phenylalanine |
| L | Leu | leucine |
| S | Ser | serine |
| Y | Tyr | tyrosine |
| C | Cys | cysteine |
| W | Trp | tryptophan |
| P | Pro | proline |
| H | His | histidine |
| Q | Gln | glutamine |
| R | Arg | arginine |
| I | Ile | isoleucine |
| M | Met | methionine |
| T | Thr | threonine |
| N | Asn | asparagine |
| K | Lys | lysine |
| V | Val | valine |
| A | Ala | alanine |
| D | Asp | aspartic acid |
| E | Glu | glutamic acid |
| G | Gly | glycine |

TABLE III
AMINO ACID SUBSTITUTION MATRIX
Adapted from the GCG Software 9.0 BLOSUM62 amino acid
substitution matrix (block substitution matrix).
The higher the value, the more likely a substitution
is found in related, natural proteins.

| A | C | D | E | F | G | H | I | K | L | M | N | P | Q | R | S | T | V | W | Y | |
|---|---|---|---|---|---|---|---|---|---|---|---|---|---|---|---|---|---|---|---|---|
| 4 | 0 | −2 | −1 | −2 | 0 | −2 | −1 | −1 | −1 | −1 | −2 | −1 | −1 | −1 | 1 | 0 | 0 | −3 | −2 | A |
| | 9 | −3 | −4 | −2 | −3 | −3 | −1 | −3 | −1 | −1 | −3 | −3 | −3 | −3 | −1 | −1 | −1 | −2 | −2 | C |
| | | 6 | 2 | −3 | −1 | −1 | −3 | −1 | −4 | −3 | 1 | −1 | 0 | −2 | 0 | −1 | −3 | −4 | −3 | D |
| | | | 5 | −3 | −2 | 0 | −3 | 1 | −3 | −2 | 0 | −1 | 2 | 0 | 0 | −1 | −2 | −3 | −2 | E |
| | | | | 6 | −3 | −1 | 0 | −3 | 0 | 0 | −3 | −4 | −3 | −3 | −2 | −2 | −1 | 1 | 3 | F |
| | | | | | 6 | −2 | −4 | −2 | −4 | −3 | 0 | −2 | −2 | −2 | 0 | −2 | −3 | −2 | −3 | G |
| | | | | | | 8 | −3 | −1 | −3 | −2 | 1 | −2 | 0 | 0 | −1 | −2 | −3 | −2 | 2 | H |
| | | | | | | | 4 | −3 | 2 | 1 | −3 | −3 | −3 | −3 | −2 | −1 | 3 | −3 | −1 | I |
| | | | | | | | | 5 | −2 | −1 | 0 | −1 | 1 | 2 | 0 | −1 | −2 | −3 | −2 | K |
| | | | | | | | | | 4 | 2 | −3 | −3 | −2 | −2 | −2 | −1 | 1 | −2 | −1 | L |
| | | | | | | | | | | 5 | −2 | −2 | 0 | −1 | −1 | −1 | 1 | −1 | −1 | M |
| | | | | | | | | | | | 6 | −2 | 0 | 0 | 1 | 0 | −3 | −4 | −2 | N |
| | | | | | | | | | | | | 7 | −1 | −2 | −1 | −1 | −2 | −4 | −3 | P |
| | | | | | | | | | | | | | 5 | 1 | 0 | −1 | −2 | −2 | −1 | Q |
| | | | | | | | | | | | | | | 5 | −1 | −1 | −3 | −3 | −2 | R |
| | | | | | | | | | | | | | | | 4 | 1 | −2 | −3 | −2 | S |
| | | | | | | | | | | | | | | | | 5 | 0 | −2 | −2 | T |
| | | | | | | | | | | | | | | | | | 4 | −3 | −1 | V |
| | | | | | | | | | | | | | | | | | | 11 | 2 | W |
| | | | | | | | | | | | | | | | | | | | 7 | Y |

TABLE IV (A)
HLA CLASS I SUPERMOTIFS

| SUPERMOTIF | POSITION 2 | C-TERMINUS |
|---|---|---|
| A2 | L, I, V, M, A, T, Q | L, I, V, M, A, T |
| A3 | A, V, I, L, M, S, T | R, K |
| B7 | P | A, L, I, M, V, F, W, Y |
| B44 | D, E | F, W, Y, L, I, M, V, A |
| A1 | T, S, L, I, V, M | F, W, Y |
| A24 | F, W, Y, L, V, I, M, T | F, I, Y, W, L, M |
| B27 | R, H, K | A, L, I, V, M, Y, F, W |
| B58 | A, S, T | F, W, Y, L, I, V |
| B62 | L, V, M, P, I, Q | F, W, Y, M, I, V |

TABLE IV (B)
HLA CLASS II SUPERMOTIF

| 1 | 6 | 9 |
|---|---|---|
| W, F, Y, V, I, L | A, V, I, L, P, C, S, T | A, V, I, L, C, S, T, M, Y |

TABLE V
HLA Peptide Scoring Results-98P7C3-A1, 9-mers

| | Start | Subsequence Residue | Score (Estimate of Half Time of Disassociation of a |
|---|---|---|---|
| Rank | Position | Listing | Molecule Containing This Subsequence) |
| 1 | 13 | QVEILEYNF | 18.000 |
| 2 | 42 | LSEEETQKW | 1.350 |
| 3 | 60 | RSEGLPSEC | 1.350 |
| 4 | 15 | EILEYNFNK | 1.000 |
| 5 | 16 | ILEYNFNKV | 0.900 |
| 6 | 44 | EEETQKWFK | 0.900 |
| 7 | 2 | SAETASGPT | 0.900 |
| 8 | 27 | HPDSTTLCL | 0.625 |
| 9 | 43 | SEEETQKWF | 0.450 |
| 10 | 9 | PTEDQVEIL | 0.225 |
| 11 | 41 | GLSEEETQK | 0.200 |
| 12 | 6 | ASGPTEDQV | 0.150 |
| 13 | 11 | EDQVEILEY | 0.125 |
| 14 | 30 | STTLCLIAA | 0.125 |
| 15 | 37 | AAEAGLSEE | 0.090 |
| 16 | 29 | DSTTLCLIA | 0.075 |
| 17 | 23 | KVDKHPDST | 0.050 |
| 18 | 65 | PSECRSVTD | 0.027 |
| 19 | 46 | ETQKWFKQR | 0.025 |
| 20 | 62 | EGLPSECRS | 0.025 |
| 21 | 32 | TLCLIAAEA | 0.020 |
| 22 | 34 | CLIAAEAGL | 0.020 |
| 23 | 18 | EYNFNKVDK | 0.020 |
| 24 | 10 | TEDQVEILE | 0.013 |
| 25 | 33 | LCLIAAEAG | 0.010 |
| 26 | 35 | LIAAEAGLS | 0.010 |
| 27 | 39 | EAGLSEEET | 0.010 |
| 28 | 5 | TASGPTEDQ | 0.010 |
| 29 | 63 | GLPSECRSV | 0.010 |
| 30 | 49 | KWFKQRLAK | 0.005 |
| 31 | 61 | SEGLPSECR | 0.005 |
| 32 | 31 | TTLCLIAAE | 0.005 |
| 33 | 36 | IAAEAGLSE | 0.005 |
| 34 | 8 | GPTEDQVEI | 0.005 |
| 35 | 25 | DKHPDSTTL | 0.005 |
| 36 | 19 | YNFNKVDKH | 0.003 |
| 37 | 40 | AGLSEEETQ | 0.003 |
| 38 | 4 | ETASGPTED | 0.003 |
| 39 | 64 | LPSECRSVT | 0.003 |
| 40 | 45 | EETQKWFKQ | 0.003 |
| 41 | 1 | MSAETASGP | 0.002 |
| 42 | 52 | KQRLAKWRR | 0.002 |
| 43 | 12 | DQVEILEYN | 0.002 |
| 44 | 22 | NKVDKHPDS | 0.001 |
| 45 | 55 | LAKWRRSEG | 0.001 |
| 46 | 51 | FKQRLAKWR | 0.001 |
| 47 | 26 | KHPDSTTLC | 0.001 |
| 48 | 53 | QRLAKWRRS | 0.001 |
| 49 | 14 | VEILEYNFN | 0.001 |
| 50 | 58 | WRRSEGLPS | 0.000 |

TABLE VI
HLA Peptide Scoring Results-98P7C3-A1, 10-mers

| | | | Score (Estimate of Half |
|---|---|---|---|
| | | | Time of Disassociation |
| | Start | Subsequence | of a Molecule Containing |
| Rank | Position | Residue Listing | This Subsequence) |
| 1 | 42 | LSEEEETQKWF | 13.500 |
| 2 | 60 | RSEGLPSECR | 13.500 |
| 3 | 10 | TEDQVEILEY | 6.250 |
| 4 | 43 | SEEETQKWFK | 0.900 |
| 5 | 13 | QVEILEYNFN | 0.900 |
| 6 | 44 | EEETQKWFKQ | 0.225 |
| 7 | 2 | SAETASGPTE | 0.180 |
| 8 | 27 | HPDSTTLCLI | 0.125 |
| 9 | 9 | PTEDQVEILE | 0.113 |
| 10 | 37 | AAEAGLSEEE | 0.090 |
| 11 | 16 | ILEYNFNKVD | 0.090 |
| 12 | 29 | DSTTLCLIAA | 0.075 |
| 13 | 14 | VEILEYNFNK | 0.050 |
| 14 | 23 | KVDKHPDSTT | 0.050 |
| 15 | 31 | TTLCLIAAEA | 0.050 |
| 16 | 40 | AGLSEEETQK | 0.050 |
| 17 | 12 | DQVEILEYNF | 0.030 |
| 18 | 4 | ETASGPTEDQ | 0.025 |
| 19 | 62 | EGLPSECRSV | 0.025 |
| 20 | 17 | LEYNFNKVDK | 0.020 |
| 21 | 33 | LCLIAAEAGL | 0.020 |
| 22 | 1 | MSAETASGPT | 0.015 |
| 23 | 6 | ASGPTEDQVE | 0.015 |
| 24 | 63 | GLPSECRSVT | 0.010 |
| 25 | 32 | TLCLIAAEAG | 0.010 |
| 26 | 15 | EILEYKFNKV | 0.010 |
| 27 | 34 | CLIAAEAGLS | 0.010 |
| 28 | 5 | TASGPTEDQV | 0.010 |
| 29 | 41 | GLSEEETQKW | 0.010 |
| 30 | 39 | EAGLSEEETQ | 0.010 |
| 31 | 48 | QKWFKQRLAK | 0.005 |
| 32 | 35 | LIAAEAGLSE | 0.005 |
| 33 | 25 | DKHPDSTTLC | 0.005 |
| 34 | 45 | EETQKWFKQR | 0.005 |
| 35 | 7 | SGPTEDQVEI | 0.005 |
| 36 | 30 | STTLCLIAAE | 0.005 |
| 37 | 26 | KHPDSTTLCL | 0.003 |
| 38 | 8 | GPTEDQVEIL | 0.003 |
| 39 | 46 | ETQKWFKQRL | 0.003 |
| 40 | 36 | IAAEAGLSEE | 0.001 |
| 41 | 54 | RLAKWRRSEG | 0.001 |
| 42 | 55 | LAKWRRSEGL | 0.001 |
| 43 | 50 | WFKQRLAKWR | 0.001 |
| 44 | 51 | FKQRLAKWRR | 0.001 |
| 45 | 64 | LPSECRSVTD | 0.001 |
| 46 | 38 | AEAGLSEEET | 0.001 |
| 47 | 18 | EYNFNKVDKH | 0.001 |
| 48 | 11 | EDQVEILEYN | 0.001 |
| 49 | 22 | NKVDKHPDST | 0.001 |
| 50 | 21 | FNKVDKHPDS | 0.001 |

TABLE VII
HLA Peptide Scoring Results-98P7C3-A2, 9-mers

| | | | Score (Estimate of Half |
|---|---|---|---|
| | | | Time of Disassociation |
| | Start | Subsequence | of a Molecule Containing |
| Rank | Position | Residue Listing | This Subsequence) |
| 1 | 34 | CLIAAEAGL | 21.362 |
| 2 | 16 | ILEYNFNKV | 14.378 |
| 3 | 63 | GLPSECRSV | 13.910 |
| 4 | 32 | TLCLIAAEA | 4.968 |
| 5 | 23 | KVDKHPDST | 0.447 |
| 6 | 47 | TQKWFKQRL | 0.288 |
| 7 | 8 | GPTEDQVEI | 0.279 |
| 8 | 6 | ASGPTEDQV | 0.222 |
| 9 | 30 | STTLCLIAA | 0.159 |
| 10 | 64 | LPSECRSVT | 0.139 |
| 11 | 56 | AKWRRSEGL | 0.121 |
| 12 | 41 | GLSEEETQK | 0.061 |
| 13 | 26 | KHPDSTTLC | 0.050 |
| 14 | 14 | VEILEYNFN | 0.020 |
| 15 | 12 | DQVEILEYN | 0.014 |
| 16 | 15 | EILEYNENK | 0.014 |
| 17 | 48 | QKWFKQRLA | 0.011 |
| 18 | 19 | YNFNKVDKH | 0.009 |
| 19 | 39 | EAGLSEEET | 0.004 |
| 20 | 52 | KQRLAKWRR | 0.003 |
| 21 | 54 | RLAKWRRSE | 0.003 |
| 22 | 17 | LEYNFNKVD | 0.002 |
| 23 | 35 | LIAAEAGLS | 0.002 |
| 24 | 25 | DKHPDSTTL | 0.002 |
| 25 | 43 | SEEETQKWF | 0.002 |
| 26 | 60 | RSEGLPSEC | 0.002 |
| 27 | 27 | HPDSTTLCL | 0.002 |
| 28 | 31 | TTLCLIAAE | 0.002 |
| 29 | 33 | LCLIAAEAG | 0.001 |
| 30 | 36 | IAAEAGLSE | 0.001 |
| 31 | 29 | DSTTLCLIA | 0.001 |
| 32 | 2 | SAETASGPT | 0.001 |
| 33 | 9 | PTEDQVEIL | 0.001 |
| 34 | 28 | PDSTTLCLI | 0.001 |
| 35 | 1 | MSAETASGP | 0.001 |
| 36 | 24 | VDKHPDSTT | 0.001 |
| 37 | 51 | FKQRLAKWR | 0.001 |
| 38 | 22 | NKVDKHPDS | 0.000 |
| 39 | 13 | QVEILEYNF | 0.000 |
| 40 | 3 | AETASGPTE | 0.000 |
| 41 | 42 | LSEEETQKW | 0.000 |
| 42 | 45 | EETQKWFKQ | 0.000 |
| 43 | 40 | AGLSEEETQ | 0.000 |
| 44 | 61 | SEGLPSECR | 0.000 |
| 45 | 38 | AEAGLSEEE | 0.000 |
| 46 | 62 | EGLPSECRS | 0.000 |
| 47 | 10 | TEDQVEILE | 0.000 |
| 48 | 5 | TASGPTEDQ | 0.000 |
| 49 | 55 | LAKWRRSEG | 0.000 |
| 50 | 44 | EEETQKWFK | 0.000 |

TABLE VIII
HLA Peptide Scoring Results-98P7C3-A2, 10-mers

| | Start | Subsequence Residue | Score (Estimate of Half Time of Disassociation of a |
|---|---|---|---|
| Rank | Position | Listing | Molecule Containing This Subsequence) |
| 1 | 15 | EILEYNFNKV | 20.673 |
| 2 | 63 | GLPSECRSVT | 3.055 |
| 3 | 23 | KVDKHPDSTT | 0.913 |
| 4 | 33 | LCLIAAEAGL | 0.877 |
| 5 | 5 | TASGPTEDQV | 0.473 |
| 6 | 41 | GLSEEETQKW | 0.306 |
| 7 | 8 | GPTEDQVEIL | 0.264 |
| 8 | 31 | TTLCLIAAEA | 0.255 |
| 9 | 26 | KHPDSTTLCL | 0.213 |
| 10 | 46 | ETQKWFKQRL | 0.085 |
| 11 | 54 | RLAKWRRSEG | 0.075 |
| 12 | 38 | AEAGLSEEET | 0.071 |
| 13 | 7 | SGPTEDQVEI | 0.068 |
| 14 | 1 | MSAETASGPT | 0.044 |
| 15 | 32 | TLCLIAAEAG | 0.037 |
| 16 | 62 | EGLPSECRSV | 0.025 |
| 17 | 22 | NKVDKHPDST | 0.021 |
| 18 | 34 | CLIAAEAGLS | 0.010 |
| 19 | 55 | LAKWRRSEGL | 0.009 |
| 20 | 29 | DSTTLCLIAA | 0.006 |
| 21 | 47 | TQKWFKQRLA | 0.004 |
| 22 | 35 | LIAAEAGLSE | 0.003 |
| 23 | 13 | QVEILEYNFN | 0.003 |
| 24 | 36 | IAAEAGLSEE | 0.003 |
| 25 | 43 | SEEETQKWFK | 0.003 |
| 26 | 14 | VEILEYNFNK | 0.003 |
| 27 | 59 | RRSEGLPSEC | 0.003 |
| 28 | 16 | ILEYNFNKVD | 0.003 |
| 29 | 12 | DQVEILEYNF | 0.002 |
| 30 | 27 | HPDSTTLCLI | 0.002 |
| 31 | 24 | VDKHPDSTTL | 0.002 |
| 32 | 19 | YNFNKVDKHP | 0.002 |
| 33 | 17 | LEYNFNKVDK | 0.002 |
| 34 | 61 | SEGLPSECRS | 0.001 |
| 35 | 10 | TEDQVEILEY | 0.001 |
| 36 | 42 | LSEEETQKWF | 0.001 |
| 37 | 51 | FKQRLAKWRR | 0.001 |
| 38 | 52 | KQRLAKWRRS | 0.001 |
| 39 | 64 | LPSECRSVTD | 0.001 |
| 40 | 25 | DKHPDSTTLC | 0.001 |
| 41 | 30 | STTLCLIAAE | 0.000 |
| 42 | 40 | AGLSEEETQK | 0.000 |
| 43 | 49 | KWFKQRLAKW | 0.000 |
| 44 | 3 | AETASGPTED | 0.000 |
| 45 | 48 | QKWFKQRLAK | 0.000 |
| 46 | 21 | FNKVDKHPDS | 0.000 |
| 47 | 28 | PDSTTLCLIA | 0.000 |
| 48 | 11 | EDQVEILEYN | 0.000 |
| 49 | 45 | EETQKWFKQR | 0.000 |
| 50 | 6 | ASGPTEDQVE | 0.000 |

TABLE IX
HLA Peptide Scoring Results-98P7C3-A3, 9-mers

| | Start | Subsequence Residue | Score (Estimate of Half Time of Disassociation of a |
|---|---|---|---|
| Rank | Position | Listing | Molecule Containing This Subsequence) |
| 1 | 41 | GLSEEETQK | 90.000 |
| 2 | 15 | EILEYNFNK | 12.150 |
| 3 | 52 | KQRLAKWRR | 1.080 |
| 4 | 34 | CLIAAEAGL | 0.900 |
| 5 | 49 | KWFKQRLAK | 0.600 |
| 6 | 13 | QVEILEYNF | 0.600 |
| 7 | 16 | ILEYNFNKV | 0.600 |
| 8 | 32 | TLCLIAAEA | 0.300 |
| 9 | 46 | ETQKWFKQR | 0.270 |
| 10 | 63 | GLPSECRSV | 0.060 |
| 11 | 47 | TQKWFKQRL | 0.054 |
| 12 | 44 | EEETQKWFK | 0.054 |
| 13 | 23 | KVDKHPDST | 0.030 |
| 14 | 30 | STTLCLIAA | 0.030 |
| 15 | 8 | GPTEDQVEI | 0.027 |
| 16 | 61 | SEGLPSECR | 0.018 |
| 17 | 27 | HPDSTTLCL | 0.012 |
| 18 | 9 | PTEDQVEIL | 0.009 |
| 19 | 19 | YNFNKVDKH | 0.006 |
| 20 | 54 | RLAKWRRSE | 0.006 |
| 21 | 18 | EYNFNKVDK | 0.006 |
| 22 | 43 | SEEETQKWF | 0.005 |
| 23 | 11 | EDQVEILEY | 0.004 |
| 24 | 56 | AKWRRSEGL | 0.003 |
| 25 | 31 | TTLCLIAAE | 0.002 |
| 26 | 51 | FKQRLAKWR | 0.002 |
| 27 | 64 | LPSECRSVT | 0.002 |
| 28 | 6 | ASGPTEDQV | 0.002 |
| 29 | 60 | RSEGLPSEC | 0.002 |
| 30 | 42 | LSEEETQKW | 0.002 |
| 31 | 35 | LIAAEAGLS | 0.001 |
| 32 | 12 | DQVEILEYN | 0.001 |
| 33 | 36 | IAAEAGLSE | 0.001 |
| 34 | 26 | KHPDSTTLC | 0.001 |
| 35 | 29 | DSTTLCLIA | 0.001 |
| 36 | 17 | LEYNFNKVD | 0.000 |
| 37 | 5 | TASGPTEDQ | 0.000 |
| 38 | 50 | WFKQRLAKW | 0.000 |
| 39 | 37 | AAEAGLSEE | 0.000 |
| 40 | 33 | LCLIAAEAG | 0.000 |
| 41 | 4 | ETASGPTED | 0.000 |
| 42 | 2 | SAETASGPT | 0.000 |
| 43 | 39 | EAGLSEEET | 0.000 |
| 44 | 25 | DKHPDSTTL | 0.000 |
| 45 | 55 | LAKWRRSEG | 0.000 |
| 46 | 10 | TEDQVEILE | 0.000 |
| 47 | 1 | MSAETASGP | 0.000 |
| 48 | 24 | VDKHPDSTT | 0.000 |
| 49 | 48 | QKWFKQRLA | 0.000 |
| 50 | 28 | PDSTTLCLI | 0.000 |

TABLE X
HLA Peptide Scoring Results-98P7C3-A3, 10-mers

| | Start | Subsequence Residue | Score (Estimate of Half Time of Disassociation of a |
|---|---|---|---|
| Rank | Position | Listing | Molecule Containing This Subsequence) |
| 1 | 41 | GLSEEETQKW | 0.900 |
| 2 | 14 | VEILEYNFNK | 0.810 |
| 3 | 63 | GLPSECRSVT | 0.300 |
| 4 | 17 | LEYNFNKVDK | 0.300 |
| 5 | 43 | SEEETQKWFK | 0.270 |
| 6 | 48 | QKWFKQRLAK | 0.200 |
| 7 | 12 | DQVEILEYNF | 0.121 |
| 8 | 8 | GPTEDQVEIL | 0.081 |
| 9 | 60 | RSEGLPSECR | 0.045 |
| 10 | 23 | KVDKHPDSTT | 0.045 |
| 11 | 15 | EILEYNFNKV | 0.041 |
| 12 | 10 | TEDQVEILEY | 0.036 |
| 13 | 40 | AGLSEEETQK | 0.030 |
| 14 | 16 | ILEYNFNKVD | 0.030 |
| 15 | 46 | ETQKWFKQRL | 0.027 |
| 16 | 31 | TTLCLIAAEA | 0.022 |
| 17 | 54 | RLAKWRRSEG | 0.020 |
| 18 | 32 | TLCLIAAEAG | 0.020 |
| 19 | 34 | CLIAAEAGLS | 0.018 |
| 20 | 45 | EETQKWFKQR | 0.016 |
| 21 | 51 | FKQRLAKWRR | 0.012 |
| 22 | 27 | HPDSTTLCLI | 0.009 |
| 23 | 33 | LCLIAAEAGL | 0.009 |
| 24 | 42 | LSEEETQKWF | 0.007 |
| 25 | 55 | LAKWRRSEGL | 0.006 |
| 26 | 49 | KWFKQRLAKW | 0.005 |
| 27 | 35 | LIAAEAGLSE | 0.004 |
| 28 | 26 | KHPDSTTLCL | 0.004 |
| 29 | 5 | TASGPTEDQV | 0.003 |
| 30 | 50 | WFKQRLAKWR | 0.002 |
| 31 | 13 | QVEILEYNFN | 0.002 |
| 32 | 30 | STTLCLIAAE | 0.002 |
| 33 | 29 | DSTTLCLIAA | 0.001 |
| 34 | 47 | TQKWFKQRLA | 0.001 |
| 35 | 24 | VDKHPDSTTL | 0.001 |
| 36 | 7 | SGPTEDQVEI | 0.001 |
| 37 | 36 | IAAEAGLSEE | 0.000 |
| 38 | 4 | ETASGPTEDQ | 0.000 |
| 39 | 38 | AEAGLSEEET | 0.000 |
| 40 | 52 | KQRLAKWRRS | 0.000 |
| 41 | 64 | LPSECRSVTD | 0.000 |
| 42 | 9 | PTEDQVEILE | 0.000 |
| 43 | 59 | RRSEGLPSEC | 0.000 |
| 44 | 57 | KWRRSEGLPS | 0.000 |
| 45 | 1 | MSAETASGPT | 0.000 |
| 46 | 37 | AAEAGLSEEE | 0.000 |
| 47 | 2 | SAETASGPTE | 0.000 |
| 48 | 18 | EYNFNKVDKH | 0.000 |
| 49 | 19 | YNFNKVDKHP | 0.000 |
| 50 | 6 | ASGPTEDQVE | 0.000 |

TABLE XI
HLA Peptide Scoring Results - 98P7C3 - A11, 9-mers

| | | | Score (Estimate of Half Time of |
|---|---|---|---|
| | Start | Subsequence Residue | Disassociation of a Molecule |
| Rank | Position | Listing | Containing This Subsequence) |
| 1 | 41 | GLSEEETQK | 1.200 |
| 2 | 52 | KQRLAKWRR | 0.720 |
| 3 | 15 | EILEYNFNK | 0.540 |
| 4 | 49 | KWFKQRLAK | 0.240 |
| 5 | 18 | EYNFNKVDK | 0.120 |
| 6 | 13 | QVEILEYNF | 0.040 |
| 7 | 44 | EEETQKWFK | 0.036 |
| 8 | 46 | ETQKWFKQR | 0.030 |
| 9 | 30 | STTLCLIAA | 0.020 |
| 10 | 61 | SEGLPSECR | 0.012 |
| 11 | 34 | CLIAAEAGL | 0.006 |
| 12 | 47 | TQKWFKQRL | 0.006 |
| 13 | 8 | GPTEDQVEI | 0.006 |
| 14 | 23 | KVDKHPDST | 0.006 |
| 15 | 27 | HPDSTTLCL | 0.004 |
| 16 | 16 | ILEYNFNKV | 0.004 |
| 17 | 32 | TLCLIAAEA | 0.004 |
| 18 | 51 | FKQRLAKWR | 0.002 |
| 19 | 50 | WFKQRLAKW | 0.002 |
| 20 | 31 | TTLCLIAAE | 0.002 |
| 21 | 63 | GLPSECRSV | 0.001 |
| 22 | 9 | PTEDQVEIL | 0.001 |
| 23 | 19 | YNFNKVDKH | 0.001 |
| 24 | 56 | AKWRRSEGL | 0.000 |
| 25 | 35 | LIAAEAGLS | 0.000 |
| 26 | 36 | IAAEAGLSE | 0.000 |
| 27 | 4 | ETASGPTED | 0.000 |
| 28 | 33 | LCLIAAEAG | 0.000 |
| 29 | 43 | SEEETQKWF | 0.000 |
| 30 | 12 | DQVEILEYN | 0.000 |
| 31 | 5 | TASGPTEDQ | 0.000 |
| 32 | 6 | ASGPTEDQV | 0.000 |
| 33 | 2 | SAETASGPT | 0.000 |
| 34 | 37 | AAEAGLSEE | 0.000 |
| 35 | 42 | LSEEETQKW | 0.000 |
| 36 | 55 | LAKWRRSEG | 0.000 |
| 37 | 64 | LPSECRSVT | 0.000 |
| 38 | 54 | RLAKWRRSE | 0.000 |
| 39 | 10 | TEDQVEILE | 0.000 |
| 40 | 29 | DSTTLCLIA | 0.000 |
| 41 | 11 | EDQVEILEY | 0.000 |
| 42 | 20 | NFNKVDKHP | 0.000 |
| 43 | 14 | VEILEYNFN | 0.000 |
| 44 | 60 | RSEGLPSEC | 0.000 |
| 45 | 26 | KHPDSTTLC | 0.000 |
| 46 | 57 | KWRRSEGLP | 0.000 |
| 47 | 59 | RRSEGLPSE | 0.000 |
| 48 | 17 | LEYNFNKVD | 0.000 |
| 49 | 39 | EAGLSEEET | 0.000 |
| 50 | 25 | DKHPDSTTL | 0.000 |

TABLE XII
HLA Peptide Scoring Results - 98P7C3 - A11, 10-mers

| | | | Score (Estimate of Half Time of |
|---|---|---|---|
| | Start | Subsequence Residue | Disassociation of a Molecule |
| Rank | Position | Listing | Containing This Subsequence) |
| 1 | 14 | VEILEYNFNK | 0.270 |
| 2 | 17 | LEYNFNKVDK | 0.120 |
| 3 | 43 | SEEETQKWFK | 0.120 |
| 4 | 48 | QKWFKQRLAK | 0.080 |
| 5 | 40 | AGLSEEETQK | 0.030 |
| 6 | 50 | WFKQRLAKWR | 0.020 |
| 7 | 31 | TTLCLIAAEA | 0.015 |
| 8 | 60 | RSEGLPSECR | 0.012 |
| 9 | 41 | GLSEEETQKW | 0.012 |
| 10 | 51 | FKQRLAKWRR | 0.008 |
| 11 | 8 | GPTEDQVEIL | 0.006 |
| 12 | 23 | KVDKHPDSTT | 0.006 |
| 13 | 12 | DQVEILEYNF | 0.005 |
| 14 | 33 | LCLIAAEAGL | 0.003 |
| 15 | 46 | ETQKWFKQRL | 0.003 |
| 16 | 27 | HPDSTTLCLI | 0.002 |
| 17 | 13 | QVEILEYNFN | 0.002 |
| 18 | 55 | LAKWRRSEGL | 0.002 |
| 19 | 5 | TASGPTEDQV | 0.002 |
| 20 | 45 | EETQKWFKQR | 0.002 |
| 21 | 15 | EILEYNFNKV | 0.002 |
| 22 | 26 | KHPDSTTLCL | 0.001 |
| 23 | 63 | GLPSECRSVT | 0.001 |
| 24 | 54 | RLAKWRRSEG | 0.001 |
| 25 | 49 | KWFKQRLAKW | 0.001 |
| 26 | 10 | TEDQVEILEY | 0.001 |
| 27 | 18 | EYNFNKVDKH | 0.001 |
| 28 | 30 | STTLCLIAAE | 0.001 |
| 29 | 35 | LIAAEAGLSE | 0.001 |
| 30 | 47 | TQKWFKQRLA | 0.001 |
| 31 | 34 | CLIAAEAGLS | 0.001 |
| 32 | 32 | TLCLIAAEAG | 0.000 |
| 33 | 4 | ETASGPTEDQ | 0.000 |
| 34 | 16 | ILEYNFNKVD | 0.000 |
| 35 | 9 | PTEDQVEILE | 0.000 |
| 36 | 20 | NFNKVDKHPD | 0.000 |
| 37 | 24 | VDKHPDSTTL | 0.000 |
| 38 | 36 | IAAEAGLSEE | 0.000 |
| 39 | 64 | LPSECRSVTD | 0.000 |
| 40 | 2 | SAETASGPTE | 0.000 |
| 41 | 37 | AAEAGLSEEE | 0.000 |
| 42 | 7 | SGPTEDQVEI | 0.000 |
| 43 | 52 | KQRLAKWRRS | 0.000 |
| 44 | 57 | KWRRSEGLPS | 0.000 |
| 45 | 29 | DSTTLCLIAA | 0.000 |
| 46 | 42 | LSEEETQKWF | 0.000 |
| 47 | 59 | RRSEGLPSEC | 0.000 |
| 48 | 61 | SEGLPSECRS | 0.000 |
| 49 | 38 | AEAGLSEEET | 0.000 |
| 50 | 3 | AETASGPTED | 0.000 |

TABLE XIII
HLA Peptide Scoring Results - 98P7C3 - A24, 9-mers

| | | | Score (Estimate of Half Time of |
|---|---|---|---|
| | Start | Subsequence Residue | Disassociation of a Molecule |
| Rank | Position | Listing | Containing This Subsequence) |
| 1 | 34 | CLIAAEAGL | 6.000 |
| 2 | 47 | TQKWFKQRL | 4.800 |
| 3 | 27 | HPDSTTLCL | 4.000 |
| 4 | 13 | QVEILEYNF | 3.600 |
| 5 | 8 | GPTEDQVEI | 1.320 |
| 6 | 18 | EYNFNKVDK | 0.750 |
| 7 | 9 | PTEDQVEIL | 0.720 |
| 8 | 50 | WFKQRLAKW | 0.550 |
| 9 | 25 | DKHPDSTTL | 0.480 |
| 10 | 56 | AKWRRSEGL | 0.400 |
| 11 | 60 | RSEGLPSEC | 0.396 |
| 12 | 43 | SEEETQKWF | 0.360 |
| 13 | 12 | DQVEILEYN | 0.252 |
| 14 | 16 | ILEYNFNKV | 0.238 |
| 15 | 42 | LSEEETQKW | 0.238 |
| 16 | 23 | KVDKHPDST | 0.200 |
| 17 | 62 | EGLPSECRS | 0.180 |
| 18 | 32 | TLCLIAAEA | 0.154 |
| 19 | 63 | GLPSECRSV | 0.150 |
| 20 | 2 | SAETASGPT | 0.150 |
| 21 | 29 | DSTTLCLIA | 0.120 |
| 22 | 64 | LPSECRSVT | 0.120 |
| 23 | 6 | ASGPTEDQV | 0.120 |
| 24 | 39 | EAGLSEEET | 0.110 |
| 25 | 20 | NFNKVDKHP | 0.105 |
| 26 | 30 | STTLCLIAA | 0.100 |
| 27 | 35 | LIAAEAGLS | 0.100 |
| 28 | 26 | KHPDSTTLC | 0.036 |
| 29 | 54 | RLAKWRRSE | 0.020 |
| 30 | 52 | KQRLAKWRR | 0.020 |
| 31 | 57 | KWRRSEGLP | 0.020 |
| 32 | 49 | KWFKQRLAK | 0.020 |
| 33 | 14 | VEILEYNFN | 0.018 |
| 34 | 22 | NKVDKHPDS | 0.018 |
| 35 | 15 | EILEYNFNK | 0.018 |
| 36 | 31 | TTLCLIAAE | 0.018 |
| 37 | 7 | SGPTEDQVE | 0.018 |
| 38 | 46 | ETQKWFKQR | 0.018 |
| 39 | 11 | EDQVEILEY | 0.017 |
| 40 | 37 | AAEAGLSEE | 0.017 |
| 41 | 33 | LCLIAAEAG | 0.015 |
| 42 | 40 | AGLSEEETQ | 0.015 |
| 43 | 53 | QRLAKWRRS | 0.015 |
| 44 | 41 | GLSEEETQK | 0.012 |
| 45 | 1 | MSAETASGP | 0.012 |
| 46 | 36 | IAAEAGLSE | 0.012 |
| 47 | 19 | YNFNKVDKH | 0.011 |
| 48 | 4 | ETASGPTED | 0.011 |
| 49 | 55 | LAKWRRSEG | 0.011 |
| 50 | 58 | WRRSEGLPS | 0.010 |

TABLE XIV
HLA Peptide Scoring Results - 98P7C3 - A24, 10-mers

| | | | Score (Estimate of Half Time of |
|---|---|---|---|
| | Start | Subsequence Residue | Disassociation of a Molecule |
| Rank | Position | Listing | Containing This Subsequence) |
| 1 | 46 | ETQKWFKQRL | 7.200 |
| 2 | 33 | LCLIAAEAGL | 6.000 |
| 3 | 8 | GPTEDQVEIL | 4.800 |
| 4 | 12 | DQVEILEYNF | 4.320 |
| 5 | 55 | LAKWRRSEGL | 4.000 |
| 6 | 42 | LSEEETQKWF | 3.600 |
| 7 | 7 | SGPTEDQVEI | 1.650 |
| 8 | 26 | KHPDSTTLCL | 1.440 |
| 9 | 27 | HPDSTTLCLI | 1.000 |
| 10 | 18 | EYNFNKVDKH | 0.825 |
| 11 | 24 | VDKHPDSTTL | 0.400 |
| 12 | 15 | EILEYNFNKV | 0.285 |
| 13 | 31 | TTLCLIAAEA | 0.231 |
| 14 | 49 | KWFKQRLAKW | 0.220 |
| 15 | 52 | KQRLAKWRRS | 0.200 |
| 16 | 57 | KWRRSEGLPS | 0.200 |
| 17 | 23 | KVDKHPDSTT | 0.200 |
| 18 | 62 | EGLPSECRSV | 0.180 |
| 19 | 13 | QVEILEYNFN | 0.180 |
| 20 | 41 | GLSEEETQKW | 0.158 |
| 21 | 63 | GLPSECRSVT | 0.150 |
| 22 | 34 | CLIAAEAGLS | 0.150 |
| 23 | 1 | MSAETASGPT | 0.120 |
| 24 | 29 | DSTTLCLIAA | 0.100 |
| 25 | 21 | FNKVDKHPDS | 0.100 |
| 26 | 5 | TASGPTEDQV | 0.100 |
| 27 | 47 | TQKWFKQRLA | 0.100 |
| 28 | 20 | NFNKVDKHPD | 0.075 |
| 29 | 50 | WFKQRLAKWR | 0.060 |
| 30 | 59 | RRSEGLPSEC | 0.032 |
| 31 | 60 | RSEGLPSECR | 0.030 |
| 32 | 54 | RLAKWRRSEG | 0.022 |
| 33 | 11 | EDQVEILEYN | 0.021 |
| 34 | 37 | AAEAGLSEEE | 0.020 |
| 35 | 22 | NKVDKHPDST | 0.018 |
| 36 | 16 | ILEYNFNKVD | 0.015 |
| 37 | 2 | SAETASGPTE | 0.015 |
| 38 | 40 | AGLSEEETQK | 0.015 |
| 39 | 6 | ASGPTEDQVE | 0.014 |
| 40 | 19 | YNFNKVDKHP | 0.014 |
| 41 | 36 | IAAEAGLSEE | 0.013 |
| 42 | 64 | LPSECRSVTD | 0.012 |
| 43 | 30 | STTLCLIAAE | 0.012 |
| 44 | 25 | DKHPDSTTLC | 0.012 |
| 45 | 10 | TEDQVEILEY | 0.011 |
| 46 | 38 | AEAGLSEEET | 0.011 |
| 47 | 35 | LIAAEAGLSE | 0.010 |
| 48 | 32 | TLCLIAAEAG | 0.010 |
| 49 | 39 | EAGLSEEETQ | 0.010 |
| 50 | 4 | ETASGPTEDQ | 0.010 |

TABLE XV
HLA Peptide Scoring Results - 98P7C3 - B7, 9-mers

| | | | Score (Estimate of Half Time of |
|---|---|---|---|
| | Start | Subsequence Residue | Disassociation of a Molecule |
| Rank | Position | Listing | Containing This Subsequence) |
| 1 | 27 | HPDSTTLCL | 24.000 |
| 2 | 8 | GPTEDQVEI | 8.000 |
| 3 | 47 | TQKWFKQRL | 4.000 |
| 4 | 34 | CLIAAEAGL | 4.000 |
| 5 | 64 | LPSECRSVT | 3.000 |
| 6 | 56 | AKWRRSEGL | 1.200 |
| 7 | 6 | ASGPTEDQV | 0.600 |
| 8 | 25 | DKHPDSTTL | 0.400 |
| 9 | 39 | EAGLSEEET | 0.300 |
| 10 | 63 | GLPSECRSV | 0.200 |
| 11 | 23 | KVDKHPDST | 0.150 |
| 12 | 9 | PTEDQVEIL | 0.120 |
| 13 | 30 | STTLCLIAA | 0.100 |
| 14 | 32 | TLCLIAAEA | 0.100 |
| 15 | 29 | DSTTLCLIA | 0.100 |
| 16 | 52 | KQRLAKWRR | 0.100 |
| 17 | 2 | SAETASGPT | 0.090 |
| 18 | 16 | ILEYNFNKV | 0.060 |
| 19 | 55 | LAKWRRSEG | 0.045 |
| 20 | 5 | TASGPTEDQ | 0.045 |
| 21 | 13 | QVEILEYNF | 0.030 |
| 22 | 40 | AGLSEEETQ | 0.030 |
| 23 | 36 | IAAEAGLSE | 0.030 |
| 24 | 60 | RSEGLPSEC | 0.030 |
| 25 | 37 | AAEAGLSEE | 0.027 |
| 26 | 58 | WRRSEGLPS | 0.020 |
| 27 | 12 | DQVEILEYN | 0.020 |
| 28 | 62 | EGLPSECRS | 0.020 |
| 29 | 35 | LIAAEAGLS | 0.020 |
| 30 | 24 | VDKHPDSTT | 0.015 |
| 31 | 54 | RLAKWRRSE | 0.015 |
| 32 | 46 | ETQKWFKQR | 0.010 |
| 33 | 7 | SGPTEDQVE | 0.010 |
| 34 | 31 | TTLCLIAAE | 0.010 |
| 35 | 41 | GLSEEETQK | 0.010 |
| 36 | 4 | ETASGPTED | 0.010 |
| 37 | 15 | EILEYNFNK | 0.010 |
| 38 | 57 | KWRRSEGLP | 0.010 |
| 39 | 1 | MSAETASGP | 0.010 |
| 40 | 48 | QKWFKQRLA | 0.010 |
| 41 | 33 | LCLIAAEAG | 0.010 |
| 42 | 21 | FNKVDKHPD | 0.010 |
| 43 | 26 | KHPDSTTLC | 0.010 |
| 44 | 19 | YNFNKVDKH | 0.010 |
| 45 | 42 | LSEEETQKW | 0.006 |
| 46 | 28 | PDSTTLCLI | 0.004 |
| 47 | 38 | AEAGLSEEE | 0.003 |
| 48 | 3 | AETASGPTE | 0.003 |
| 49 | 14 | VEILEYNFN | 0.002 |
| 50 | 11 | EDQVEILEY | 0.002 |

TABLE XVI
HLA Peptide Scoring Results-98P7C3-B7, 10-mers

| | | | Score (Estimate of Half |
|---|---|---|---|
| | | | Time of Disassociation |
| | Start | Subsequence | of a Molecule Containing |
| Rank | Position | Residue Listing | This Subsequence) |
| 1 | 8 | GPTEDQVEIL | 80.000 |
| 2 | 55 | LAKWRRSEGL | 12.000 |
| 3 | 46 | ETQKWPKQRL | 4.000 |
| 4 | 33 | LCLIAAEAGL | 4.000 |
| 5 | 27 | HPDSTTLCLI | 2.400 |
| 6 | 5 | TASGPTEDQV | 0.600 |
| 7 | 7 | SGPTEDQVEI | 0.400 |
| 8 | 26 | KHPDSTTLCL | 0.400 |
| 9 | 24 | VDKHPDSTTL | 0.400 |
| 10 | 23 | KVDKHPDSTT | 0.225 |
| 11 | 15 | EILEYNFNKV | 0.200 |
| 12 | 64 | LPSECRSVTD | 0.200 |
| 13 | 52 | KQRLAKWRRS | 0.200 |
| 14 | 62 | EGLPSECRSV | 0.200 |
| 15 | 63 | GLPSECRSVT | 0.150 |
| 16 | 29 | DSTTLCLIAA | 0.100 |
| 17 | 31 | ITLCLIAAEA | 0.100 |
| 18 | 47 | TQKWFKQRLA | 0.100 |
| 19 | 1 | MSAETASGPT | 0.100 |
| 20 | 38 | AEAGLSEEET | 0.030 |
| 21 | 13 | QVEILEYNFN | 0.030 |
| 22 | 39 | EAGLSEEETQ | 0.030 |
| 23 | 40 | AGLSEEETQK | 0.030 |
| 24 | 36 | IAAEAGLSEE | 0.030 |
| 25 | 6 | ASGPTEDQVE | 0.030 |
| 26 | 37 | AAEAGLSEEE | 0.027 |
| 27 | 57 | KWRRSEGLPS | 0.020 |
| 28 | 12 | DQVEILEYNF | 0.020 |
| 29 | 21 | FNKVDKHPDS | 0.020 |
| 30 | 34 | CLIAAEAGLS | 0.020 |
| 31 | 41 | GLSEEETQKW | 0.020 |
| 32 | 4 | ETASGPTEDQ | 0.015 |
| 33 | 54 | RLAKWRRSEG | 0.015 |
| 34 | 35 | LIAAEAGLSE | 0.010 |
| 35 | 59 | RRSEGLPSEC | 0.010 |
| 36 | 30 | STTLCLIAAE | 0.010 |
| 37 | 32 | TLCLIAAEAG | 0.010 |
| 38 | 22 | NKVDKHPDST | 0.010 |
| 39 | 25 | DKHPDSTTLC | 0.010 |
| 40 | 58 | WRRSEGLPSE | 0.010 |
| 41 | 19 | YNFNKVDKHP | 0.010 |
| 42 | 2 | SAETASGPTE | 0.009 |
| 43 | 42 | LSEEETQKWF | 0.006 |
| 44 | 60 | RSEGLPSECR | 0.004 |
| 45 | 3 | AETASGPTED | 0.003 |
| 46 | 56 | AKWRRSEGLP | 0.003 |
| 47 | 16 | ILEYNFNKVD | 0.003 |
| 48 | 49 | KWFKQRLAKW | 0.002 |
| 49 | 61 | SEGLPSECRS | 0.002 |
| 50 | 11 | EDQVEILEYN | 0.002 |

TABLE XVII
HLA Peptide Scoring Results - 98P7C3 - B35, 9-mers

| | | | Score (Estimate of Half Time of |
|---|---|---|---|
| | Start | Subsequence Residue | Disassociation of a Molecule |
| Rank | Position | Listing | Containing This Subsequence) |
| 1 | 8 | GPTEDQVEI | 24.000 |
| 2 | 27 | HPDSTTLCL | 6.000 |
| 3 | 64 | LPSECRSVT | 4.000 |
| 4 | 47 | TQKWFKQRL | 3.000 |
| 5 | 42 | LSEEETQKW | 2.250 |
| 6 | 34 | CLIAAEAGL | 1.000 |
| 7 | 6 | ASGPTEDQV | 1.000 |
| 8 | 29 | DSTTLCLIA | 0.500 |
| 9 | 39 | EAGLSEEET | 0.300 |
| 10 | 60 | RSEGLPSEC | 0.300 |
| 11 | 11 | EDQVEILEY | 0.300 |
| 12 | 13 | QVEILEYNF | 0.300 |
| 13 | 63 | GLPSECRSV | 0.300 |
| 14 | 12 | DQVEILEYN | 0.200 |
| 15 | 35 | LIAAEAGLS | 0.150 |
| 16 | 25 | DKHPDSTTL | 0.150 |
| 17 | 50 | WFKQRLAKW | 0.150 |
| 18 | 56 | AKWRRSEGL | 0.100 |
| 19 | 32 | TLCLIAAEA | 0.100 |
| 20 | 1 | MSAETASGP | 0.100 |
| 21 | 30 | STTLCLIAA | 0.100 |
| 22 | 62 | EGLPSECRS | 0.100 |
| 23 | 55 | LAKWRRSEG | 0.090 |
| 24 | 2 | SAETASGPT | 0.090 |
| 25 | 52 | KQRLAKWRR | 0.060 |
| 26 | 36 | IAAEAGLSE | 0.060 |
| 27 | 9 | PTEDQVEIL | 0.060 |
| 28 | 16 | ILEYNFNKV | 0.060 |
| 29 | 23 | KVDKHPDST | 0.060 |
| 30 | 43 | SEEETQKWF | 0.060 |
| 31 | 58 | WRRSEGLPS | 0.045 |
| 32 | 21 | FNKVDKHPD | 0.045 |
| 33 | 26 | KHPDSTTLC | 0.040 |
| 34 | 41 | GLSEEETQK | 0.030 |
| 35 | 5 | TASGPTEDQ | 0.030 |
| 36 | 24 | VDKHPDSTT | 0.030 |
| 37 | 22 | NKVDKHPDS | 0.020 |
| 38 | 54 | RLAKWRRSE | 0.020 |
| 39 | 15 | EILEYNFNK | 0.020 |
| 40 | 14 | VEILEYNFN | 0.015 |
| 41 | 40 | AGLSEEETQ | 0.015 |
| 42 | 7 | SGPTEDQVE | 0.015 |
| 43 | 46 | ETQKWFKQR | 0.010 |
| 44 | 19 | YNFNKVDKH | 0.010 |
| 45 | 4 | ETASGPTED | 0.010 |
| 46 | 33 | LCLIAAEAG | 0.010 |
| 47 | 53 | QRLAKWRRS | 0.010 |
| 48 | 31 | TTLCLIAAE | 0.010 |
| 49 | 48 | QKWFKQRLA | 0.010 |
| 50 | 37 | AAEAGLSEE | 0.009 |

TABLE XVIII
HLA Peptide Scoring Results - 98P7C3 - B35, 10-mers

| | | | Score (Estimate of Half Time of |
|---|---|---|---|
| | Start | Subsequence Residue | Disassociation of a Molecule |
| Rank | Position | Listing | Containing This Subsequence) |
| 1 | 8 | GPTEDQVEIL | 40.000 |
| 2 | 55 | LAKWRRSEGL | 9.000 |
| 3 | 42 | LSEEETQKWF | 3.000 |
| 4 | 27 | HPDSTTLCLI | 2.400 |
| 5 | 12 | DQVEILEYNF | 2.000 |
| 6 | 41 | GLSEEETQKW | 1.500 |
| 7 | 33 | LCLIAAEAGL | 1.000 |
| 8 | 46 | ETQKWFKQRL | 1.000 |
| 9 | 1 | MSAETASGPT | 1.000 |
| 10 | 52 | KQRLAKWRRS | 0.600 |
| 11 | 7 | SGPTEDQVEI | 0.600 |
| 12 | 5 | TASGPTEDQV | 0.600 |
| 13 | 29 | DSTTLCLIAA | 0.500 |
| 14 | 24 | VDKHPDSTTL | 0.450 |
| 15 | 15 | EILEYNFNKV | 0.400 |
| 16 | 26 | KHPDSTTLCL | 0.400 |
| 17 | 64 | LPSECRSVTD | 0.400 |
| 18 | 62 | EGLPSECRSV | 0.300 |
| 19 | 21 | FNKVDKHPDS | 0.300 |
| 20 | 47 | TQKWFKQRLA | 0.300 |
| 21 | 34 | CLIAAEAGLS | 0.150 |
| 22 | 31 | TTLCLIAAEA | 0.100 |
| 23 | 63 | GLPSECRSVT | 0.100 |
| 24 | 49 | KWFKQRLAKW | 0.100 |
| 25 | 57 | KWRRSEGLPS | 0.090 |
| 26 | 10 | TEDQVEILEY | 0.090 |
| 27 | 6 | ASGPTEDQVE | 0.075 |
| 28 | 36 | IAAEAGLSEE | 0.060 |
| 29 | 23 | KVDKHPDSTT | 0.060 |
| 30 | 39 | EAGLSEEETQ | 0.045 |
| 31 | 13 | QVEILEYNFN | 0.045 |
| 32 | 59 | RRSEGLPSEC | 0.040 |
| 33 | 60 | RSEGLPSECR | 0.030 |
| 34 | 54 | RLAKWRRSEG | 0.020 |
| 35 | 22 | NKVDKHPDST | 0.020 |
| 36 | 40 | AGLSEEETQK | 0.015 |
| 37 | 11 | EDQVEILEYN | 0.010 |
| 38 | 4 | ETASGPTEDQ | 0.010 |
| 39 | 32 | TLCLIAAEAG | 0.010 |
| 40 | 38 | AEAGLSEEET | 0.010 |
| 41 | 30 | STTLCLIAAE | 0.010 |
| 42 | 25 | DKHPDSTTLC | 0.010 |
| 43 | 35 | LIAAEAGLSE | 0.010 |
| 44 | 19 | YNFNKVDKHP | 0.010 |
| 45 | 61 | SEGLPSECRS | 0.010 |
| 46 | 37 | AAEAGLSEEE | 0.009 |
| 47 | 2 | SAETASGPTE | 0.009 |
| 48 | 50 | WFKQRLAKWR | 0.003 |
| 49 | 58 | WRRSEGLPSE | 0.003 |
| 50 | 16 | ILEYNFNKVD | 0.003 |

### Table XIX: Motif-bearing Subsequences of the 98P7C3 Protein

Protein Motifs

Protein kinase C phosphorylation site

47-49 TQK

Casein kinase II phosphorylation site

Number of matches: 2

1 43-46 SEEE

2 70-73 SVTD

TABLE XX
Frequently Occurring Motifs

| | av. % | | |
|---|---|---|---|
| Name | identity | Description | Potential Function |
| zf-C2H2 | 34% | Zinc finger, | Nucleic acid-binding protein |
| | | C2H2 type | functions as transcription |
| | | | factor, nuclear location |
| | | | probable |
| cytochrome | 68% | Cytochrome b | membrane bound oxidase, |
| b N | | (N-terminal)/ | generate superoxide |
| | | b6/petB | |
| ig | 19% | Immunoglobulin | domains are one hundred |
| | | domain | amino acids long and |
| | | | include a conserved |
| | | | intradomain disulfide |
| | | | bond. |
| WD40 | 18% | WD domain, | tandem repeats of about |
| | | G-beta repeat | 40 residues, each containing |
| | | | a Trp-Asp motif. Function in |
| | | | signal transduction and |
| | | | protein interaction |
| PDZ | 23% | PDZ domain | may function in targeting |
| | | | signaling molecules to sub- |
| | | | memebranous sites |
| LRR | 28% | Leucine Rich | short sequence motifs |
| | | Repeat | involved in protein-protein |
| pkinase | 23% | Protein kinase | interactions conserved |
| | | domain | catalytic core common |
| | | | to both serine/theonine |
| | | | and tyrosine protein kinases |
| | | | containing kinases containing |
| | | | an ATP binding site and a |
| | | | catalytic site |
| PH | 16% | PH domain | pleckstrin homology involved |
| | | | in intercellular signaling or |
| | | | as constituents of the |
| | | | cytoskeleton |
| EGF | 34% | EGF-like | 30-40 amino-acid long found |
| | | domain | in the extracellular domain |
| | | | of membrane-bound proteins |
| | | | or in secreted proteins |
| rvt | 49% | Reverse | |
| | | transcriptase | |
| | | (RNA-dependent | |
| | | DNA polymerase) | |
| ank | 25% | Ank repeat | Cytoplasmic protein, |
| | | | associates integral membrane |
| | | | proteins to the cytoskeleton |
| oxidored ql | 32% | NADH- | membrane associated. In- |
| | | Ubiquinone/ | volved in proton translocation |
| | | plastoquinone | across the membrane |
| | | (complex I), | |
| | | various chains | |
| efhand | 24% | EF hand | calcium-binding domain, |
| | | | consists of a 12 residue loop |
| | | | flanked on both sides by a 12 |
| | | | residue alpha-helical domain |
| rvp | 79% | Retroviral | Aspartyl or acid proteases, |
| | | aspartyl | centered on a catalytic |
| | | protease | aspartyl residue |
| Collagen | 42% | Collagen | extracellular structural |
| | | triple helix | proteins involved in formation |
| | | repeat | of connective tissue. The |
| | | (20 copies) | sequence consists of the |
| | | | G-X-Y and the polypeptide |
| | | | chains forms a triple helix. |
| fn3 | 20% | Fibronectin | Located in the extracellular |
| | | type III | ligand-binding region of |
| | | domain | receptors and is about 200 |
| | | | amino acid residues long with |
| | | | two pairs of cysteines |
| | | | involved in disulfide bonds |
| 7tm 1 | 19% | 7 transmembrane | seven hydrophobic |
| | | receptor | transmembrane regions, with |
| | | (rhodopsin | the N-terminus located |
| | | family) | extracellularly while the |
| | | | C-terminus is cytoplasmic. |
| | | | Signal through G proteins |

1. An isolated 98P7C3-related protein.

2. The 98P7C3-related protein of claim 1, wherein the 98P7C3-related protein has at least 6 contiguous amino acids of an amino acid sequence shown in SEQ ID NO: 2.

3. The 98P7C3-related protein of claim 1, wherein 98P7C3-related protein has at least 7, 8, 9, 10, 11, 12, 13, 14, 15, 16, 17, 18, 19, 20, 21, 22, 23, 24, 25, or more than 25 contiguous amino acids of an amino acid sequence shown in SEQ ID NO: 2.

4. The 98P7C3-related protein of claim 1, wherein the 98P7C3-related protein is at least 30, 35, 40, 45, 50, 55, 60, 65, 70 or more than 70 contiguous amino acids of an amino acid sequence shown in SEQ ID NO: 2.

5. The 98P7C3-related protein of claim 1, wherein the 98P7C3-related protein includes an amino acid sequence selected from the group consisting of amino acid residues 43-46, 47-49, 70-73, 10-60, of SEQ ID NO: 2.

6. An 98P7C3-related protein of claim 1 that comprises an HLA class I A1, A2, A3, A24, B7, B27, B58, B62 supermotif, or an HLA class II DR supermotif set forth in Table IV (B) or an Alexander pan DR binding epitope supermotif or an HLA DR3 motif.

7. An 98P7C3-related protein of claim 1 that comprises at least one conservative substitution.

8. An 98P7C3-related protein of claim 1 that comprises an epitope that induces a specific antibody response.

9. The 98P7C3-related protein of claim 1, wherein the 98P7C3-related protein has the amino acid sequence shown in SEQ ID NO: 2.

10. The 98P7C3-related protein that is at least 90% homologous to an amino acid sequence of claim 1.

11. The 98P7C3-related protein that is at least 90% identical to an amino acid sequence of claim 1.

12. An isolated 98P7C3-related protein of claim 1 that has an amino acid sequence which is exactly that of an amino acid sequence encoded by a polynucleotide selected from the group consisting of:
(a) a polynucleotide consisting of the sequence as shown in SEQ ID NO: 1, wherein T can also be U;
(b) a polynucleotide consisting of the sequence as shown in SEQ ID NO: 1, from nucleotide residue number 141 through nucleotide residue number 362, wherein T can also be U;
(c) a polynucleotide that encodes a 98P7C3-related protein whose sequence is encoded by the cDNAs contained in the plasmid designated p98P7C3-EBB10 deposited with American Type Culture Collection as Accession No. PTA-1261;
(d) a polynucleotide that encodes an 98P7C3-related protein that is at least 90% homologous to the entire amino acid sequence shown in SEQ ID NO: 2;
(e) a polynucleotide that encodes an 98P7C3-related protein that is at least 90% identical to the entire amino acid sequence shown in SEQ ID NO: 2;
(f) a polynucleotide that is fully complementary to a polynucleotide of any one of (a)-(e); and,
(g) a polynucleotide that selectively hybridizes under stringent conditions to a polynucleotide of (a)-(e).

13. A pharmaceutical composition comprising a 98P7C3 polynucleotide and a pharmaceutically acceptable carrier, wherein the polynucleotide is selected from the group consisting of:
(a) a polynucleotide consisting of the sequence as shown in SEQ ID NO: 1, wherein T can also be U;
(b) a polynucleotide consisting of the sequence as shown in SEQ ID NO: 1, from nucleotide residue number 141 through nucleotide residue number 362, wherein T can also be U;
(c) a polynucleotide that encodes a 98P7C3-related protein whose sequence is encoded by the cDNAs contained in the plasmid designated p98P7C3 -EBB 10 deposited with American Type Culture Collection as Accession No. PTA-1261;
(d) a polynucleotide that encodes an 98P7C3-related protein that is at least 90% homologous to the entire amino acid sequence shown in SEQ ED NO: 2;
(e) a polynucleotide that encodes an 98P7C3-related protein that is at least 90% identical to the entire amino acid sequence shown in SEQ ID NO: 2;
(f) a polynucleotide that is fully complementary to a polynucleotide of any one of (a)-(e); and,
(g) a polynucleotide that selectively hybridizes under stringent conditions to a polynucleotide of (a)-(e).

14. A pharmaceutical composition of claim 13, wherein the polynucleotide encodes a 98P7C3-related protein, and wherein the polynucleotide consists of the sequence as shown in FIG. 2 (SEQ ID NO: 1), from nucleotide residue number 141 through nucleotide residue number 362, wherein T can also be U.

15. A pharmaceutical composition of claim 13, wherein the polynucleotide encodes a 98P7C3-related protein, wherein the polypeptide includes an amino acid sequence selected from the group consisting of amino acid residues 43-46, 47-49, 70-73, 10-60, of SEQ ID NO: 2.

16. A pharmaceutical composition of claim 13, wherein the polynucleotide encodes an 98P7C3-related protein, wherein the protein comprises an HLA class I A1, A2, A3, A24, B7, B27, B58, B62 supermotif, or an HLA class II DR supermotif set forth in Table IV (B) or an Alexander pan DR binding epitope supermotif or an HLA DR3 motif.

17. A pharmaceutical composition comprising a recombinant expression vector that contains a polynucleotide of claim 13, and a pharmaceutically acceptable carrier.

18. A pharmaceutical composition comprising a host cell that contains an expression vector of claim 17, and a pharmaceutically acceptable carrier.

19. A process for producing a 98P7C3-related protein comprising culturing a host cell of claim 18 under conditions sufficient for the production of the polypeptide and recovering the 98P7C3-related protein so produced.

20. A 98P7C3-related protein produced by the process of claim 19.

21. An antibody or fragment thereof that specifically binds to a 98P7C3-related protein.

22. The antibody or fragment thereof of claim 24, that specifically binds to a portion of the 98P7C3-related protein, wherein the portion is selected from the group consisting of amino acid residues 43-46, 47-49, 70-73, 10-60 of SEQ ID NO: 2.

23. The antibody or fragment thereof of claim 21, which is monoclonal.

24. A recombinant protein comprising the antigen-binding region of a monoclonal antibody of claim 23.

25. The antibody or fragment thereof of claim 21, which is labeled with a detectable marker.

26. The recombinant protein of claim 24, which is labeled with a detectable marker.

27. The antibody fragment of claim 21, which is an Fab, F(ab′)2, Fv or Sfv fragment.

28. The antibody of claim 21, which is a human antibody.

29. The recombinant protein of claim 24, which comprises murine antigen binding region residues and human constant region residues.

30. A non-human transgenic animal that produces an antibody of claim 21.

31. A hybridoma that produces an antibody of claim 23.

32. A single chain monoclonal antibody that comprises the variable domains of the heavy and light chains of a monoclonal antibody of claim 23.

33. A vector comprising a polynucleotide that encodes a single chain monoclonal antibody of claim 32 that immunospecifically binds to a 98P7C3-related protein.

34. An assay for detecting the presence of a 98P7C3-related protein or polynucleotide in a biological sample comprising steps of:
contacting the sample with an antibody or another polynucleotide, respectively, that specifically binds to the 98P7C3-related protein or polynucleotide, respectively; and,
detecting the binding of 98P7C3-related protein or polynucleotide, respectively, in the sample thereto.

35. An assay of claim 34 for detecting the presence of an 98P7C3-related protein or polynucleotide comprising the steps of:
obtaining a sample,
evaluating said sample in the presence of an 98P7C3-related protein or polynucleotide, whereby said evaluating step produces a result that indicates the presence or amount of 98P7C3-related protein or polynucleotide, respectively.

36. An assay of claim 35 for detecting the presence of an 98P7C3 polynucleotide in a biological sample, comprising:
(a) contacting the sample with a polynucleotide probe that specifically hybridizes to a polynucleotide encoding an 98P7C3-related protein having an amino acid sequence shown in FIG. 2; and
(b) detecting the presence of a hybridization complex formed by the hybridization of the probe with 98P7C3 polynucleotide in the sample, wherein the presence of the hybridization complex indicates the presence of 98P7C3 polynucleotide within the sample.

37. An assay for detecting the presence of 98P7C3 mRNA in a biological sample comprising:
(a) producing cDNA from the sample by reverse transcription using at least one primer;
(b) amplifying the cDNA so produced using 98P7C3 polynucleotides as sense and antisense primers to amplify 98P7C3 cDNAs therein, wherein the 98P7C3 polynucleotides used as the sense and antisense probes are capable of amplifying the 98P7C3 cDNA contained within the plasmid as deposited with American Type Culture Collection as Accession No. PTA-1261;
(c) detecting the presence of the amplified 98P7C3 cDNA,

38. A method for monitoring 98P7C3 gene products comprising:
determining the status of 98P7C3 gene products expressed by cells in a tissue sample from an individual;
comparing the status so determined to the status of 98P7C3 gene products in a corresponding normal sample; and
identifying the presence of aberrant 98P7C3 gene products in the sample relative to the normal sample.

39. A method of monitoring the presence of cancer in an individual comprising:
performing the method of claim 38 whereby the presence of elevated 98P7C3 mRNA or protein expression in the test sample relative to the normal tissue sample provides an indication of the presence or status of a cancer.

40. The method of claim 39, wherein the cancer occurs in a tissue set forth in Table I.

41. A pharmaceutical composition comprising a substance that modulates the status of a cell that expresses 98P7C3.

42. A pharmaceutical composition of claim 41 that comprises an 98P7C3-related protein and a physiologically acceptable carrier.

43. A pharmaceutical composition of claim 41 that comprises an antibody or fragment thereof that specifically binds to a 98P7C3-related protein and a physiologically acceptable carrier.

44. A pharmaceutical composition of claim 41 that comprises a polynucleotide that encodes a single chain monoclonal antibody that immunospecifically binds to an 98P7C3-related protein and a physiologically acceptable carrier.

45. A pharmaceutical composition of claim 41 that comprises a polynucleotide comprising a 98P7C3-related protein coding sequence and a physiologically acceptable carrier.

46. A pharmaceutical composition of claim 41 that comprises an antisense polynucleotide complementary to a polynucleotide having a 98P7C3 coding sequence and a physiologically acceptable carrier.

47. A pharmaceutical composition of claim 41 that comprises a ribozyme capable of cleaving a polynucleotide having 98P7C3 coding sequence and a physiologically acceptable carrier.

48. A method of treating a patient with a cancer that expresses 98P7C3, the method comprising steps of:
administering to said patient a vector that comprises the composition of claim 44, such that the vector delivers the single chain monoclonal antibody coding sequence to the cancer cells and the encoded single chain antibody is expressed intracellularly therein.

49. A method of inhibiting in a patient the development of a cancer that expresses 98P7C3, the method comprising:
administering to the patient an effective amount of the composition of claim 41.

50. A method of generating an immune response directed to 98P7C3 in a mammal, the method comprising:
exposing the mammal's immune system to an immunogenic portion of an 98P7C3-related protein of claim 1 or a nucleotide sequence that encodes said protein, whereby an immune response is generated to 98P7C3.

51. A method of delivering a cytotoxic agent to a cell that expresses 98P7C3, said method comprising:
conjugating the cytotoxic agent to an antibody or fragment thereof of claim 21 that specifically binds to a 98P7C3 epitope; and,
exposing the cell to the antibody-agent conjugate.

52. A method of inducing an immune response to an 98P7C3 protein, said method comprising:
providing a 98P7C3-related protein of claim 1 that comprises at least one T cell or at least one B cell epitope;
contacting the epitope with an immune system T cell or B cell respectively, whereby the immune system T cell or B cell is induced.

53. The method of claim 52, wherein the immune system cell is a B cell, whereby the induced B cell generates antibodies that specifically bind to the 98P7C3 -related protein.

54. The method of claim 53, wherein the immune system cell is a T cell that is a cytotoxic T cell (CTL), whereby the activated CTL kills an autologous cell that expresses the 98P7C3 protein.

55. The method of claim 52, wherein the immune system cell is a T cell that is a helper T cell (HTL), whereby the activated HTL secretes cytokines that facilitate the cytotoxic activity of a CTL or the antibody producing activity of a B cell.
